(19) United States
(12) Patent Application Publication
(10) Pub. No.: US 2025/0188128 A1
(43) Pub. Date: Jun. 12, 2025

(54) ADENO-ASSOCIATED VIRUS COMPOSITIONS HAVING INCREASED HEART ENRICHMENT

(71) Applicant: CAPSIDA, INC., Thousand Oaks, CA (US)

(72) Inventors: Brandon G. Wheeler, Thousand Oaks, CA (US); Xiaojing Shi, Thousand Oaks, CA (US); Nicholas S. Goeden, Thousand Oaks, CA (US); Nicholas C. Flytzanis, Thousand Oaks, CA (US); Troy E. Sandberg, Thousand Oaks, CA (US)

(21) Appl. No.: 18/843,978

(22) PCT Filed: Mar. 24, 2023
(86) PCT No.: PCT/US2023/016254

§ 371 Date: Sep. 4, 2024

Related U.S. Application Data
(60) Provisional application No. 63/323,406, filed on Mar. 24, 2022.

(51) Int. Cl. C07K 14/005; A61K 38/00; C12N 15/86

(52) U.S. Cl. CPC C07K 14/005; C12N 15/86; A61K 38/00; C12N 2750/14322; C12N 2750/14343

(57) ABSTRACT

Described herein are compositions and kits comprising recombinant adeno-associated viruses (rAAVs) with increased M transduction enrichment in the heart. The rAAV compositions described herein encapsidate a transgene, such as a therapeutic nucleic acid. Gene therapy using the rAAVs is described. Also described are methods of treating cardiovascular-related diseases and conditions.

### BACKGROUND

Recombinant adeno-associated viruses (rAAVs) are widely used as vectors for gene delivery in therapeutic applications because of their ability to transduce both dividing and non-dividing cells, their long-term persistence as episomal DNA in infected cells, and their low immunogenicity. These characteristics make them appealing for applications in therapeutic applications, such as gene therapy. However, there is a need to significantly improve the performance of existing AAV serotypes to selectively and efficiently express in distinct cell-types, upon systemic delivery to a subject. This need is especially acute when the AAV must be expressed in the heart and vascular tissue.

### SUMMARY OF THE INVENTION

Disclosed herein are rAAVs with engineered transduction into the capsid structure through iterative rounds of selection in non-human primates (NHPs), yielding variants having increased transduction when measured in the heart.

The present invention provides rAAVs with widespread transduction to the heart and vasculature.

The present invention provides, in an aspect, a peptide insertion sequence comprising or consisting of an amino-acid sequence set forth in any one of Tables 1-2, FIG. 1 and and/or Formulas I-VIII.

Another aspect of the invention is a modified capsid protein wherein the AAV capsid protein, with a peptide insertion comprising or consisting of an amino-acid sequence set forth in any one of Tables 1-2, FIG. 1 and and/or Formulas I-VIII, is characterized by increased heart transduction in a subject.

Another aspect of the invention is a modified capsid protein wherein the AAV capsid protein, with a peptide insertion comprising or consisting of an amino-acid sequence set forth in any one of Tables 1-2, FIG. 1 and and/or Formulas I-VIII, is characterized by increased enrichment in heart tissue in a subject.

The present disclosure moreover includes pharmaceutical compositions comprising rAAVs with a peptide insertion comprising or consisting of an amino-acid sequence set forth in any one of Tables 1-2, FIG. 1 and and/or Formulas I-VII, and a pharmaceutically acceptable excipient.

Aspects disclosed herein provide methods of treating a disease or condition in a subject comprising administering a therapeutically effective amount of a pharmaceutical formulation comprising the AAV capsid protein or the AAV capsid of the present disclosure. In some embodiments, the disease or the condition is a disease or a condition of the heart, and cardiovascular tissue of the subject. Relatedly, the invention includes use of the rAAVs in the manufacture of a medicament for treating or preventing the disease or medical condition.

Other aspects of the invention will be apparent from the detailed description and claims that follow.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows AAV capsid protein insertion and substitution amino acid sequences encoding the amino acid sequences which were found in the non-human primate heart tissue after two rounds of evaluation of engineered AAV libraries.

### DETAILED DESCRIPTION OF THE DISCLOSURE

In one aspect the disclosure provides rAAVs with high expression levels in the heart.

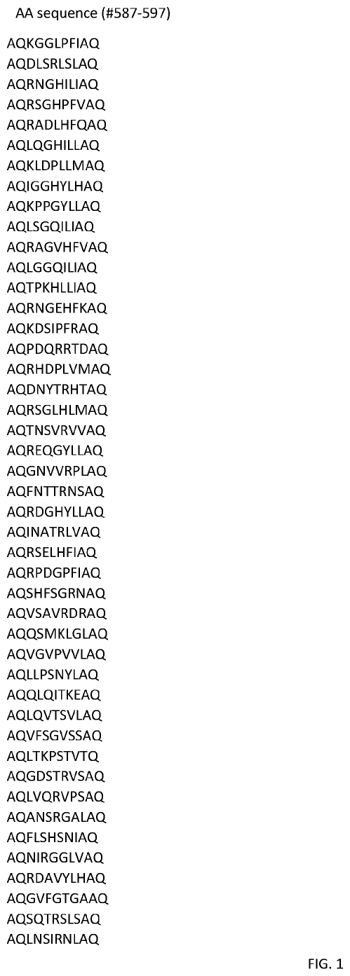

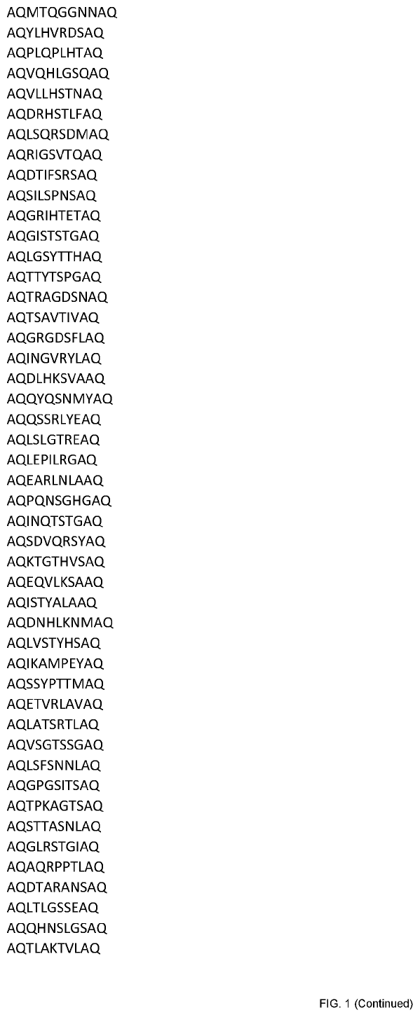

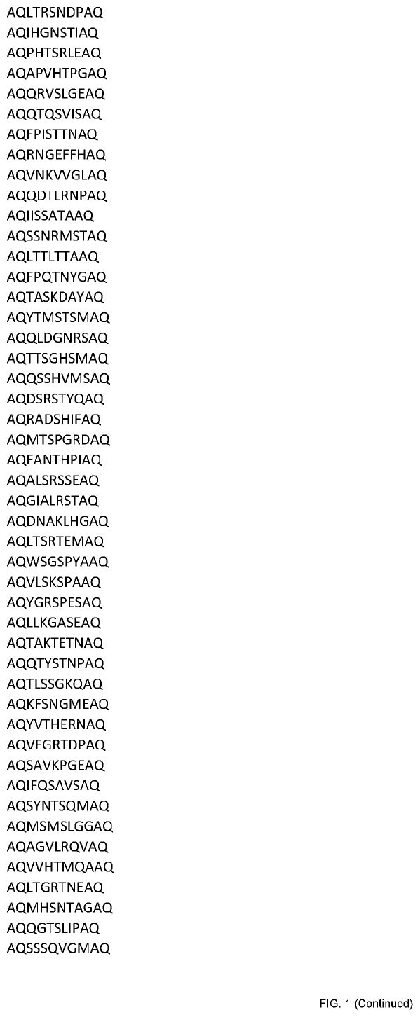

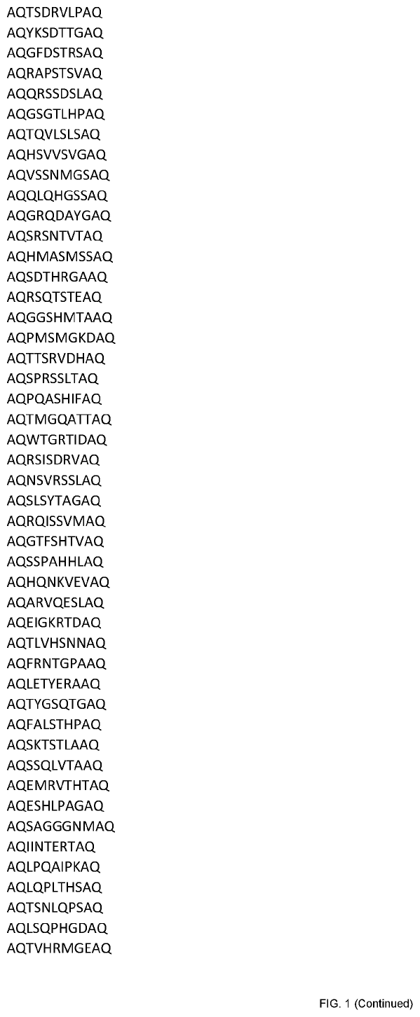

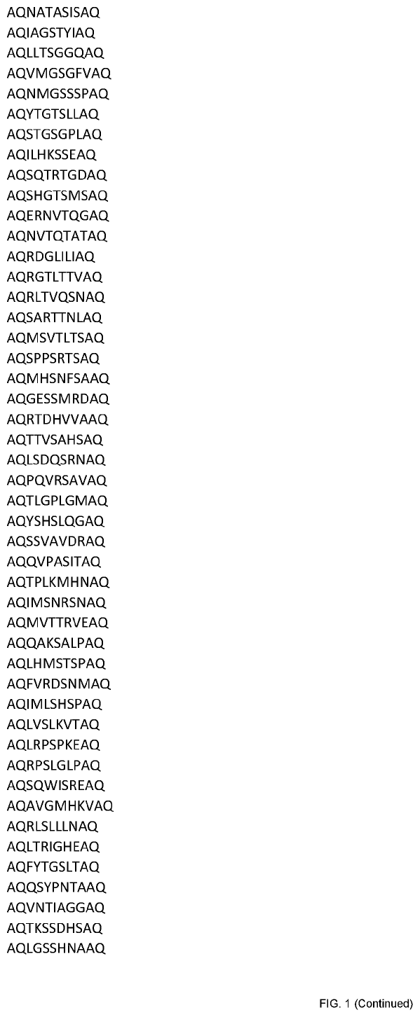

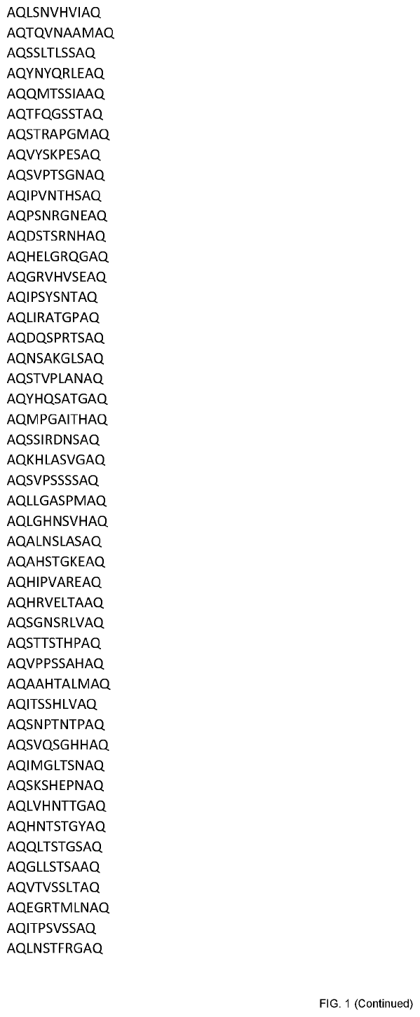

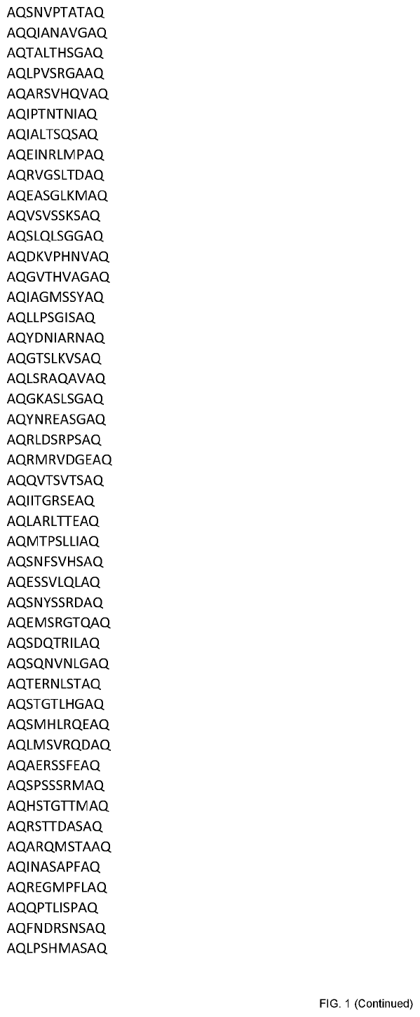

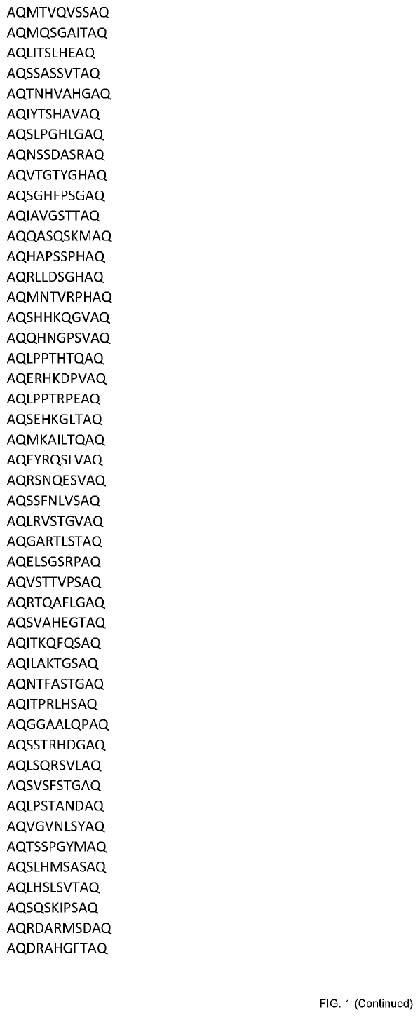

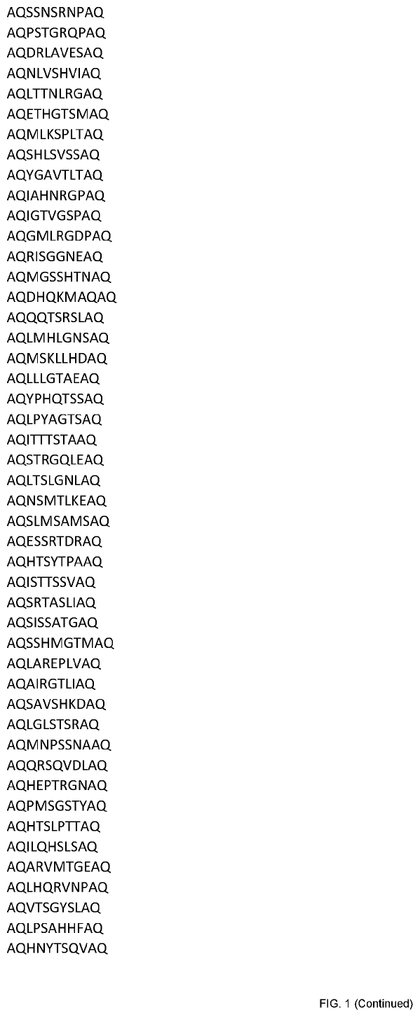

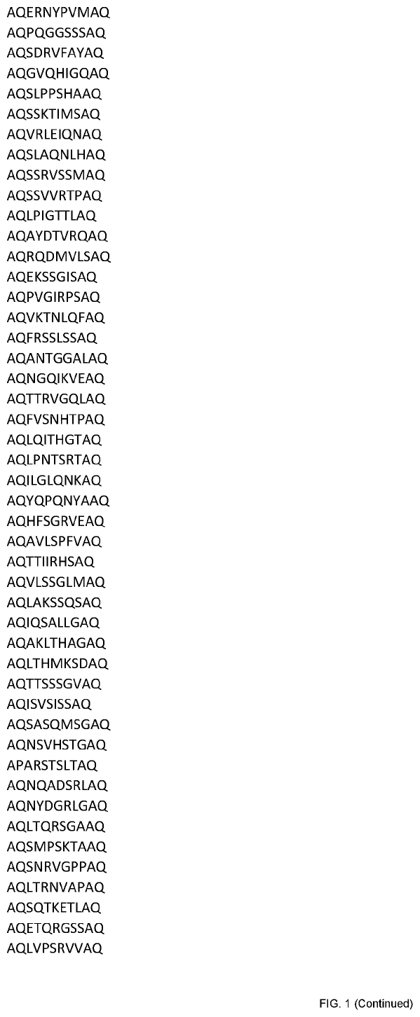

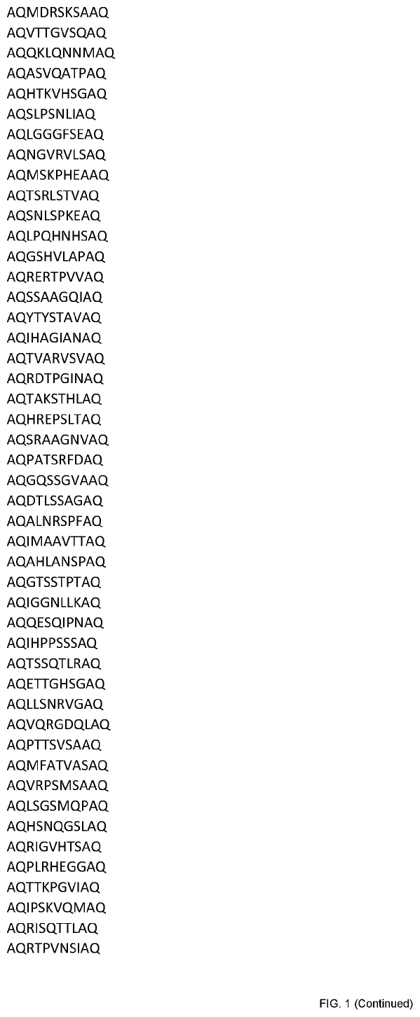

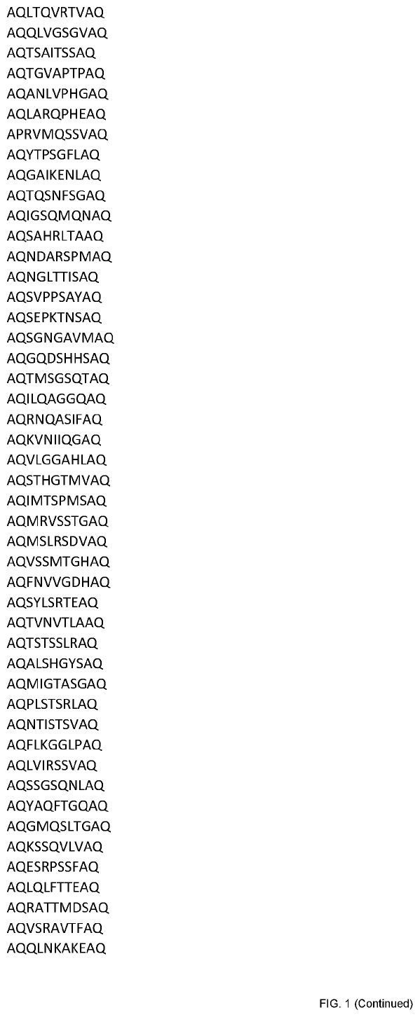

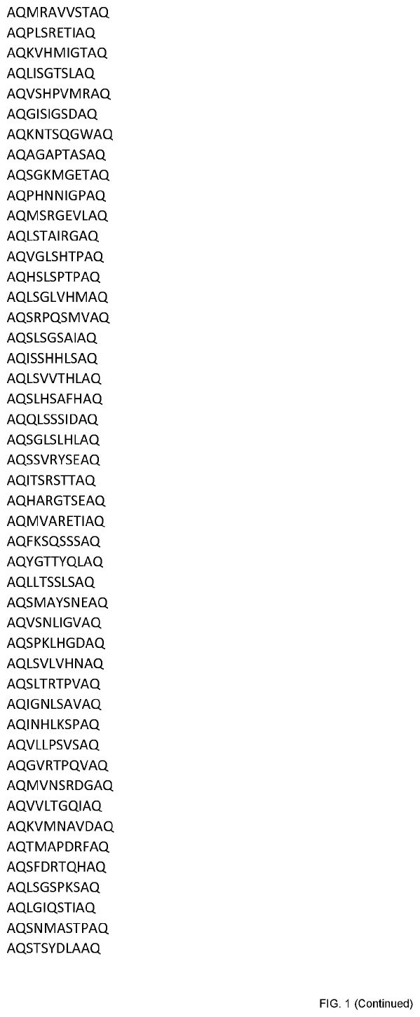

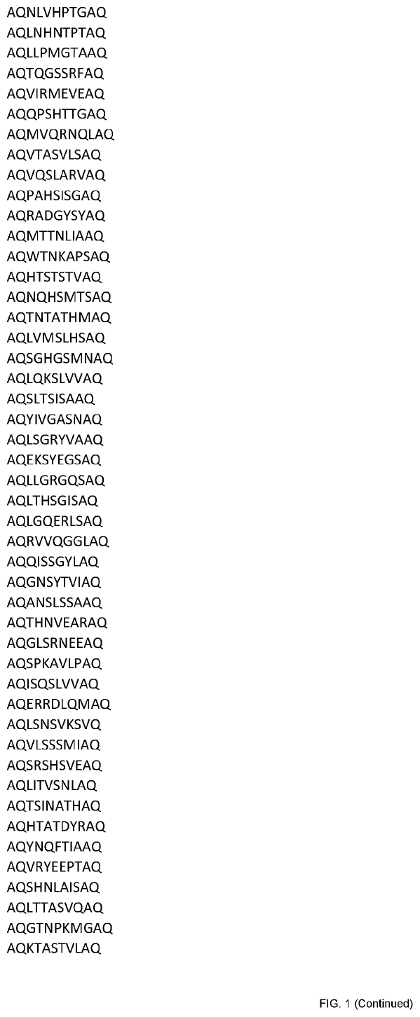

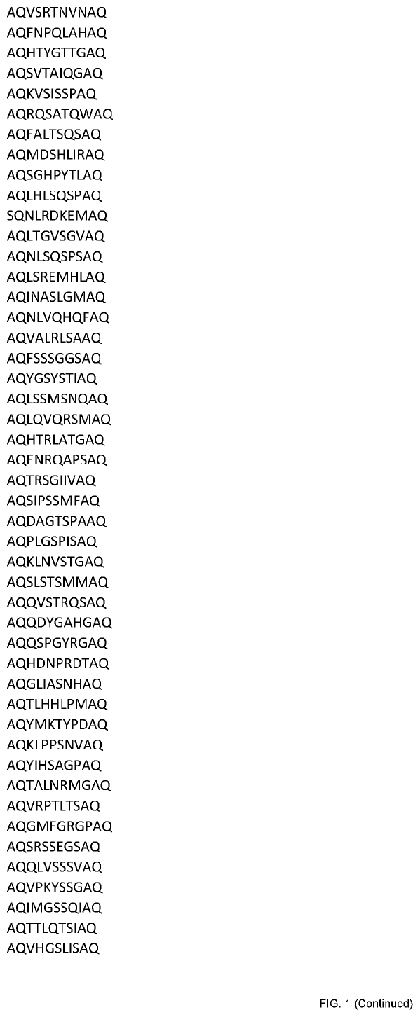

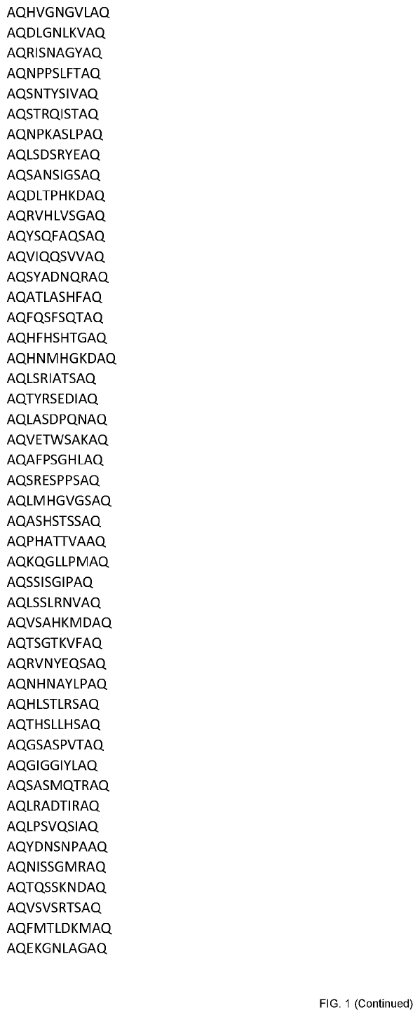

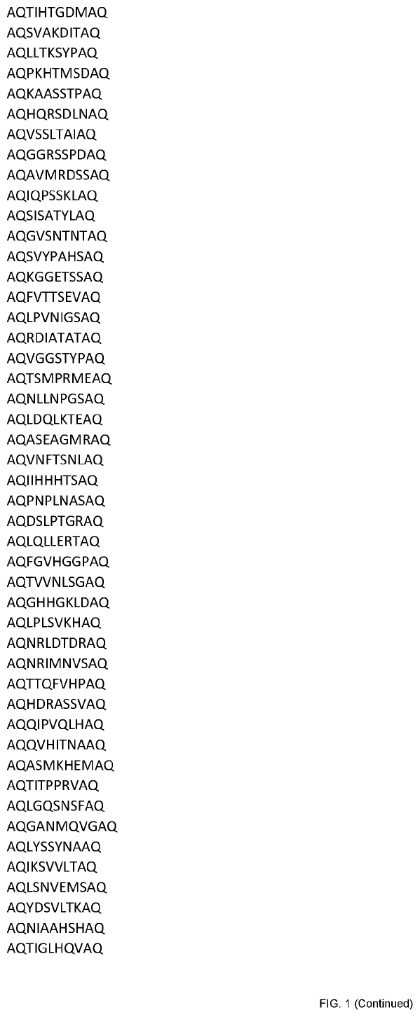

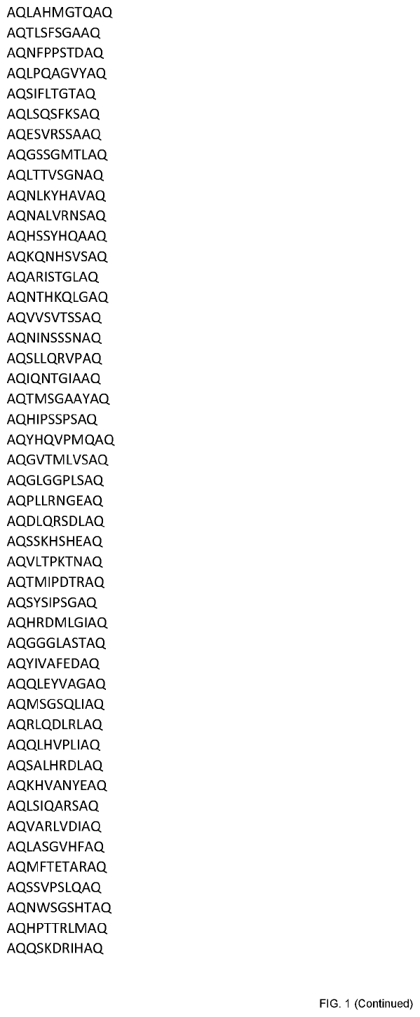

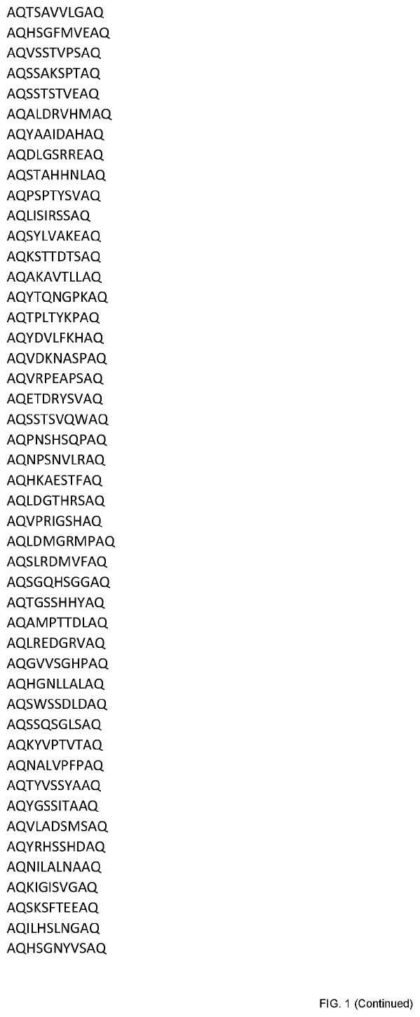

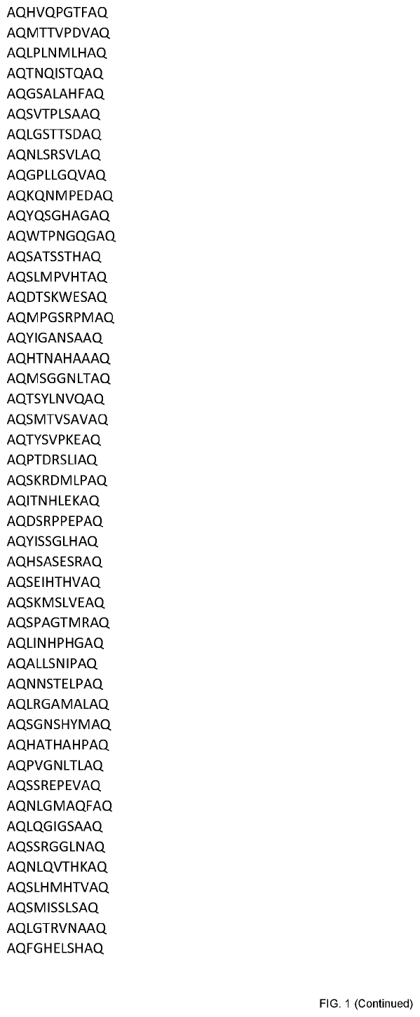

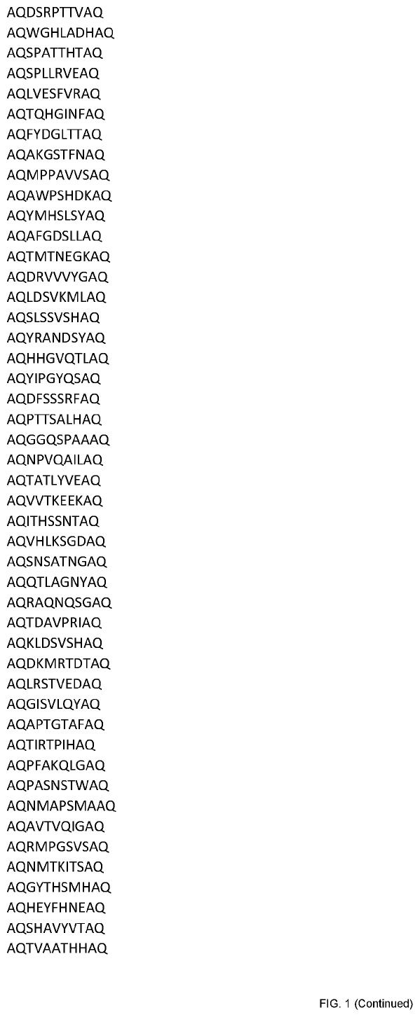

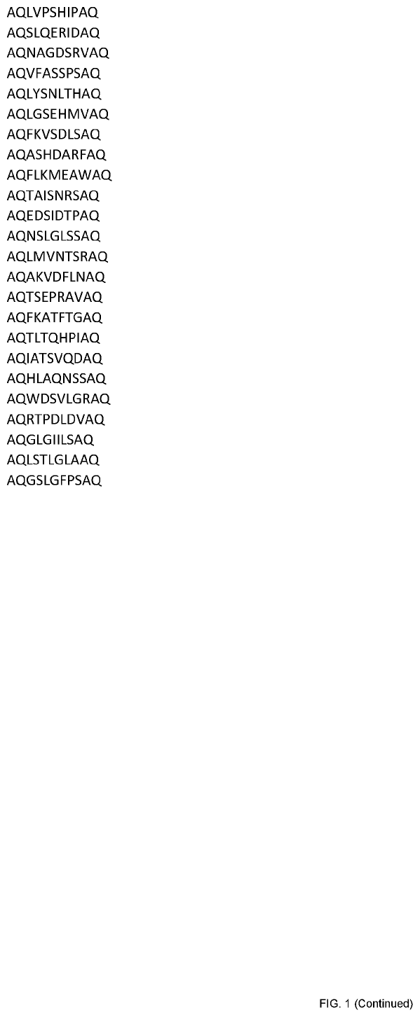

In one aspect, the disclosure provides rAAVs with a peptide insertion and or substitution comprising or consisting of an amino-acid sequence set forth in any one of Tables 1-2, FIG. 1 and/or Formulas I-VIII.

Aspects disclosed herein provide AAV capsids comprising an AAV capsid protein comprising an amino acid sequence of Formula I

X¹-G-G-X²-X³-X⁴-X⁵ (1)

wherein X¹ is an amino acid selected from G, I, K, L and V; X² is an amino acid selected from E, G, H, L, N, S and Q; X³ is an amino acid selected from A, F, I, L, P, T and Y; X⁴ is an amino acid selected from F, L, S and Y; and X⁵ is an amino acid selected from E, H, I, K, P, S and T.

In some embodiments, the AAV capsid protein comprises an amino acid sequence of formula I wherein X¹ is I, K or L. In some embodiments, the AAV capsid protein comprises an amino acid sequence of formula I wherein X¹ is K. In some embodiments, the AAV capsid protein comprises an amino acid sequence of formula I wherein X¹ is I.

In some embodiments, the AAV capsid protein comprises an amino acid sequence of formula I wherein X² is L, H or Q. In some embodiments, the AAV capsid protein comprises an amino acid sequence of formula I wherein X² is L. In some embodiments, the AAV capsid protein comprises an amino acid sequence of formula I wherein X² is H.

In some embodiments, the AAV capsid protein comprises an amino acid sequence of formula I wherein X³ is I, P or Y. In some embodiments, the AAV capsid protein comprises an amino acid sequence of formula I wherein X³ is P. In some embodiments, the AAV capsid protein comprises an amino acid sequence of formula I wherein X³ is Y.

In some embodiments, the AAV capsid protein comprises an amino acid sequence of formula I wherein X⁴ is F or L.

In some embodiments, the AAV capsid protein comprises an amino acid sequence of formula I wherein X⁵ is an I or H.

In some embodiments, the sequence as described in FIG. 1, is selected from KGGLPFI, IGGHYLH, LGGQILI, LGGGFSE, IGGNLLK, KGGETSS, VGGSTYP, and GGGLAST.

In some embodiments, the sequence as described in FIG. 1, is selected from AQKGGLPFIAQ, AQIGGHYLHAQ, AQLGGQILIAQ, AQLGGGFSEAQ, AQIGGNLLKAQ, AQKGGETSSAQ, AQVGGSTYPAQ, and AQGGGLASTAQ.

Aspects disclosed herein provide AAV capsids with greater expression in heart comprising an AAV capsid protein comprising an amino acid sequence of Formula II

R-S-X⁶-X⁷-X⁸-X⁹-X¹⁰ (II)

wherein X⁶ is an amino acid selected from E, G, I, N, Q and T; X⁷ is an amino acid selected from L, H, Q, S, and T; X⁸ is an amino acid selected from D, E, H, P, and S; X⁹ is an amino acid selected from A, F, L, R, S and T; and X¹⁰ is an amino acid selected from E, I, M, S and V.

In some embodiments, the AAV capsid protein comprises an amino acid sequence of formula II wherein X¹⁰ is I, V or M. In some embodiments, the AAV capsid protein comprises an amino acid sequence of formula II wherein X^{V} is M.

In some embodiments, the AAV capsid protein comprises an amino acid sequence of formula II wherein X⁹ is F or L. In some embodiments, the AAV capsid protein comprises an amino acid sequence of formula II wherein X⁹ is F.

In some embodiments, the AAV capsid protein comprises an amino acid sequence of formula II wherein X⁸ is P or H. In some embodiments, the AAV capsid protein comprises an amino acid sequence of formula II wherein X⁸ is P. In some embodiments, the AAV capsid protein comprises an amino acid sequence of formula II wherein X⁸ is H.

In some embodiments, the AAV capsid protein comprises an amino acid sequence of formula II wherein X⁷ is H or L. In some embodiments, the AAV capsid protein comprises an amino acid sequence of formula II wherein X⁷ is H. In some embodiments, the AAV capsid protein comprises an amino acid sequence of formula II wherein X⁷ is L.

In some embodiments, the AAV capsid protein comprises an amino acid sequence of formula II wherein X⁶ is E or G. In some embodiments, the AAV capsid protein comprises an amino acid sequence of formula II wherein X⁶ is E. In some embodiments, the AAV capsid protein comprises an amino acid sequence of formula II wherein X⁷ is G.

In some embodiments, the insertion sequence as described in FIG. 1, is selected from RSGHPFV, RSGLHLM, RSELHFI, RSQTSTE, RSISDRV. RSTTDAS, and RSNQESV.

In some embodiments, the insertion sequence as described in FIG. 1, is selected from AQRSGHPFVAQ, AQRSGLHLMAQ, AQRSELHFIAQ, AQRSQTSTEAQ, AQRSISDRVAQ, AQRSTTDASAQ, and AQRSNQESVAQ.

Aspects disclosed herein provide AAV capsids with greater expression in heart comprising an AAV capsid protein comprising an amino acid sequence of Formula III

R-X¹¹-G-X²-X¹³-X¹⁴-X¹⁵ (III)

wherein X¹¹ is an amino acid selected from A, D, E, I, N, V and S; X¹² is an amino acid selected from E, H, L, M, S and V; X¹³ is an amino acid selected from F, H, L, P, V and Y; X¹⁴ is an amino acid selected from F, L and T; and X¹⁵ is an amino acid selected from D, H, K, L, M, Q, S and V.

In some embodiments, the AAV capsid protein comprises an amino acid sequence of formula III wherein X¹¹ is an amino acid selected from A, D, N and S. In some embodiments, the AAV capsid protein comprises an amino acid sequence of formula III wherein X¹¹ is A. In some embodiments, the AAV capsid protein comprises an amino acid sequence of formula III wherein X¹¹ is D. In some embodiments, the AAV capsid protein comprises an amino acid sequence of formula III wherein X¹¹ is N. In some embodiments, the AAV capsid protein comprises an amino acid sequence of formula III wherein X¹¹ is S.

In some embodiments, the AAV capsid protein comprises an amino acid sequence of formula III wherein X¹² is an amino acid selected from E, H, L, and V. In some embodiments, the AAV capsid protein comprises an amino acid sequence of formula III wherein X² is H. In some embodiments, the AAV capsid protein comprises an amino acid sequence of formula III wherein X¹² is L. In some embodiments, the AAV capsid protein comprises an amino acid sequence of formula III wherein X¹² is E. In some embodiments, the AAV capsid protein comprises an amino acid sequence of formula III wherein X¹² is V.

In some embodiments, the AAV capsid protein comprises an amino acid sequence of formula III wherein X¹³ is an amino acid selected from F, H, P, and Y.

In some embodiments, the AAV capsid protein comprises an amino acid sequence of formula III wherein X⁴ is F or L.

In some embodiments, the AAV capsid protein comprises an amino acid sequence of formula II wherein X⁵ is an amino acid selected from K, L, M and V. In some embodiments, the AAV capsid protein comprises an amino acid sequence of formula III wherein X⁵ is V. In some embodiments, the AAV capsid protein comprises an amino acid sequence of formula III wherein X¹⁵ is I. In some embodiments, the AAV capsid protein comprises an amino acid sequence of formula III wherein X¹⁵ is K. In some embodiments, the AAV capsid protein comprises an amino acid sequence of formula III wherein X¹⁵ is L.

In some embodiments, the insertion sequence as described in FIG. 1, is selected from RSGHPFV, RNGEHFK, RDGHYLL, RNGEFFH, REGMPFL, RAGVHFV, RSGLHLM, RIGVHTS, RIGSVTQ, and RVGSLTD.

Aspects disclosed herein provide AAV capsids with greater expression in the heart comprising an AAV capsid protein comprising an amino acid sequence of Formula IV

X¹⁶-X¹⁷-G-H-X¹⁸-X¹⁹-X²⁰ (IV)

wherein X¹⁶ is an amino acid selected from I, R, and L; X¹⁷ is an amino acid selected from D, G, Q, and S; X¹⁸ is an amino acid selected from I, P and Y; X¹⁹ is an amino acid selected from F, I, L, S and Y; and X²⁰ is an amino acid selected from H, L and V.

In some embodiments, the AAV capsid protein comprises an amino acid sequence of formula IV wherein X¹⁶ is an amino acid selected from I, L and R.

In some embodiments, the AAV capsid protein comprises an amino acid sequence of formula IV wherein X¹⁷ is selected from G, Q and S.

In some embodiments, the AAV capsid protein comprises an amino acid sequence of formula IV wherein X¹⁹ is an amino acid selected from F, I, L, S and Y.

In some embodiments, the AAV capsid protein comprises an amino acid sequence of formula IV wherein X²⁰ is L. In some embodiments, the AAV capsid protein comprises an amino acid sequence of formula IV wherein X²⁰ is H. In some embodiments, the AAV capsid protein comprises an amino acid sequence of formula IV wherein X²⁰ is V.

In some embodiments, the AAV capsid protein comprises an amino acid sequence of formula IV wherein X¹⁸ is I. In some embodiments, the AAV capsid protein comprises an amino acid sequence of formula IV wherein X's is P. In some embodiments, the AAV capsid protein comprises an amino acid sequence of formula IV wherein X¹⁸ is Y.

In some embodiments, the insertion sequence as described in FIG. 1, is selected from RSGHPFV, RDGHYLL, LQGHILL, and IGGHYLH.

In some embodiments, the insertion sequence as described in FIG. 1, is selected from AQRSGHPFVAQ, AQRDGHYLLAQ, AQLQGHILLAQ, and AQIGGHYLHAQ.

Aspects disclosed herein provide AAV capsids with greater expression in the heart comprising an AAV capsid protein comprising an amino acid sequence of Formula V

R-X²¹-X²²-X²³-H-F-X²⁴ (V)

Wherein X²¹ is an amino acid selected from A, N and S; X²² is an amino acid selected from D, E and G; X²³ is an amino acid selected from E, L and V; and X²⁴ is an amino acid selected from I, K, Q and V.

In some embodiments, the AAV capsid protein comprises an amino acid sequence of formula V wherein X²¹ is A. In some embodiments, the AAV capsid protein comprises an amino acid sequence of formula V wherein X²¹ is N. In some embodiments, the AAV capsid protein comprises an amino acid sequence of formula V wherein X²¹ is S.

In some embodiments, the AAV capsid protein comprises an amino acid sequence of formula V wherein X²² is D. In some embodiments, the AAV capsid protein comprises an amino acid sequence of formula V wherein X²² is G. In some embodiments, the AAV capsid protein comprises an amino acid sequence of formula V wherein X²² is E.

In some embodiments, the AAV capsid protein comprises an amino acid sequence of formula V wherein X²³ is E. In some embodiments, the AAV capsid protein comprises an amino acid sequence of formula V wherein X²³ is L. In some embodiments, the AAV capsid protein comprises an amino acid sequence of formula V wherein X²³ is V.

In some embodiments, the AAV capsid protein comprises an amino acid sequence of formula V wherein X²⁴ is Q. In some embodiments, the AAV capsid protein comprises an amino acid sequence of formula V wherein X²⁴ is K. In some embodiments, the AAV capsid protein comprises an amino acid sequence of formula V wherein X²⁴ is V.

In some embodiments, the insertion sequence as described in FIG. 1, is selected from RADLHFQ, RAGVHFV, RNGEHFK, RSELHFI.

In some embodiments, the insertion sequence as described in FIG. 1, is selected from AQRADLHFQAQ, AQRAGVHFVAQ, AQRNGEHFKAQ, and AQRSELHFIAQ.

Aspects disclosed herein provide AAV capsids with greater expression in the heart comprising an AAV capsid protein comprising an amino acid sequence of Formula VI

X²⁵-X²⁶-X²⁷-X²⁸-X²⁹-L-L (VI)

Wherein X²⁵ is an amino acid selected from A, L, R, and Y; X²⁶ is an amino acid selected from Q, E, D, T, K and F; X²⁷ is an amino acid selected from G, Q, and A; X²⁸ is an amino acid selected from H, G, T, V and D; and X²⁹ is an amino acid selected from I, S, T and Y.

In some embodiments, the AAV capsid protein comprises an amino acid sequence of formula VI wherein X²⁵ is L, or R.

In some embodiments, the AAV capsid protein comprises an amino acid sequence of formula VI wherein X²⁶ is Q, E, or D.

In some embodiments, the AAV capsid protein comprises an amino acid sequence of formula VI wherein X²⁷ is G, or Q.

In some embodiments, the AAV capsid protein comprises an amino acid sequence of formula VI wherein X²⁸ is H, or G.

In some embodiments, the AAV capsid protein comprises an amino acid sequence of formula VI wherein X²⁹ is I, or Y.

In some embodiments, the insertion sequence as described in FIG. 1, is selected from LQGHILL, REQGYLL, RDGHYLL, YTGTSLL, AKAVTLL, and AFGDSLL.

In some embodiments, the insertion sequence as described in FIG. 1, is selected from AQLQGHILLAQ, AQREQGYLLAQ, AQRDGHYLLAQ, AQYTGTSLLAQ, AQAKAVTLLAQ, and AQAFGDSLLAQ.

Aspects disclosed herein provide AAV capsids with greater expression in the heart comprising an AAV capsid protein comprising an amino acid sequence of Formula VII

X³⁰-X³¹-X³²-X³³-X³⁴-S-S (VII)

Wherein X³⁰ is an amino acid selected from A, E, F, H, I, K, L, M, N, P, Q, S, T, V and Y; X³¹ is an amino acid selected from F, G, H, I, K, L, M, P, Q, R, S, T, and V; X³² is an amino acid selected from A, G, H, L, P, Q, S and V; X³³ is an amino acid selected from E, G, H, I, L, P, Q, R, S, T and V; and X³⁴ is an amino acid selected from D, G, I, L, M, N, R, S, T and V.

In some embodiments, the AAV capsid protein comprises an amino acid sequence of formula VII wherein X³⁰ is V Q or H.

In some embodiments, the AAV capsid protein comprises an amino acid sequence of formula VII wherein X³¹ is F, L or M.

In some embodiments, the AAV capsid protein comprises an amino acid sequence of formula VII wherein X³² is S, A or Q.

In some embodiments, the AAV capsid protein comprises an amino acid sequence of formula VII wherein X³³ is G, H or S.

In some embodiments, the AAV capsid protein comprises an amino acid sequence of formula VII wherein X³⁴ is G, V or M.

In some embodiments, the insertion sequence as described in FIG. 1, is selected from VFSGVSS, QLQHGSS, HMASMSS, SSLTLSS, SVPSSSS, ITPSVSS, MTVQVSS, SHLSVSS, YPHQTSS, PQGGSSS, FRSSLSS, ISVSISS, ETQRGSS, IHPPSSS, TSAITSS, FKSQSSS, ASHSTSS, AVMRDSS, KGGETSS, VVSVTSS, LISIRSS, NSLGLSS, and HLAQNSS.

In some embodiments, the insertion sequence as described in FIG. 1, is selected from AQVFSGVSSAQ, AQQLQHGSSAQ, AQHMASMSSAQ, AQSSLTLSSAQ, AQSVPSSSSAQ, AQITPSVSSAQ, AQMTVQVSSAQ, AQSHLSVSSAQ, AQYPHQTSSAQ, AQPQGGSSSAQ, AQFRSSLSSAQ, AQISVSISSAQ, AQETQRGSSAQ, AQIHPPSSSAQ, AQTSAITSSAQ, AQFKSQSSSAQ, AQASHSTSSAQ, AQAVMRDSSAQ, AQKGGETSSAQ, AQVVSVTSSAQ, AQLISIRSSAQ, AQNSLGLSSAQ, and AQHLAQNSSAQ.

Aspects disclosed herein provide AAV capsids with greater expression in the heart comprising an AAV capsid protein comprising an amino acid sequence of Formula VIII D-L-X³⁵-X³⁶-X³⁷-X³⁸-X³⁹ (VIII) Wherein X^{3G} is an amino acid selected from G, H, Q, S and T; X³⁶ is an amino acid selected from K, N, P, R and S; X³⁷ is an amino acid selected from H, L, R and S; X³⁸ is an amino acid selected from D, K, R, S, T and V; and X³⁹ is an amino acid selected from A, D, E, L and V.

In some embodiments, the AAV capsid protein comprises an amino acid sequence of formula VIII wherein X³⁵ is H or S.

In some embodiments, the AAV capsid protein comprises an amino acid sequence of formula VIII wherein X³⁶ is K, or R.

In some embodiments, the AAV capsid protein comprises an amino acid sequence of formula VIII wherein X³⁷ is L, or S.

In some embodiments, the AAV capsid protein comprises an amino acid sequence of formula VIII wherein X³⁸ is S, or V.

In some embodiments, the AAV capsid protein comprises an amino acid sequence of formula VIII wherein X³⁹ is A, or L.

In some embodiments, the insertion sequence as described in FIG. 1, is selected from DLSRLS, DLHKSVA, DLGNLKV, DLTPHKD, DLQRSDL, and DLGSRRE.

In some embodiments, the insertion sequence as described in FIG. 1, is selected from AQDLSRLSLAQ, AQDLHKSVAAQ, AQDLGNLKVAQ, AQDLTPHKDAQ, AQDLQRSDLAQ, and AQDLGSRREAQ.

In some embodiments, the insertion sequence is represented by the peptide sequences listed in Tables 1-2.

TABLE 1

| Sequence | Sequence |
|---|---|
| DLSRLSL | AQDLSRLSLAQ |
| KLDPLLM | AQKLDPLLMAQ |
| TPKHLLI | AQTPKHLLIAQ |
| PDQRRTD | AQPDQRRTDAQ |
| RHDPLVM | AQRHDPLVMAQ |
| SHFSGRN | AQSHFSGRNAQ |
| VSAVRDR | AQVSAVRDRAQ |
| VGVPVVL | AQVGVPVVLAQ |
| LLPSNYL | AQLLPSNYLAQ |
| QLQITKE | AQQLQITKEAQ |
| LQVTSVL | AQLQVTSVLAQ |
| VFSGVSS | AQVFSGVSSAQ |

TABLE 2

| Sequence | Sequence |
|---|---|
| KGGLPFI | AQKGGLPFIAQ |
| DLSRLSL | AQDLSRLSLAQ |
| RSGHPFV | AQRSGHPFVAQ |
| RADLHFQ | AQRADLHFQAQ |
| LQGHILL | AQLQGHILLAQ |
| KLDPLLM | AQKLDPLLMAQ |
| IGGHYLH | AQIGGHYLHAQ |
| RAGVHFV | AQRAGVHFVAQ |
| LGGQILI | AQLGGQILIAQ |
| TPKHLLI | AQTPKHLLIAQ |
| RNGEHFK | AQRNGEHFKAQ |
| KDSIPFR | AQKDSIPFRAQ |
| PDQRRTD | AQPDQRRIDAQ |
| RHDPLVM | AQRHDPLVMAQ |
| DNYTRHT | AQDNYTRHTAQ |
| RSGLHLM | AQRSGLHLMAQ |
| TNSVRVV | AQTNSVRVVAQ |

In some aspects, the insertion amino acid sequence is at least 71.4% identical to the amino acid sequence provided in Tables 1-2, FIG. 1 and and/or Formulas I-VII. In some aspects, the insertion amino acid sequence is at least 86.7% identical to the amino acid sequence provided in Tables 1-2, FIG. 1 and and/or Formulas I-VII.

Also disclosed herein are methods and kits for producing therapeutic recombinant AAV (rAAV) particles, as well as methods and pharmaceutical compositions or formulations comprising the rAAV particles, for the treatment of a disease or condition affecting the heart.

Disclosed herein are AAV capsids engineered with increased viral transduction in the heart. The AAV capsids can encapsidate a viral vector with a heterologous nucleic acid encoding, for example, a therapeutic gene expression product. Transduction of the heterologous nucleic acid in the heart can be achieved upon systemic delivery to a subject of the AAV capsid of the present disclosure encapsidating a heterologous nucleic acid. The AAV capsids disclosed herein are advantageous for many applications of gene therapy to treat human disease, including, but not limited to, disorders of the cardiovascular system.

The recombinant AAV vectors comprising a nucleic acid sequence encoding the AAV capsid proteins of the present disclosure as also provided herein. For example, the viral vectors of the present disclosure comprise a nucleic acid sequence comprising the AAV viral Cap (Capsid) encoding VP1, VP2, and VP3, at least one of which is modified to produce the AAV capsid proteins of the present disclosure. The recombinant AAV vector provided can be derived from an AAV serotype (e.g., AAV9) or a variant AAV serotype including an insertion of the present invention.

### AAV Capsids

Provided herein are modified adeno-associated (AAV) virus capsid compositions useful for integrating a transgene into a target cell or environment (in a subject when they are administered systemically to the subject.

An rAAV comprises an AAV capsid that can be engineered to encapsidate a heterologous nucleic acid (e.g., therapeutic nucleic acid, gene editing machinery). The AAV capsid is made up of three AAV capsid protein monomers, VP1, VP2, and VP3. Sixty copies of these three VP proteins interact in a 1:1:10 ratio to form the viral capsid. VP1 covers the whole of VP2 protein in addition to a ˜137 amino acid N-terminal region (VP1u), VP2 covers the whole of VP3 in addition to ˜65 amino acid N-terminal region (VP1/2 common region). The three capsid proteins share a conserved amino acid sequence of VP3, which in some cases is the region beginning at amino acid position 138 (e.g., AA139-736).

While not wishing to be bound by theory, it is understood that a parent AAV capsid sequence comprises a VP1 region. In certain embodiments, a parent AAV capsid sequence comprises a VP1, VP2 and/or VP3 region, or any combination thereof. A parent VP1 sequence may be considered synonymous with a parent AAV capsid sequence.

The AAV VP3 structure contains highly conserved regions that are common to all serotypes, a core eight-stranded β-barrel motif (βB-βI) and a small α-helix (αA). The loop regions inserted between the β-strands consist of the distinctive HI loop between β-strands H and I, the DE loop between β-strands D and E, and nine variable regions (VRs), which form the top of the loops. These VRs, such as the AA588 loop, are found on the capsid surface and can be associated with specific functional roles in the AAV life cycle including receptor binding, transduction and antigenic specificity.

In some aspects, the rAAV variant of the present invention comprises an AAV capsid protein having a peptide insertion at the residues corresponding to amino acids 588-589 of the AAV9 native sequence of SEQ ID NO: 1.

The AAV capsids comprise AAV capsid proteins (e.g., VP1, VP2, and VP3), each with an insertion, such as in the 588 loop of a parental AAV capsid protein structure (AAV9 VP1 numbering). The 588 loop contains the site of heparan sulfate binding of AAV2 and is amenable to peptide display. The only known receptors for AAV9 is N-linked terminal galactose and AAV receptor (AAVR), but many indications point toward there being others. Modifications to AAV9 588 loop are shown herein to confer an increased transgene transduction in target in vivo environments.

The present invention provides, in an aspect, a peptide insertion at the AAV 588 loop comprising or consisting of an amino-acid sequence set forth in any one of Tables 1-2, FIG. 1 and and/or Formulas I-VII.

Disclosed herein are AAV capsids comprising AAV capsid proteins with an insertion at the 588 loop that confer a higher transduction cardiac cell types (e.g.). In particular, the AAV capsid proteins disclosed herein enable rAAV-mediated transduction of a heterologous nucleic acid (e.g., transgene) in the heart of a subject. The AAV capsids of the present disclosure may be formulated as a pharmaceutical composition. In addition, the AAV capsids can be isolated and purified to be used for a variety of applications.

In some embodiments, the rAAV capsid of the present disclosure are generated using the methods disclosed herein. In some instances, the rAAV capsid is chimeric. In some instances, the rAAV, or variant AAV protein comprises therein, confer an increase in a localization of the rAAV within the target tissue, as compared to the parental AAV capsid or capsid protein.

### AAV Capsid Proteins

Disclosed herein are recombinant AAV (rAAV) capsids which comprise AAV capsid proteins that are engineered with a modified capsid protein (e.g., VP1, VP2, VP3). In some embodiments, the rAAV capsid proteins of the present disclosure are generated using the methods disclosed herein. In some embodiments, the AAV capsid proteins are used in the methods of delivering a therapeutic nucleic acid (e.g., a transgene) to a subject. In some instances, the rAAV capsid proteins have desired AAV expression rendering them particularly suitable for certain therapeutic applications, e.g., the treatment of a disease or disorder in a subject such as those disclosed herein.

The rAAV capsid proteins are engineered for optimized expression in the heart of a subject upon systemic administration of the rAAV to the subject. The rAAV capsid proteins are engineered to include the insertions provided in Tables 1-2, FIG. 1 and and/or Formulas I-VII. The rAAV capsid proteins including the insertions provided in Tables 1-2, FIG. 1 and and/or Formulas I-VII are engineered to achieve efficient transduction of an encapsidated transgene. In particular, the rAAV capsid proteins have increased expression in the heart of a subject.

The engineered AAV capsid proteins described herein have, in some cases, an insertion of an amino acid that is heterologous to the parental AAV capsid protein at amino acid positions in the 588 loop. In some embodiments, the amino acid is not endogenous to the parental AAV capsid protein at the amino acid position of the insertion. The amino acid may be a naturally occurring amino acid in the same or equivalent amino acid position as the insertion of the substitution in a different AAV capsid protein.

Generally, the insertion comprises a five-, six-, or seven-amino acid sequence (5-mer, 6-mer, or 7-mer, respectively) that is inserted or substituted at the 588 loop in a parental AAV capsid protein. Aspects provided herein provide amino acid insertions comprising seven amino acid polymer (7-mer) inserted at AA588-589, and may additionally include a substitution of one or two amino acids at amino acid positions flanking the 7-mer sequence (e.g., AA587-588 and/or AA589-590) to produce an eleven amino acid polymer (11-mer) at the 588 loop of a parental AAV capsid protein. The 7-mers described herein were advantageously generated using polymerase chain reaction (PCR) with degenerate primers, where each of the seven amino acids is encoded by a deoxyribose nucleic acid (DNA) sequence N-N-K. “N” is any of the four DNA nucleotides and K is guanine (G) or thymine (T). This method of generating random 7-mer amino acid sequences enables 1.28 billion possible combinations at the protein level.

The rAAV capsid proteins of the present disclosure comprise an insertion of an amino acid in an amino acid sequence of an AAV capsid protein. The AAV capsid, from which an engineered AAV capsid protein of the present disclosure is produced, is referred to as a “parental” AAV capsid. The complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562.

In some cases, the parental AAV is derived from an AAV with a serotype selected from AAV1, AAV2, AAV3, AAV4, AAV5, AAV6, AAV7, AAV8, AAV9, AAV10, AAV11 and AAV12. The AAV capsid protein that is “derived” from another may be a variant AAV capsid protein. A variant may include, for example, a heterologous amino acid in an amino acid sequence of the AAV capsid protein. The heterologous amino acid may be non-naturally occurring in the AAV capsid protein. The heterologous amino acid may be naturally occurring in a different AAV capsid protein. In some instances, the parental AAV capsid is described in US Pat Publication 2020/0165576 and U.S. Pat. App. Ser. No. 62/832,826 and PCT/US20/20778; the content of each of which is incorporated herein.

In some instances, the parental AAV is AAV9. In some instances, the amino acid sequence of the AAV9 capsid protein comprises SEQ ID NO: 1. The amino acid sequence of AAV9 VP1 capsid protein (>tr|Q6JC40|Q6JC40_9VIRU Capsid protein VP1 OS=Adeno-associated virus 9 OX=235455 GN=cap PE=1 SV=1) is provided in SEQ ID NO: 1 (MAADGYLPDWLEDNLSEGIREWWALKPGAPQPKANQQHQDNARGLVLPGYKYLGPGNG LDKGEPVNAADAAALEHDKAYDQQLKAGDNPYLKYNHADAEFQERLKEDTSFGGNLGRA VFQAKKRLLEPLGLVEEAAKTAPGKKRPVEQSPQEPDSSAGIGKSGAQPAKKRLNFGQTGD TESVPDPQPIGEPPAAPSGVGSLTMASGGGAPVADNNEGADGVGSSSGNWHCDSQWLGDR VITTSTRTWALPTYNNHLYKQISNSTSGGSSNDNAYFGYSTPWGYFDFNRFHCHFSPRDWQ RLINNNWGFRPKRLNFKLFNIQVKEVTDNNGVKTIANNLTSTVQVFTDSDYQLPYVLGSAH EGCLPPFPADVFMIPQYGYLTLNDGSQAVGRSSFYCLEYFPSQMLRTGNNFQFSYEFENVPF HSSYAHSQSLDRLMNPLIDQYLYYLSKTINGSGQNQQTLKFSVAGPSNMAVQGRNYIPGPS YRQQRVSTTVTQNNNSEFAWPGASSWALNGRNSLMNPGPAMASHKEGEDRFFPLSGSLIF GKQGTGRDNVDADKVMITNEEEIKTTNPVATESYGQVATNHQSAQAQAQTGWVQNQGIL PGMVWQDRDVYLQGPIWAKIPHTDGNFHPSPLMGGFGMKHPPPQILIKNTPVPADPPTAFN KDKLNSFITQYSTGQVSVEIEWELQKENSKRWNPEIQYTSNYYKSNNVEFAVNTEGVYSEP RPIGTRYLTRNL). In some instances, the parental AAV capsid protein sequence is 70%, 75%, 80%, 85%, 90%, 91%, 92%, 93%, 94%, 95%, 96%, 97%, 98%, 99%, or 100% homologous to SEQ ID NO: 1.

AAV capsid proteins from native AAV serotypes, such as AAV9, with tropisms including the liver activate the innate immune response, which in come cases causes a severe inflammatory response in a subject, which can lead to multi-organ failure. By improving transduction of a native AAV serotype for a target in vivo tissue (e.g., heart), the rAAV particles of the present disclosure may reduce the unwanted side effects.

In some instances, the parental AAV capsid protein comprises the entire VP1 region provided in SEQ ID NO: 1 (e.g., amino acids 1-736). In some instances, the parental AAV capsid protein comprises amino acids 217-736 in SEQ ID NO: 1, which is the common region found in VP1, VP2 and VP3 AAV9 capsid proteins. In some instances, the AAV capsid protein comprises amino acids 64-736 in SEQ ID NO: 1, which is the common region found in VP1 and VP2. The parental AAV capsid protein sequence may comprise amino acids selected from 1-736, 10-736, 20-736, 30-736, 40-736, 50-736, 60-736, 70-736, 80-736, 90-736, 100-736, 110-736, 120-736, 130-736, 140-736, 150-736, 160-736, 170-736, 180-736, 190-736, 200-736, 210-736, 220-736, 230-736, 240-736, 250-736, 260-736, 270-736, 280-736, 290-736, 300-736, 310-736, 320-736, 330-736, 340-736, 350-736, 360-736, 370-736, 380-736, 390-736, 400-736, 410-736, 420-736, 430-736, 440-736, and 450-736, from SEQ ID NO: 1. In some aspects, the rAAV variant comprises an AAV capsid protein comprising an amino acid sequence that is at least 98% identical to amino acid 217 to amino acid 736 of SEQ ID NO: 1. In some instances, the amino acid insertion is at a three (3)-fold axis of symmetry of a corresponding parental AAV capsid protein.

Disclosed herein are insertions of an amino acid sequence in an AAV capsid protein. Where the sequence numbering designation “588-589” is noted for AAV9, for example AAV VP1, the invention also includes insertions in similar locations in the other AAV serotypes. As used herein, “AA588-589” indicates that the insertion of the amino acid (or amino acid sequence) is immediately after an amino acid (AA) at position 588 and immediately before an AA at position 589 within an amino acid sequence of a parental AAV VP capsid protein (VP1 numbering). Amino acids 587-591 include a motif comprising “AQAQA” as set forth in SEQ ID NO: 1. Exemplary AAV capsid protein sequences are provided in Table 3. For example, KGGLPFI is inserted at AA588-589 in an AAV9 capsid amino acid sequence, and provides variant A. It is envisioned that the sequences disclosed herein (Tables 1-2, FIG. 1 and Formulas I-VII) may be inserted at AA588-589 in an amino acid sequence of a parental AAV9 capsid protein or at AA587-590 (replacing amino acids AA587-590), a variant thereof, or equivalent amino acid position of a parental AAV of a different serotype (e.g., AAV 1, AAV2, AAV3, and the like). In any AAV capsid protein sequence disclosed herein, the amino acid at position 449

TABLE 3
Exemplary AAV Capsid Protein Sequences

| Identifier | Sequence |
|---|---|
| Variant A | MAADGYLPDWLEDNLSEGIREWWALKPGAPQPKANQQHQ |
| | DNARGLVLPGYKYLGPGNGLDKGEPVNAADAAALEHDKA |
| | YDQQLKAGDNPYLKYNHADAEFQERLKEDTSFGGNLGRAV |
| | FQAKKRLLEPLGLVEEAAKTAPGKKRPVEQSPQEPDSSAGIG |
| | KSGAQPAKKRLNFGQTGDTESVPDPQPIGEPPAAPSGVGSLT |
| | MASGGGAPVADNNEGADGVGSSSGNWHCDSQWLGDRVIT |
| | TSTRTWALPTYNNHLYKQISNSTSGGSSNDNAYFGYSTPWG |
| | YFDFNRFHCHFSPRDWQRLINNNWGFRPKRLNFKLFNIQVK |
| | EVTDNNGVKTIANNLTSTVQVFTDSDYQLPYVLGSAHEGCL |
| | PPFPADVFMIPQYGYLTLNDGSQAVGRSSFYCLEYFPSQMLR |
| | TGNNFQFSYEFENVPFHSSYAHSQSLDRLMNPLIDQYLYYLS |
| | RTINGSGQNQQTLKFSVAGPSNMAVQGRNYIPGPSYRQQRV |
| | STTVTQNNNSEFAWPGASSWALNGRNSLMNPGPAMASHKE |
| | GEDRFFPLSGSLIFGKQGTGRDNVDADKVMITNEEEIKTTNP |
| | VATESYGQVATNHQSAQKGGLPFIAQAQTGWVQNQGILP |
| | GMVWQDRDVYLQGPIWAKIPHTDGNFHPSPLMGGFGMKH |
| | PPPQILIKNTPVPADPPTAFNKDKLNSFITQYSTGQVSVEIEW |
| | ELQKENSKRWNPEIQYTSNYYKSNNVEFAVNTEGVYSEPRP |
| | IGTRYLTRNL |
| Variant B | MAADGYLPDWLEDNLSEGIREWWALKPGAPQPKANQQHQ |
| | DNARGLVLPGYKYLGPGNGLDKGEPVNAADAAALEHDKA |
| | YDQQLKAGDNPYLKYNHADAEFQERLKEDTSFGGNLGRAV |
| | FQAKKRLLEPLGLVEEAAKTAPGKKRPVEQSPQEPDSSAGIG |
| | KSGAQPAKKRLNFGQTGDTESVPDPQPIGEPPAAPSGVGSLT |
| | MASGGGAPVADNNEGADGVGSSSGNWHCDSQWLGDRVIT |
| | TSTRTWALPTYNNHLYKQISNSTSGGSSNDNAYFGYSTPWG |
| | YFDFNRFHCHFSPRDWQRLINNNWGFRPKRLNFKLFNIQVK |
| | EVTDNNGVKTIANNLTSTVQVFTDSDYQLPYVLGSAHEGCL |
| | PPFPADVFMIPQYGYLTLNDGSQAVGRSSFYCLEYFPSQMLR |
| | TGNNFQFSYEFENVPFHSSYAHSQSLDRLMNPLIDQYLYYLS |
| | RTINGSGQNQQTLKFSVAGPSNMAVQGRNYIPGPSYRQQRV |
| | STTVTQNNNSEFAWPGASSWALNGRNSLMNPGPAMASHKE |
| | GEDRFFPLSGSLIFGKQGTGRDNVDADKVMITNEEEIKTTNP |
| | VATESYGQVATNHQSAQDLSRLSLAQAQTGWVQNQGILPG |
| | MVWQDRDVYLQGPIWAKIPHTDGNFHPSPLMGGFGMKHPP |
| | PQILIKNTPVPADPPTAFNKDKLNSFITQYSTGQVSVEIEWEL |
| | QKENSKRWNPEIQYTSNYYKSNNVEFAVNTEGVYSEPRPIG |
| | TRYLTRNL |
| Variant C | MAADGYLPDWLEDNLSEGIREWWALKPGAPQPKANQQHQ |
| | DNARGLVLPGYKYLGPGNGLDKGEPVNAADAAALEHDKA |
| | YDQQLKAGDNPYLKYNHADAEFQERLKEDTSFGGNLGRAV |
| | FQAKKRLLEPLGLVEEAAKTAPGKKRPVEQSPQEPDSSAGIG |
| | KSGAQPAKKRLNFGQTGDTESVPDPQPIGEPPAAPSGVGSLT |
| | MASGGGAPVADNNEGADGVGSSSGNWHCDSQWLGDRVIT |
| | TSTRTWALPTYNNHLYKQISNSTSGGSSNDNAYFGYSTPWG |
| | YFDFNRFHCHFSPRDWQRLINNNWGFRPKRLNFKLFNIQVK |
| | EVTDNNGVKTIANNLTSTVQVFTDSDYQLPYVLGSAHEGCL |
| | PPFPADVFMIPQYGYLTLNDGSQAVGRSSFYCLEYFPSQMLR |
| | TGNNFQFSYEFENVPFHSSYAHSQSLDRLMNPLIDQYLYYLS |
| | RTINGSGQNQQTLKFSVAGPSNMAVQGRNYIPGPSYRQQRV |
| | STTVTQNNNSEFAWPGASSWALNGRNSLMNPGPAMASHKE |
| | GEDRFFPLSGSLIFGKQGTGRDNVDADKVMITNEEEIKTTNP |
| | VATESYGQVATNHQSAQRSGHPFVAQAQTGWVQNQGILP |
| | GMVWQDRDVYLQGPIWAKIPHTDGNFHPSPLMGGFGMKH |
| | PPPQILIKNTPVPADPPTAFNKDKLNSFITQYSTGQVSVEIEW |
| | ELQKENSKRWNPEIQYTSNYYKSNNVEFAVNTEGVYSEPRP |
| | IGTRYLTRNL |
| Variant D | MAADGYLPDWLEDNLSEGIREWWALKPGAPQPKANQQHQ |
| | DNARGLVLPGYKYLGPGNGLDKGEPVNAADAAALEHDKA |
| | YDQQLKAGDNPYLKYNHADAEFQERLKEDTSFGGNLGRAV |
| | FQAKKRLLEPLGLVEEAAKTAPGKKRPVEQSPQEPDSSAGIG |
| | KSGAQPAKKRLNFGQTGDTESVPDPQPIGEPPAAPSGVGSLT |
| | MASGGGAPVADNNEGADGVGSSSGNWHCDSQWLGDRVIT |
| | TSTRTWALPTYNNHLYKQISNSTSGGSSNDNAYFGYSTPWG |
| | YFDFNRFHCHFSPRDWQRLINNNWGFRPKRLNFKLFNIQVK |
| | EVTDNNGVKTIANNLTSTVQVFTDSDYQLPYVLGSAHEGCL |
| | PPFPADVFMIPQYGYLTLNDGSQAVGRSSFYCLEYFPSQMLR |
| | TGNNFQFSYEFENVPFHSSYAHSQSLDRLMNPLIDQYLYYLS |
| | RTINGSGQNQQTLKFSVAGPSNMAVQGRNYIPGPSYRQQRV |
| | STTVTQNNNSEFAWPGASSWALNGRNSLMNPGPAMASHKE |
| | GEDRFFPLSGSLIFGKQGTGRDNVDADKVMITNEEEIKTTNP |
| | VATESYGQVATNHQSAQRADLHFQAQAQTGWVQNQGILP |
| | GMVWQDRDVYLQGPIWAKIPHTDGNFHPSPLMGGFGMKH |
| | PPPQILIKNTPVPADPPTAFNKDKLNSFITQYSTGQVSVEIEW |
| | ELQKENSKRWNPEIQYTSNYYKSNNVEFAVNTEGVYSEPRP |
| | IGTRYLTRNL |
| Variant E | MAADGYLPDWLEDNLSEGIREWWALKPGAPQPKANQQHQ |
| | DNARGLVLPGYKYLGPGNGLDKGEPVNAADAAALEHDKA |
| | YDQQLKAGDNPYLKYNHADAEFQERLKEDTSFGGNLGRAV |
| | FQAKKRLLEPLGLVEEAAKTAPGKKRPVEQSPQEPDSSAGIG |
| | KSGAQPAKKRLNFGQTGDTESVPDPQPIGEPPAAPSGVGSLT |
| | MASGGGAPVADNNEGADGVGSSSGNWHCDSQWLGDRVIT |
| | TSTRTWALPTYNNHLYKQISNSTSGGSSNDNAYFGYSTPWG |
| | YFDFNRFHCHFSPRDWQRLINNNWGFRPKRLNFKLFNIQVK |
| | EVTDNNGVKTIANNLTSTVQVFTDSDYQLPYVLGSAHEGCL |
| | PPFPADVFMIPQYGYLTLNDGSQAVGRSSFYCLEYFPSQMLR |
| | TGNNFQFSYEFENVPFHSSYAHSQSLDRLMNPLIDQYLYYLS |
| | RTINGSGQNQQTLKFSVAGPSNMAVQGRNYIPGPSYRQQRV |
| | STTVTQNNNSEFAWPGASSWALNGRNSLMNPGPAMASHKE |
| | GEDRFFPLSGSLIFGKQGTGRDNVDADKVMITNEEEIKTTNP |
| | VATESYGQVATNHQSAQLQGHILLAQAQTGWVQNQGILP |
| | GMVWQDRDVYLQGPIWAKIPHTDGNFHPSPLMGGFGMKH |
| | PPPQILIKNTPVPADPPTAFNKDKLNSFITQYSTGQVSVEIEW |
| | ELQKENSKRWNPEIQYTSNYYKSNNVEFAVNTEGVYSEPRP |
| | IGTRYLTRNL |
| Variant F | MAADGYLPDWLEDNLSEGIREWWALKPGAPQPKANQQHQ |
| | DNARGLVLPGYKYLGPGNGLDKGEPVNAADAAALEHDKA |
| | YDQQLKAGDNPYLKYNHADAEFQERLKEDTSFGGNLGRAV |
| | FQAKKRLLEPLGLVEEAAKTAPGKKRPVEQSPQEPDSSAGIG |
| | KSGAQPAKKRLNFGQTGDTESVPDPQPIGEPPAAPSGVGSLT |
| | MASGGGAPVADNNEGADGVGSSSGNWHCDSQWLGDRVIT |
| | TSTRTWALPTYNNHLYKQISNSTSGGSSNDNAYFGYSTPWG |
| | YFDFNRFHCHFSPRDWQRLINNNWGFRPKRLNFKLFNIQVK |
| | EVTDNNGVKTIANNLTSTVQVFTDSDYQLPYVLGSAHEGCL |
| | PPFPADVFMIPQYGYLTLNDGSQAVGRSSFYCLEYFPSQMLR |
| | TGNNFQFSYEFENVPFHSSYAHSQSLDRLMNPLIDQYLYYLS |
| | RTINGSGQNQQTLKFSVAGPSNMAVQGRNYIPGPSYRQQRV |
| | STTVTQNNNSEFAWPGASSWALNGRNSLMNPGPAMASHKE |
| | GEDRFFPLSGSLIFGKQGTGRDNVDADKVMITNEEEIKTTNP |
| | VATESYGQVATNHQSAQKLDPLLMAQAQTGWVQNQGILP |
| | GMVWQDRDVYLQGPIWAKIPHTDGNFHPSPLMGGFGMKH |
| | PPPQILIKNTPVPADPPTAFNKDKLNSFITQYSTGQVSVEIEW |
| | ELQKENSKRWNPEIQYTSNYYKSNNVEFAVNTEGVYSEPRP |
| | IGTRYLTRNL |

The insertions described herein may, in some cases, comprise a 7-mer insertion at AA588-589. It is envisioned that any 7-mer insertion disclosed herein in addition to a substitution with any amino acid at amino acid positions 587-590 [AQAQ] may comprise an 11-mer.

Disclosed herein are AAV capsid proteins with an insertion described above in a parental AAV capsid protein that confers an increased transduction in the heart in a subject, even when delivered systemically. One of the many advantages of the AAV capsid proteins described herein is their ability to target tissue and cells within the heart.

In some instances, the AAV capsid protein comprises an insertion of at least or about seven, eight, nine, ten or eleven amino acids of an amino acid sequence of Tables 1-2, FIG. 1 and Formulas I-VII at an amino acid position 588-589 or at 587-590 in a parental AAV9 capsid protein (SEQ ID NO: 1). In some cases, the AAV capsid protein has an increased viral transduction enrichment in heart.

The rAAV capsid proteins of the present disclosure may also have a substitution of an amino acid sequence at amino acid position 452-458 in a parental AAV9 capsid protein, or variant thereof, as described in WO2020068990. In some embodiments, the substitution of the amino acid sequence comprises KDNTPGR at amino acid position 452-458 in the parental AAV9 capsid protein. In some embodiments, the substitution of the amino acid sequence comprises DGAATKN at amino acid position 452-458 in the parental AAV9 capsid protein.

The rAAV capsid proteins described herein may be isolated and purified. The AAV may be isolated and purified by methods standard in the art such as by column chromatography, iodixanol gradients, or cesium chloride gradients. Methods for purifying AAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

In addition, the AAV capsid proteins disclosed herein, either isolated and purified, or not, may be formulated into a pharmaceutical formulation, which in some cases, further comprises a pharmaceutically acceptable carrier.

The rAAV capsid protein can be conjugated to a nanoparticle, a second molecule, or a viral capsid protein. In some cases, the nanoparticle or viral capsid protein would encapsidate the therapeutic nucleic acid described herein. In some instances, the second molecule is a therapeutic agent, e.g., a small molecule, antibody, antigen-binding fragment, peptide, or protein, such as those described herein.

“Percent Identity” is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see: Henikoff and Henikoff, (1989) Proc. Natl. Acad. Sci. USA 89: 10915).

Sequence identity/similarity values provided herein can refer to the value obtained using the BLAST+ 2.5.0 suite of programs using default settings (blast.ncbi.nlm.nih.gov) (Camacho, C., et al. (2009) BLAST+: architecture and applications. BMC Bioinformatics 10:421).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17: 149-63) and XNU (Ci-ayerie and States (1993) Comput. Chem. 17: 191-201) low-complexity filters can be employed alone or in combination.

The terms “substantial identity” and “substantially identical” indicate that a polypeptide or nucleic acid comprises a sequence with between 55-100% sequence identity to a reference sequence, with at least 55% sequence identity, or at least 60%, or at least 65%, or at least 70%, or at least 75%, or at least 80%, or at least 85%, or at least 90%, or at least 95%, or at least 99% sequence identity or any percentage of value within the range of 55-100% sequence identity relative to the reference sequence. The percent sequence identity may occur over a specified comparison window. Optimal alignment may be ascertained or conducted using the homology alignment algorithm of Needleman and Wunsch, supra.

For example, the insertion sequences may include, but are not limited to, sequences that are not exactly the same as the sequences disclosed herein, but which have, in addition to the substitutions explicitly described for various sequences listed herein, additional substitutions of amino acid residues which substantially do not impair the activity or properties of the sequences described herein, such as those predicted by homology software e.g. BLOSUM62 matrices.

### AAV Particles

The rAAV particles with the insertion sequences described herein have an increased transduction enrichment in the heart. In some instances, the increased transduction enrichment comprises a 1-fold, 2-fold, 3-fold, 4-fold, 5-fold, 6-fold, 7-fold, 8-fold, 9-fold or 10-fold increase, or more. In some instances, the increased transduction enrichment is at least 1-fold. In some instances, the increased transduction enrichment is at least 2-fold. In some instances, the increased transduction enrichment is at least 4-fold.

The rAAV particles with the insertion sequences described herein have an increased expression enrichment in the heart. Detecting whether a rAAV possesses more or less specificity for a target in vivo environment, includes measuring a level of gene expression product (e.g., RNA or protein) expressed from the heterologous nucleic acid encapsidated by the rAAV in a tissue sample obtained from a subject. Suitable methods for measuring expression of a gene expression product include next-generation sequencing (NGS) and quantitative polymerase chain reaction (qPCR).

The increased expression in the heart is represented by the enrichment values provided in FIG. 1.

### Heterologous Nucleic Acids

Disclosed herein are therapeutic nucleic acids useful for the treatment or prevention of a disease or condition, or symptom of the disease or condition. In some embodiments, the therapeutic nucleic acids encode a therapeutic gene expression product. Non-limiting examples of gene expression products include proteins, polypeptides, peptides, enzymes, antibodies, antigen binding fragments, nucleic acid (RNA, DNA, antisense oligonucleotide, siRNA, and the like), and gene editing components, for use in the treatment, prophylaxis, and/or amelioration of the disease or disorder, or symptoms of the disease or disorder. In some instances, the therapeutic nucleic acids are placed in an organism, cell, tissue or organ of a subject by way of a rAAV, such as those disclosed herein.

Disclosed herein are rAAVs, each comprising a viral vector (e.g., a single stranded DNA molecule (ssDNA)). In some instances, the viral vector comprises two inverted terminal repeat (ITR) sequences that are about 145 bases each, flanking a transgene. In some embodiments, the transgene comprises a therapeutic nucleic acid, and in some cases, a promoter in cis with the therapeutic nucleic acid in an open reading frame (ORF). The promoter is capable of initiating transcription of therapeutic nucleic acid in the nucleus of the target cell. The ITR sequences can be from any AAV serotype. Non-limiting examples of AAV serotypes include AAV1, AAV2, AAV3, AAV4, AAV5, AAV6, AAV7, AAV8, AAV9, AAV10, AAV11, and AAV12. In some cases, an ITR is from AAV2. In some cases, an ITR is from AAV9.

Disclosed herein are transgenes that can comprise any number of nucleotides. In some cases, a transgene can comprise less than about 100 nucleotides. In some cases, a transgene can comprise at least about 100 nucleotides. In some cases, a transgene can comprise at least about 200 nucleotides. In some cases, a transgene can comprise at least about 300 nucleotides. In some cases, a transgene can comprise at least about 400 nucleotides. In some cases, a transgene can comprise at least about 500 nucleotides. In some cases, a transgene can comprise at least about 1000 nucleotides. In some cases, a transgene can comprise at least about 5000 nucleotides. In some cases, a transgene can comprise between about 500 and about 5000 nucleotides. In some cases, a transgene comprises about 5000 nucleotides. In any of the cases disclosed herein, the transgene can comprise DNA, RNA, or a hybrid of DNA and RNA. In some cases, the transgene can be single stranded. In some cases, the transgene can be double stranded.

Disclosed herein are transgenes useful for modulating the expression or activity of a target gene or gene expression product thereof. In some instances, the transgene is encapsidated by an rAAV capsid protein of an rAAV particle described herein. In some instances, the rAAV particle is delivered to a subject to treat a disease or condition disclosed herein in the subject. In some instances, the delivery is systemic.

The transgenes disclosed herein are useful for expressing an endogenous gene at a level similar to that of a healthy or normal individual. This is particularly useful in the treatment of a disease or condition related to the underexpression, or lack of expression, of a gene expression product. In some embodiments, the transgenes disclosed herein are useful for overexpressing an endogenous gene, such that an expression level of the endogenous gene is above the expression level of a healthy or normal individual. Additionally, transgenes can be used to express exogenous genes (e.g., active agent such as an antibody, peptide, nucleic acid, or gene editing components). In some embodiments, the therapeutic gene expression product is capable of altering, enhancing, increasing, or inducing the activity of one or more endogenous biological processes in the cell. In some embodiments, the transgenes disclosed herein are useful for reducing expression of an endogenous gene, for example, a dominant negative gene. In some embodiments, the therapeutic gene expression product is capable of altering, inhibiting, reducing, preventing, eliminating, or impairing the activity of one or more endogenous biological processes in the cell. In some aspects, the increase of gene expression refers to an increase by at least about 20%, 30%, 40%, 50%, 60%, 70%, 80%, 85%, 90%, 95% and 100%. In one aspect, the protein product of the targeted gene may be increased by at least about 20%, 30%, 40%, 50%, 60%, 70%, 80%, 85%, 90%, 95% and 100%. In some aspects, the decrease of gene expression refers to an increase by at least about 20%, 30%, 40%, 50%, 60%, 70%, 80%, 85%, 90%, 95% and 100%. In one aspect, the protein product of the targeted gene may be decreased by at least about 20%, 30%, 40%, 50%, 60%, 70%, 80%, 85%, 90%, 95% and 100%.

When endogenous sequences (endogenous or part of a transgene) are expressed with a transgene, the endogenous sequences can be full-length sequences (wild-type or mutant) or partial sequences. The endogenous sequences can be functional. Non-limiting examples of the function of these full length or partial sequences include increasing the serum half-life of the polypeptide expressed by a transgene (e.g., therapeutic gene) and/or acting as a carrier.

A transgene can be inserted into an endogenous gene such that all, some or none of the endogenous gene is expressed. For example, a transgene as described herein can be inserted into an endogenous locus such that some (N-terminal and/or C-terminal to a transgene) or none of the endogenous sequences are expressed, for example as a fusion with a transgene. In other cases, a transgene (e.g., with or without additional coding sequences of the endogenous gene) is integrated into any endogenous locus, for example a safe-harbor locus. For example, a Frataxin (FXN) transgene can be inserted into an endogenous FXN gene. A transgene can be inserted into any gene, e.g., the genes as described herein.

At least one advantage of the present disclosure is that virtually any therapeutic nucleic acid may be used to express any therapeutic gene expression product. In some instances, the therapeutic gene expression product is a therapeutic protein or a peptide (e.g., antibody, antigen-binding fragment, peptide, or protein). In one embodiment the protein encoded by the therapeutic nucleic acid is between 50-2000 amino acids in length. In some embodiments the protein encoded is between 50-16000 amino acids in length. In some embodiments the protein encoded is between 50-1500 amino acids in length. In some embodiments the protein encoded is between 50-1000 amino acids in length. In some embodiments the protein encoded is between 50-800 amino acids in length. In some embodiments the protein encoded is between 50-600 amino acids in length. In some embodiments the protein encoded is between 50-400 amino acids in length. In some embodiments the protein encoded is between 50-200 amino acids in length. In some embodiments the protein encoded is between 50-100 amino acids in length. In some embodiments the peptide encoded is between 4-50 amino acids in length. In some embodiments, the protein encoded is a tetrapeptide, a pentapeptide, a hexapeptide, a heptapeptide, an octapeptide, a nonapeptide, or a decapeptide. In some embodiments, the protein encoded comprises a peptide of 2-30 amino acids, such as for example 5-30, 10-30, 2-25, 5-25, 10-25, or 10-20 amino acids. In some embodiments, the protein encoded comprises a peptide of at least 11, 12, 13, 14, 15, 17, 20, 25 or 30 amino acids, or a peptide that is no longer than 50 amino acids, e.g. no longer than 35, 30, 25, 20, 17, 15, 14, 13, 12, 11 or 10 amino acids.

Non-limiting examples of therapeutic protein or peptides include an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinsase inhibitor, a proteolytic protein, a proteolytic protein inhibitor, a semaphoring, a semaphorin receptor, a serotonin transport protein, a serotonin uptake inhibitor, a serotonin receptor, a serpin, a serpin receptor, and a tumor suppressor. In certain embodiments, the therapeutic protein or peptide is selected from brain-derived neurotrophic factor (BDNF), ciliary neurotrophic factor (CNTF), macrophage colony-stimulating factor (CSF), epidermal growth factor (EGF), fibroblast growth factor (FGF), gonadotropin, interferon-gamma (IFN), insulin-like growth factor 1 (IFG-1), nerve growth factor (NGF), platelet-derived growth factor (PDGF), pigment epithelium-derived factor (PEDF), transforming growth factor (TGF), transforming growth factor-beta (TGF-B), tumor necrosis factor (TNF), vascular endothelial growth factor (VEGF), prolactin, somatotropin, X-linked inhibitor of apoptosis protein 1 (XIAP1), interleukin 1 (IL-1), IL-2, IL-3, IL-4, IL-5, IL-6, IL-7, IL-8, IL-9, IL-10, IL-10, viral IL-10, IL-11, IL-12, IL-13, IL-14, IL-15, IL-16, IL-17, and IL-18.

A therapeutic gene expression product can comprise gene editing components. Non-limiting examples of gene editing components include those required for CRISPR/Cas, artificial site-specific RNA endonuclease (ASRE), zinc finger endonuclease (ZFN), and transcription factor like effector nuclease (TALEN). In a non-limiting example, a subject having Huntington's disease is identified. The subject is then systemically administered a first amount of a rAAV encapsidating a viral vector encoding ZFN engineered to represses the transcription of the Huntingtin (HTT) gene. The rAAV will include a modified AAV capsid protein that includes an amino acid sequence provided in any one of Tables 1-2, FIG. 1 and Formulas I-VII, so as to allow proper targeting of the ZFN to the nervous system, while reducing expression in off-target organs, such as the liver. If needed, the subject is administered a second or third dose of the rAAV, until a therapeutically effective amount of the ZFN is expressed in the subject's nervous system.

A therapeutic nucleic acid can comprise a non-protein coding gene e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components described herein. The non-protein coding gene may also encode a tRNA, rRNA, tmRNA, piRNA, double stranded RNA, snRNA, snoRNA, and/or long non-coding RNA (lncRNA). In some cases, the non-protein coding gene can modulate the expression or the activity of a target gene or gene expression product. For example, the RNAs described herein may be used to inhibit gene expression in the cardiovascular system. In some cases, inhibition of gene expression refers to an inhibition by at least about 20%, 30%, 40%, 50%, 60%, 70%, 80%, 85%, 90%, 95% and 100%. In some cases, the protein product of the targeted gene may be inhibited by at least about 20%, 30%, 40%, 50%, 60%, 70%, 80%, 85%, 90%, 95% and 100%. The gene can be either a wild type gene or a gene with at least one mutation. The targeted protein may be either a wild type protein or a protein with at least one mutation.

A therapeutic nucleic acid can modulate the expression or activity of a gene or gene expression product expressed from the gene that is implicated in a disease or disorder of the heart. For example, the therapeutic nucleic acid, in some cases is a gene or a modified version of the gene described herein. In some instances, the gene or gene expression product is inhibited. In some instances, the gene or gene expression product is enhanced.

In another example, the therapeutic nucleic acid comprises an effector gene expression product such as a gene editing component specific to target a gene therein. Non-limited examples of genes include target gene or gene expression product selected from MYBPC3, MYH7, LMNA, SCN5A, DMD, TAZ, BAG3, DSC2, DSG2, DSP, PKP2, CSRP3, phosphatase inhibitor (1-1) protein, KCNH2, TRPM4, ATP2A2, CACNA1C, DMPK, EPG5, EVC, EVC2, FBN1, NF1, SOS1, NPR1, ERBB4, VIP, MYH7, TNNI3, relaxin and FXN.

In some instances, the gene or gene expression product is inhibited. In some instances, the gene or gene expression product is enhanced.

### AAV Vectors

Aspects disclosed herein comprise plasmid vectors comprising a nucleic acid sequence encoding the AAV capsids and AAV capsid proteins described herein. AAV vectors described herein are useful for the assembly of a rAAV and viral packaging of a heterologous nucleic acid. In addition, an AAV vector may encode a transgene comprising the heterologous nucleic acid.

An AAV vector can comprise a transgene, which in some cases encodes a heterologous gene expression product (e.g., therapeutic gene expression product, recombinant capsid protein, and the like). The transgene is in cis with two inverted terminal repeats (ITRs) flanking the transgene. The transgene may comprise a therapeutic nucleic acid encoding a therapeutic gene expression product. Due to the limited packaging capacity of the rAAV (˜5 kB), in some cases, a longer transgene may be split between two AAV vectors, the first with 3′ splice donor and the second with a 5′ splice acceptor. Upon co-infection of a cell, concatemers form, which are spliced together to express a full-length transgene.

A transgene is generally inserted so that its expression is driven by the endogenous promoter at the integration site, namely the promoter that drives expression of the endogenous gene into which a transgene is inserted. In some instances, a transgene comprises a promoter and/or enhancer, for example a constitutive promoter or an inducible or tissue/cell specific promoter. As a non-limiting example, the promoter may be CMV promoter, a CMV-β-Actin-intron-β-Globin hybrid promoter (CAG), CBA promoter, FRDA or FXN promoter, UBC promoter, GUSB promoter, NSE promoter, Synapsin promoter, MeCP2 promoter, GFAP promoter, H1 promoter, U6 promoter, NFL promoter, NFH promoter, SCN8A promoter, or PGK promoter. As a non-limiting example, promoters can be tissue-specific expression elements include, but are not limited to, human elongation factor 1α-subunit (EF1α), immediate-early cytomegalovirus (CMV), chicken β-actin (CBA) and its derivative CAG, the β glucuronidase (GUSB), and ubiquitin C (UBC). The transgene may include a tissue-specific expression elements for neurons such as, but not limited to, neuron-specific enolase (NSE), platelet-derived growth factor (PDGF), platelet-derived growth factor B-chain (PDGF-β), the synapsin (Syn), the methyl-CpG binding protein 2 (MeCP2), Ca2+/calmodulin-dependent protein kinase II (CaMKII), metabotropic glutamate receptor 2 (mGluR2), NFL, NFH, np32, PPE, Enk and EAAT2 promoters. The transgene may comprise a tissue-specific expression element for astrocytes such as, but not limited to, the glial fibrillary acidic protein (GFAP) and EAAT2 promoters. The transgene may comprise tissue-specific expression elements for oligodendrocytes such as, but not limited to, the myelin basic protein (MBP) promoter.

In some embodiments, the promoter is less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. The promoter may provide expression of the therapeutic gene expression product for a period of time in targeted tissues such as, but not limited to, the heart. Expression of the therapeutic gene expression product may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years. Expression of the payload may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years or 10-15 years, or 15-20 years, or 20-25 years, or 25-30 years, or 30-35 years, or 35-40 years, or 40-45 years, or 45-50 years, or 50-55 years, or 55-60 years, or 60-65 years.

An AAV vector can comprise a genome of a helper virus. Helper virus proteins are required for the assembly of a recombinant AAV (rAAV), and packaging of a transgene containing a heterologous nucleic acid into the rAAV. The helper virus genes are adenovirus genes E4, E2a and VA, that when expressed in the cell, assist with AAV replication. In some embodiments, an AAV vector comprises E2. In some embodiments, an AAV vector comprises E4. In some embodiments, an AAV vector comprises VA. In some instances, the AAV vector comprises one of helper virus proteins, or any combination.

The target gene or gene expression product for use in a transgene can be selected from MYBPC3, MYH7, LMNA, SCN5A, DMD, TAZ, BAG3, DSC2, DSG2, DSP, PKP2, CSRP3, phosphatase inhibitor (1-1) protein, KCNH2, TRPM4, ATP2A2, CACNA1C, DMPK, EPG5, EVC, EVC2, FBN1, NF1, SOS1, NPR1, ERBB4, VIP, MYH7, TNNI3, relaxin and FXN.

An AAV vector can comprise a viral genome comprising a nucleic acid encoding the recombinant AAV (rAAV) capsid protein described herein. The viral genome can comprise a Replication (Rep) gene encoding a Rep protein, and Capsid (Cap) gene encoding an AAP protein in the first open reading frame (ORF1) or a Cap protein in the second open reading frame (ORF2). The Rep protein is selected from Rep78, Rep68, Rep52, and Rep40. In some instances, the Cap gene is modified encoding a modified AAV capsid protein described herein. A wild-type Cap gene encodes three proteins, VP1, VP2, and VP3. In some cases, VP1 is modified. In some cases, VP2 is modified. In some cases, VP3 is modified. In some cases, all three VP1-VP3 are modified. The AAV vector can comprise nucleic acids encoding wild-type Rep78, Rep68, Rep52, Rep40 and AAP proteins.

In some instances, the AAV9 VP1 gene provided in Table 4, is modified to include any one of variants listed in Tables 1, 2, 3, or FIG. 1. The AAV vector described herein may be used to produce a variant AAV capsid by the methods described herein.

TABLE 4
VP1 Capsid Protein Nucleic Acid Sequences

| Identifier | Sequence |
|---|---|
| AAV9 | ATGGCTGCCGATGGTTATCTTCCAGATTGGCTCGAGGACAACCTT |
| >AY530579.1 | AGTGAAGGAATTCGCGAGTGGTGGGCTTTGAAACCTGGAGCCCCT |
| Adeno- | CAACCCAAGGCAAATCAACAACATCAAGACAACGCTCGAGGTCT |
| associated | TGTGCTTCCGGGTTACAAATACCTTGGACCCGGCAACGGACTCGA |
| virus 9 | CAAGGGGGAGCCGGTCAACGCAGCAGACGCGGCGGCCCTCGAGC |
| isolate hu.14 | ACGACAAGGCCTACGACCAGCAGCTCAAGGCCGGAGACAACCCG |
| capsid | TACCTCAAGTACAACCACGCCGACGCCGAGTTCCAGGAGCGGCTC |
| protein VP1 | AAAGAAGATACGTCTTTTGGGGGCAACCTCGGGCGAGCAGTCTTC |
| (cap) gene, | CAGGCCAAAAAGAGGCTTCTTGAACCTCTTGGTCTGGTTGAGGAA |
| complete cds | GCGGCTAAGACGGCTCCTGGAAAGAAGAGGCCTGTAGAGCAGTC |
| | TCCTCAGGAACCGGACTCCTCCGCGGGTATTGGCAAATCGGGTGC |
| | ACAGCCCGCTAAAAAGAGACTCAATTTCGGTCAGACTGGCGACAC |
| | AGAGTCAGTCCCAGACCCTCAACCAATCGGAGAACCTCCCGCAGC |
| | CCCCTCAGGTGTGGGATCTCTTACAATGGCTTCAGGTGGTGGCGC |
| | ACCAGTGGCAGACAATAACGAAGGTGCCGATGGAGTGGGTAGTT |
| | CCTCGGGAAATTGGCATTGCGATTCCCAATGGCTGGGGGACAGAG |
| | TCATCACCACCAGCACCCGAACCTGGGCCCTGCCCACCTACAACA |
| | ATCACCTCTACAAGCAAATCTCCAACAGCACATCTGGAGGATCTT |
| | CAAATGACAACGCCTACTTCGGCTACAGCACCCCCTGGGGGTATT |
| | TTGACTTCAACAGATTCCACTGCCACTTCTCACCACGTGACTGGCA |
| | GCGACTCATCAACAACAACTGGGGATTCCGGCCTAAGCGACTCAA |
| | CTTCAAGCTCTTCAACATTCAGGTCAAAGAGGTTACGGACAACAA |
| | TGGAGTCAAGACCATCGCCAATAACCTTACCAGCACGGTCCAGGT |
| | CTTCACGGACTCAGACTATCAGCTCCCGTACGTGCTCGGGTCGGC |
| | TCACGAGGGCTGCCTCCCGCCGTTCCCAGCGGACGTTTTCATGATT |
| | CCTCAGTACGGGTATCTGACGCTTAATGATGGAAGCCAGGCCGTG |
| | GGTCGTTCGTCCTTTTACTGCCTGGAATATTTCCCGTCGCAAATGC |
| | TAAGAACGGGTAACAACTTCCAGTTCAGCTACGAGTTTGAGAACG |
| | TACCTTTCCATAGCAGCTACGCTCACAGCCAAAGCCTGGACCGAC |
| | TAATGAATCCACTCATCGACCAATACTTGTACTATCTCTCAAAGA |
| | CTATTAACGGTTCTGGACAGAATCAACAAACGCTAAAATTCAGTG |
| | TGGCCGGACCCAGCAACATGGCTGTCCAGGGAAGAAACTACATA |
| | CCTGGACCCAGCTACCGACAACAACGTGTCTCAACCACTGTGACT |
| | CAAAACAACAACAGCGAATTTGCTTGGCCTGGAGCTTCTTCTTGG |
| | GCTCTCAATGGACGTAATAGCTTGATGAATCCTGGACCTGCTATG |
| | GCCAGCCACAAAGAAGGAGAGGACCGTTTCTTTCCTTTGTCTGGA |
| | TCTTTAATTTTTGGCAAACAAGGAACTGGAAGAGACAACGTGGAT |
| | GCGGACAAAGTCATGATAACCAACGAAGAAGAAATTAAAACTAC |
| | TAACCCGGTAGCAACGGAGTCCTATGGACAAGTGGCCACAAACC |
| | ACCAGAGTGCCCAAGCACAGGCGCAGACCGGCTGGGTTCAAAAC |
| | CAAGGAATACTTCCGGGTATGGTTTGGCAGGACAGAGATGTGTAC |
| | CTGCAAGGACCCATTTGGGCCAAAATTCCTCACACGGACGGCAAC |
| | TTTCACCCTTCTCCGCTGATGGGAGGGTTTGGAATGAAGCACCCG |
| | CCTCCTCAGATCCTCATCAAAAACACACCTGTACCTGCGGATCCT |
| | CCAACGGCCTTCAACAAGGACAAGCTGAACTCTTTCATCACCCAG |
| | TATTCTACTGGCCAAGTCAGCGTGGAGATCGAGTGGGAGCTGCAG |
| | AAGGAAAACAGCAAGCGCTGGAACCCGGAGATCCAGTACACTTC |
| | CAACTATTACAAGTCTAATAATGTTGAATTTGCTGTTAATACTGAA |
| | GGTGTATATAGTGAACCCCGCCCCATTGGCACCAGATACCTGACT |
| | CGTAATCTGTAA |

Methods of Producing rAAVs

Disclosed herein are methods of producing the AAV capsids comprising the AAV capsid proteins and viral vector encoding a therapeutic nucleic acid. The AAV capsid proteins are produced by introducing into a cell (e.g., immortalized stem cell) a first vector containing a transgene cassette flanked by inverted terminal repeat (ITR) sequences from a parental AAV virus (the transgene cassette has a promoter sequence that drives transcription of a heterologous nucleic acid in the nucleus of the target cell), a second vector encoding the AAV genome with a AAV capsid protein (encoding the AAV Rep gene as well as the modified Cap gene for the variant being produced), and a third vector encoding helper virus proteins, required for assembly of the AAV capsid structure and packaging of the transgene in the modified AAV capsid structure. The assembled AAV capsid can be isolated and purified from the cell using suitable methods known in the art. Tables 1-2 provide DNA sequences for using in the methods described herein.

The transgenes contained in a recombinant AAV (rAAV) vector and encapsidated by the AAV capsid proteins of the present disclosure are also provided herein. The transgenes disclosed herein are delivered to a subject for a variety of purposes, such as to treat a disease or condition in the subject. The transgene can be gene editing components that modulate the activity or expression of a target gene or gene expression product. Alternatively, the transgene is a gene encoding a therapeutic gene expression product that is effective to modulate the activity or expression of itself, or another target gene or gene expression product.

Aspects disclosed herein provide methods of manufacturing rAAV virus or virus particles comprising: (a) introducing into a cell a nucleic acid comprising: (i) first vector containing a transgene cassette flanked by inverted terminal repeat (ITR) sequences from a parental AAV virus (the transgene cassette has a promoter sequence that drives transcription of a heterologous nucleic acid in the nucleus of the target cell); (ii) a second vector encoding the AAV genome with a AAV capsid protein of the present invention; and (iii) a vector encoding helper virus proteins, required for assembly of the AAV capsid structure and packaging of the transgene in the modified AAV capsid structure; (b) expressing in the cell the AAV capsid protein described herein; (c) assembling an AAV particle comprising the AAV capsid proteins disclosed herein; and (d) packaging the AAV particle. In some instances, the cell is mammalian. In some instances, the cell is immortalized. In some instances, the immortalized cell is an embryonic stem cell. In some instances, the embryonic stem cell is a human embryonic stem cell. In some instances, the human embryonic stem cell is a human embryonic kidney 293 (HEK-293) cell. In some instances, the 5′ ITR and the 3′ ITR are derived from an AAV2 serotype. In some instances, the 5′ ITR and the 3′ ITR are derived from an AAV5 serotype. In some instances, the 5′ ITR and the 3′ ITR are derived from an AAV9 serotype. In some instances, the first nucleic acid sequence and the second nucleic acid sequence are in trans. In some instances, the first nucleic acid sequence and the second nucleic acid sequence are in cis. In some instances, the first nucleic acid sequence, the second nucleic acid sequence and the third nucleic acid sequence, are in trans.

The Cap gene disclosed here comprises any one of sequences from Tables 1 or 2, or FIG. 1.

In some instances, the methods comprise packing the first nucleic acid sequence encoding the therapeutic gene expression product such that it becomes encapsidated by the modified AAV capsid protein. In some embodiments, the rAAV particles are isolated, concentrated, and purified using suitable viral purification methods, such as those described herein.

In some cases, rAAVs of the present disclosure are generated using the methods described in Challis, R. C. et al. Nat. Protoc. 14, 379 (2019). Briefly, triple transfection of HEK293T cells (ATCC) using polyethylenimine (PEI) is performed, viruses are collected after 120 hours from both cell lysates and media and purified over iodixanol. In a non-limiting example, the rAAVs are generated by triple transfection of precursor cells (e.g., HEK293T) cells using a standard transfection protocol (e.g., PEI). Viral particles are harvested from the media after a period of time (e.g., 72 h post transfection) and from the cells and media at a later point in time (e.g., 120 h post transfection). Virus present in the media is concentrated by precipitation with 8% polyethylene glycol (PEG) and 500 mM sodium chloride and the precipitated virus is added to the lysates prepared from the collected cells. The viruses are purified over iodixanol (Optiprep, Sigma) step gradients (15%, 25%, 40% and 60%). Viruses are concentrated and formulated in PBS. Virus titers are determined by measuring the number of DNaseI-resistant vector genome copies (VGs) using qPCR and the linearized genome plasmid as a control.

The cell can be selected from a human, a primate, a murine, a feline, a canine, a porcine, an ovine, a bovine, an equine, an epine, a caprine and a lupine host cell. In some instances, the cell is a progenitor or precursor cell, such as a stem cell. In some instances, the stem cell is a mesenchymal cell, embryonic stem cell, induced pluripotent stem cell (iPSC), fibroblast or other tissue specific stem cell. The cell can be immortalized. In some cases, the immortalized cell is a HEK293cell. In some instances, the cell is a differentiated cell. Based on the disclosure provided, it is expected that this system can be used in conjunction with any transgenic line expressing a recombinase in the target cell type of interest to develop AAV capsids that more efficiently transduce that target cell population.

### Methods of Treatment

Disclosed herein are methods of treating a disease or condition, or a symptom of the disease or condition, in a subject, comprising administrating of therapeutically effective amount of one or more compositions (e.g., rAAV particle, AAV vector, pharmaceutical composition) disclosed herein to the subject. In some embodiments, the composition is a rAAV capsid protein described herein. In some embodiments, the composition is an isolated and purified rAAV capsid protein described herein. In some embodiments, the rAAV particle encapsidates an AAV vector comprising a transgene (e.g., therapeutic nucleic acid). In some embodiments, the composition is a rAAV capsid protein described herein conjugated with a therapeutic agent disclosed herein. In some embodiments, the composition is a pharmaceutical composition comprising the rAAV particle and a pharmaceutically acceptable carrier. In some embodiments, the one or more compositions are administered to the subject alone (e.g., stand-alone therapy). In some embodiments, the composition is a first-line therapy for the disease or condition. In some embodiments, the composition is a second-line, third-line, or fourth-line therapy, for the disease or condition.

Recombinant adeno-associated virus (rAAV) mediated gene delivery leverages the AAV mechanism of viral transduction for nuclear expression of an episomal heterologous nucleic acid (e.g., a transgene, therapeutic nucleic acid). Upon delivery to a host in vivo environment, a rAAV will (1) bind or attach to cellular surface receptors on the target cell, (2) endocytose, (3) traffic to the nucleus, (4) uncoat the virus to release the encapsidated heterologous nucleic acid, (5) convert of the heterologous nucleic acid from single-stranded to double-stranded DNA as a template for transcription in the nucleus, and (6) transcribe of the episomal heterologous nucleic acid in the nucleus of the host cell (“transduction”). rAAVs engineered to have an increased transduction enrichment (transcription of the episomal heterologous nucleic acid in the host cell) are desirable for gene therapy applications.

Aspects disclosed herein provide methods of treating a disease or condition in a subject, the method comprising administering to the subject a therapeutically effective amount of the rAAV of the present disclosure, or the pharmaceutical formulation of the present disclosure, wherein the gene product is a therapeutic gene product. In some embodiments, the administering is by intracranial, intraventricular, intracerebroventricular, intravenous, intraarterial, intranasal, intrathecal, intracisternae magna, or subcutaneous.

Provided here, are methods of treating a disease or a condition associated with an aberrant expression or activity of a target gene or gene expression product thereof, the method comprising modulating the expression or the activity of a target gene or gene expression product in a subject by administering a rAAV encapsidating a heterologous nucleic acid of the present disclosure. In some instances, the expression or the activity of the target gene or gene expression product is decreased, relative to that in a normal (non-diseased) individual; and administering the rAAV to the subject is sufficient to increase the expression of the activity of the target gene or gene expression product. In some instances, the expression or the activity of the gene or gene expression product is increased, relative to that in a normal individual; and administering the rAAV to the subject is sufficient to decrease the expression or the activity of the target gene or gene expression product. In a non-limiting example, a subject diagnosed with Alzheimer's disease, which is caused, in some cases, by a gain-of-function of a Presenilin I and/or Presenilin 2 (encoded by the gene PSEN1 and PSEN2, respectively) is administered a rAAV disclosed herein encapsidating a therapeutic nucleic acid that is a silencing RNA (siRNA), or other RNAi with a loss-of-function effect on PSEN1 mRNA.

Also provided are methods of preventing a disease or condition disclosed herein in a subject comprising administering to the subject a therapeutically effective amount of an rAAV vector comprising a nucleic acid sequence encoding a therapeutic gene expression product described herein. The rAAV vector may be encapsidated in the modified capsid protein or rAAV viral particle described herein. In some instances, the therapeutic gene expression product is effective to modulate the activity or expression of a target gene or gene expression product.

Disclosed herein are methods of treating a disease or condition in a subject by administering a composition comprising a rAAV disclosed herein. An advantage of the rAAVs disclosed herein, is that the rAAV may be used to treat virtually any disease or condition that would benefit from a transgene therapy, including but not limited to Friedreich's Ataxia, Lysosomal storage disorders including Pompe Disease and Danon Disease, Chronic heart failure, congestive heart failure, Cardiomyopathies including Hypertrophic myopathies, Dilated myopathies and Arrhythmogenic myopathies, and arrhythmia.

In some cases, the disease or condition is localized to a particular in vivo environment in the subject, e.g., the heart. The compositions of the present disclosure are particularly useful for the treatment of the diseases or conditions described herein because they specifically or more efficiently target the in vivo environment and deliver a therapeutic nucleic acid engineered to modulate the activity or the expression of a target gene expression product involved with the pathogenesis or pathology of the disease or condition.

Provided herein are methods of treating a disease or a condition, or a symptom of the disease or condition, in a subject, comprising: (a) diagnosing a subject with a disease or a condition affecting a target in vivo environment; and (b) treating the disease or the condition by administering to the subject a therapeutically effective amount of a composition disclosed herein (e.g., rAAV particle, AAV vector, pharmaceutical composition), wherein the composition is engineered with an increased specificity for the target in vivo environment.

Disclosed herein are methods of treating a disease or a condition, or a symptom of the disease or the condition, afflicting a target in a subject comprising: (a) administering to the subject a composition (e.g., rAAV particle, AAV vector, pharmaceutical composition); and (b) expressing the therapeutic nucleic acid into a target in vivo environment in the subject with an increased transduction enrichment.

In some embodiments, methods further comprise reducing or ablating delivery of the heterologous nucleic acid in an off-target in vivo environment, such as the liver. In some embodiments, delivery is characterized by an increase in enrichment of transduction (e.g., of the heterologous nucleic acid) in the heart.

In some embodiments, methods of treating a disease or condition affecting the heart comprise administering a rAAV particle to a heart in a subject, the rAAV particle comprising an rAAV capsid protein comprising an insertion of about, five, six, or seven amino acids of an amino acid sequence provided in Tables 1-2, FIG. 1 and Formulas I-VII, at an amino acid position 588-589 in a parental AAV capsid protein. In some embodiments, methods of treating a disease or condition affecting the heart comprise administering a rAAV particle to a heart in a subject, the rAAV particle comprising an rAAV capsid protein comprising an insertion of about, five, six, or seven amino acids of an amino acid sequence as well as one or more substitution at amino acid found at amino acid positions 587-590 [AQAQ] such as provided in Tables 1-2, FIG. 1 and Formulas I-VII. In some embodiments, the parental AAV capsid protein is AAV9 capsid protein (for e.g., provided in SEQ ID NO: 1.

Also provided are methods of modulating a target gene expression product, the methods comprising administering to a subject in need thereof a composition (e.g., rAAV particle, AAV vector, pharmaceutical composition) disclosed herein. For example, methods provided herein comprise administering to a subject a rAAV with a rAAV capsid protein encapsidating a viral vector comprising a heterologous nucleic acid that modulates the expression or the activity of the target gene expression product.

The term “normal individual” refers to an individual that is not afflicted with the disease or the condition characterized by the variation in expression or activity of the gene or gene expression product thereof.

In some examples, a method is useful to treat a degenerative muscle disease or condition (e.g., familial cardiomyopathy, dilated cardiomyopathy, hypertrophic cardiomyopathy, restrictive cardiomyopathy, or coronary artery disease with resultant ischemic cardiomyopathy). In some examples, a subject method is useful to treat individuals having a cardiac or cardiovascular disease or disorder, for example, cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism. In some examples, the subject is suffering from or at risk for cardiomyopathy.

In some embodiments, the disease or condition of the heart is selected from Friedreich's Ataxia, Lysosomal storage disorders including Pompe Disease and Danon Disease, Chronic heart failure, congestive heart failure, Cardiomyopathies including Hypertrophic myopathies, Dilated myopathies and Arrhythmogenic myopathies, and arrhythmia.

In some embodiments, the pharmaceutical formulation comprises a therapeutic nucleic acid encoding a therapeutic gene expression product. In some instances, the therapeutic gene expression product is effective to modulate an activity or an expression of a target gene or gene expression product selected from MYBPC3, MYH7, LMNA, SCN5A, DMD, TAZ, BAG3, DSC2, DSG2, DSP, PKP2, CSRP3, phosphatase inhibitor (1-1) protein, KCNH2, TRPM4, ATP2A2, CACNA1C, DMPK, EPG5, EVC, EVC2, FBN1, NF1, SOS1, NPR1, ERBB4, VIP, MYH7, TNNI3, relaxin and FXN.

In some instances, the therapeutic gene expression product comprises gene editing components. In some instances, the gene editing components are selected from an artificial site-specific RNA endonuclease (ASRE), a zinc finger endonuclease (ZFN), a transcription factor like effector nuclease (TALEN), a clustered regularly interspaced short palindromic repeats (CRISPR)/Cas enzyme, and a CRISPR)/Cas guide RNA.

In some instances, the expression of a gene or expression or activity of a gene expression product is inhibited by the administration of the composition to the subject. In some instances, the expression of a gene or the expression or the activity of a gene expression product is enhanced by the administration of the composition to the subject.

### Formulations, Dosages, and Routes of Administration

Disclosed herein are methods comprising delivering a rAAV particle encapsidating a heterologous nucleic acid to the heart in a subject, the rAAV particle comprising (i) an increased transduction of the heterologous nucleic acid in the heart, wherein the rAAV particle has an rAAV capsid protein comprising an insertion of five, six, or seven amino acids of an amino acid sequence provided in Tables 1-2, FIG. 1 and Formulas I-VII, at an amino acid position 588-589 in a parental AAV capsid protein as well as one or more substitution at amino acid found at amino acid positions 587-590 [AQAQ] such as provided in Tables 1-2, FIG. 1 and Formulas I-VII.

In general, methods disclosed herein comprise administering a therapeutic rAAV composition by systemic administration. In some instances, methods comprise administering a therapeutic rAAV composition by intravenous (“i.v.”) administration. One may administer therapeutic rAAV compositions by additional routes, such as subcutaneous injection, intramuscular injection, intradermal injection, transdermal injection, percutaneous administration, intranasal administration, intralymphatic injection, rectal administration intragastric administration, intraocular administration, intracerebroventricular administration, intrathecally, intracisternal, or any other suitable parenteral administration. Routes, dosage, time points, and duration of administrating therapeutics may be adjusted. In some embodiments, administration of therapeutics is prior to, or after, onset of either, or both, acute and chronic symptoms of the disease or condition. Other routes of delivery to the heart include, but are not limited to intracranial administration, lateral cerebroventricular administration, and endovascular administration.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration may be changed, or an additional agent may be administered to the subject, along with the therapeutic rAAV composition. In some embodiments, as a patient is started on a regimen of a therapeutic rAAV composition, the patient is also weaned off (e.g., step-wise decrease in dose) a second treatment regimen.

In some cases, a dose of the pharmaceutical composition may comprise a concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases, the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, or 2×10¹⁷. In some cases, the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, or 3×10¹⁷. In some cases, the concentration of the infectious particles is 4×10⁷, 4×10⁸, 4×10⁹, 4×10¹⁰, 4×10¹¹, 4×10¹², 4×10¹³, 4×10¹⁴, 4×10¹⁵, 4×10¹⁶, or 4×10¹⁷. In some cases, the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, or 5×10¹⁷. In some cases, the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, or 6×10¹⁷. In some cases, the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10¹⁶, or 7×10¹⁷. In some cases, the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, or 8×10¹⁷. In some cases, the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10¹⁶, or 9×10¹⁷.

Disclosed herein, in some embodiments are formulations of pharmaceutically-acceptable excipients and carrier solutions suitable for delivery of the rAAV compositions described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, the pharmaceutical forms of the rAAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

In some cases, for administration of an injectable aqueous solution, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the rAAV compositions disclosed herein, which are prepared by incorporating the rAAV compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions may be advantageous for systemic administration, for example by intravenous or intrathecal administration.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of rAAV compositions and time of administration of such compositions will be within the purview of the skilled artisan having benefit of the present teachings. It is likely, however, that the administration of therapeutically-effective amounts of the disclosed compositions may be achieved by a single administration, example, a single injection of sufficient numbers of infectious particles to provide therapeutic benefit to the patient undergoing such treatment. This is made possible, at least in part, by the fact that certain target cells (e.g., neurons) do not divide, obviating the need for multiple or chronic dosing.

In certain embodiments, the data obtained from cell culture assays and animal studies are used in formulating the therapeutically effective daily dosage range and/or the therapeutically effective unit dosage amount for use in mammals, including humans. In certain embodiments, the dosage range and/or the unit dosage amount varies within this range depending upon the dosage form employed and the route of administration utilized.

### Combination Therapies

A therapeutic rAAV may be used alone or in combination with an additional therapeutic agent (together, “therapeutic agents”). In some cases, a therapeutic rAAV as used herein is administered alone. The therapeutic agent may be administered together or sequentially in a combination therapy. The combination therapy may be administered within the same day, or may be administered one or more days, weeks, months, or years apart.

The additional therapeutic agent can comprise a small molecule. The additional therapeutic agent can comprise an antibody, or antigen-binding fragment. The additional therapeutic agent can include lipid nanoparticle-based therapies, anti-sense oligonucleotide therapies, as well as other viral therapies.

The additional therapeutic agent can comprise a cell-based therapy. Exemplary cell-based therapies include without limitation immune effector cell therapy, chimeric antigen receptor T-cell (CAR-T) therapy, natural killer cell therapy and chimeric antigen receptor natural killer (NK) cell therapy. Either NK cells, or CAR-NK cells, or a combination of both NK cells and CAR-NK cells can be used in combination with the methods disclosed herein. In some embodiments, the NK cells and CAR-NK cells are derived from human induced pluripotent stem cells (iPSC), umbilical cord blood, or a cell line. The NK cells and CAR-NK cells can comprise a cytokine receptor and a suicide gene. The cell-based therapy can comprise a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject.

### Kits

Disclosed herein are kits comprising compositions disclosed herein. Also disclosed herein are kits for the treatment or prevention of a disease or conditions of the heart. In some instances, the disease or condition is cancer, a pathogen infection, pulmonary disease or condition, neurological disease, muscular disease, or an immune disorder, such as those described herein.

In one embodiment, a kit can include a therapeutic or prophylactic composition containing an effective amount of a composition of a rAAV particle encapsidating a recombinant AAV vector encoding a therapeutic nucleic acid (e.g., therapeutic nucleic acid) and a recombinant AAV (rAAV) capsid protein of the present disclosure. In another embodiment, a kit can include a therapeutic or prophylactic composition containing an effective amount of cells modified by the rAAV described herein (“modified cell”), in unit dosage form that express therapeutic nucleic acid. In some embodiments, a kit comprises a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some instances, the kit further comprises a cell. In some instances, the cell is mammalian. In some instances, the cell is immortalized. In some instances, the immortalized cell is an embryonic stem cell. In some instances, the embryonic stem cell is a human embryonic stem cell. In some instances, the human embryonic stem cell is a human embryonic kidney 293 (HEK-293) cell. In some instances, the kit further comprises an AAV vector comprising a heterologous nucleic acid encoding a therapeutic gene expression product. In some instances, the AAV vector is an episome.

In some cases, rAAV are provided together with instructions for administering the rAAV to a subject having or at risk of developing the disease or condition (e.g., disease of the heart). Instructions can generally include information about the use of the composition for the treatment or prevention of the disease or condition.

In some cases, the instructions include at least one of the following: description of the therapeutic rAAV composition; dosage schedule and administration for treatment or prevention of the disease or condition disclosed herein; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. In some cases, instructions provide procedures for administering the rAAV to the subject alone. In some instances, the instructions provide that the rAAV is formulated for systemic delivery.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating skin disorders like acne, eczema, psoriasis, and rosacea.

The terms “homologous,” “homology,” or “percent homology” are used herein to generally mean an amino acid sequence or a nucleic acid sequence having the same, or similar sequence to a reference sequence. Percent homology of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “subject” is any organism. In some instances, the organism is a mammal. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In one embodiment, a “non-human primate” is a mammal, for example a monkey. In some instances, the subject is a patient, which as used herein, may refer to a subject diagnosed with a particular disease or disorder.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence.

The term “adeno-associated virus,” or “AAV” as used herein refers to the adeno-associated virus or derivatives thereof. Non-limited examples of AAV's include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV 11), AAV type 12 (AAV12), avian AAV, bovine AAV, canine AAV, equine AAV, primate AAV, non-primate AAV, and ovine AAV. In some instances, the AAV is described as a “Primate AAV.” which refers to AAV that infect primates. Likewise an AAV may infect bovine animals (e.g., “bovine AAV”, and the like). In some instances, the AAV is wildtype, or naturally occurring. In some instances, the AAV is recombinant.

The term “AAV capsid” as used herein refers to a capsid protein or peptide of an adeno-associated virus. In some instances, the AAV capsid protein is configured to encapsidate genetic information (e.g., a transgene, therapeutic nucleic acid, viral genome). In some instances, the AAV capsid of the instant disclosure is a modified AAV capsid, relative to a corresponding parental AAV capsid protein.

The term “tropism” as used herein refers to a quality or characteristic of the AAV capsid that may include specificity for, and/or an increase or a decrease in enrichment of, expressing the encapsidated genetic information into an in vivo environment, relative to a second in vivo environment. An in vivo environment, in some instances, is a cell-type. An in vivo environment, in some instances, is an organ or organ system.

The term “AAV vector” as used herein refers to nucleic acid polymer encoding genetic information related to the virus. The AAV vector may be a recombinant AAV vector (rAAV), which refers to an AAV vector generated using recombinatorial genetics methods. In some instances, the rAAV vector comprises at least one heterologous polynucleotide (e.g. a polynucleotide other than a wild-type or naturally occurring AAV genome such as a transgene).

The term “AAV particle” as used herein refers to an AAV virus, virion, AAV capsid protein or component thereof. In some cases, the AAV particle is modified relative to a parental AAV particle.

The term “gene product” of “gene expression product” refers to an expression product of a polynucleotide sequence such as, for e.g., a polypeptide, peptide, protein or RNA, including interfering RNA (e.g., siRNA, miRNA, shRNA) and messenger RNA (mRNA).

The term “heterologous” as used herein refers to a genetic element (e.g., coding region) or gene expression product (e.g., RNA, protein) that is derived from a genotypically distinct entity from that of the rest of the entity to which it is being compared.

The term “endogenous” as used herein refers to a genetic element (e.g., coding region) or gene expression product (e.g., RNA, protein) that is naturally occurring in or associated with an organism or a particular cell within the organism.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, FL, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, systemic administration.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. Alternatively, a sample can be obtained through primary patient derived cell lines, or archived patient samples in the form of preserved samples, or fresh frozen samples.

The term “in vivo” is used to describe an event that takes place in a subject's body.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The term “heart” or “cardiovascular system” means a tissue selected from the heart, veins and aortas.

The term “systemic delivery” is defined as a route of administration of medication or other substance into a circulatory system so that the entire body is affected. Administration can take place via enteral administration (absorption of the drug through the gastrointestinal tract) or parenteral administration (generally injection, infusion, or implantation), “Circulatory system” includes a blood circulatory system Examples of systemic administration include intraarterial, intravenous or intrathecal injection. The terms “systemic administration” and “systemic delivery” are used interchangeably.

The section headings used herein are for organizational purposes only and are not to be construed as limiting the subject matter described.

### EXAMPLES

### Example 1

### Method of Identifying the Modified Capsid Proteins in Cynomolgus Macaques

Of primary concern for the therapeutic applicability of engineered adeno-associated viruses (AAVs) is how well their transduction profiles translate to human application. While previous engineering efforts have focused on in vitro or in vivo rodent screening platforms due to the ease and flexibility of their use, screening efforts directly in non-human primates (NHPs) are much more likely to identify viruses that translate. We chose cynomolgus macaques, an old world NHP, for our engineering efforts. We focused our engineering efforts on a region of the AAV9 capsid surface located at amino acid position 588, one of the most exposed loops on the capsid surface that is a variable region between natural AAV serotypes and has a role in receptor binding. We created a library of viral capsid by performing a random 7 amino acid insertion at this site within AAV9, and analyzed for novel tropism toward the NHP heart.

Plasmids. The first-round viral DNA library was generated by amplification of a section of the AAV9 capsid genome between amino acids 450-599 using NNK degenerate primers (Integrated DNA Technologies, Inc., IDT) to insert seven random amino acids between amino acids 588 and 589 with all possible variations. The resulting library inserts were then introduced into the rAAV-ACap-in-rev-RNA plasmid via Gibson assembly as previously described. The resulting capsid DNA library, rAAV-Cap-Cag-GFP11, contained a diversity of ˜1.28 billion variants at the amino acid level. The second round viral DNA library was generated similarly to the first round, but instead of NNK degenerate primers inserted at the 588, a synthesized oligo pool (Twist Biosicence) was used to generate only selected variants in a UBC-Cap-DNA and CAG-Cap-DNA construct with CAP. This second-round DNA library contained a diversity of 20,000 variants at the amino acid level with 3 codon replicates for each variant. The AAV2/9 REP-AAP-ACAP plasmid transfected into HEK293T cells to provide the Rep gene for library viral production prevents production of a wild-type AAV9 capsid during viral library production after a plausible recombination event between this plasmid co-transfected with the library plasmids at each stage containing the library inserts.

Viral production. Recombinant AAVs were generated according to established protocols. Briefly, immortalized HEK293T cells (ATCC) were quadruple transfected with four vectors using polyethylenimine (PEI). The first vector was the rAAV-Cap-in-cis-Lox library flanked by inverted terminal repeat (ITR) sequences from a parental AAV virus. The second vector was the AAV2/9 REP-AAP-ACAP plasmid. The third vector contains nucleic acids encoding helper virus proteins needed for viral assembly and packaging of the heterologous nucleic acid into the modified capsid structure. The fourth is a pUC-18 plasmid included to achieve the right PEI/DNA ratio for optimal transfection enrichment. Only 10 ng of rAAV-Cap-in-cis-Lox library DNA was transfected (per 150 mm plate) to decrease the likelihood of multiple library DNAs entering the same cell. Viral particles are harvested from the cells and media after 60 h post transfection. Virus present in the media is concentrated by precipitation with 8% polyethylene glycol and 500 mM sodium chloride and the precipitated virus is added to the lysates prepared from the collected cells. The viruses are purified over iodixanol (Optiprep, Sigma) step gradients (15%, 25%, 40%, and 60%). Viruses are concentrated and formulated in PBS. Virus titers are determined by measuring the number of DNaseI-resistant vector genome copies (VGs) using qPCR and the linearized genome plasmid as a control.

Animals. Cynomolgus macaque procedures were approved by the IACUC committee of Envol Biomedical. Cynomolgus macaque were born and raised in Envol Biomedical colonies and housed in family groups under standard conditions. They were fed ad libitum and received enrichment as part of the primate enrichment program for NHPs at Envol Biomedical. For AAV infusions, animals were screened for endogenous neutralizing antibodies (Nab). None of the animals that were screened showed any detectible blocking reaction at 1:10 dilution of serum. They were then group housed for several days and acclimated to a new room before injections. Four total juvenile monkeys were used for the screenings, two for the first round library and two for the second round library. Animals were restrained and the test article was administered via intravenous infusion for 10 minutes. Activity and behavior were monitored daily over the course of in-life.

DNA/RNA recovery and sequencing. Round 1 and round 2 viral libraries were injected into Cynomolgus Macaques at a dose of 2.5×10¹³ vg/kg animal and rAAV genomes were recovered two weeks post injection. Animals were euthanized and recovered, snap frozen, and placed into long-term storage at −80° C. as well as other peripheral tissues such as heart, spleen, adrenal, kidney, and quad. For round 1, the brain was separated into eleven brain regions, 100 mg each, and for round 2, thirteen brain regions. 100 mg of each heart, and liver was homogenized in buffer using the MagMAX DNA ULTRA (A25597) and a Bead Ruptor 96 (OMNI, INC) and viral DNA was isolated according to the manufacturers recommended protocol. Recovered viral DNA was treated with RNase and purified with a Zymo DNA Clean and Concentrator kit (D4033). Viral genomes were enriched by 25 cycles of PCR amplification with primers flanking the 588-589 insertion site in the capsid genome using 50% of the total extracted viral DNA as a template. After Zymo DNA purification, samples were diluted 1:10 to 1:1000 depending on tissue type and each dilution further amplified around the library variable region with 10 cycles of PCR. Subsequently, samples were further amplified using custom primers with Illumina Indices for 10 more cycles. The amplification products were run on a 2% low-melting point agarose gel (ThermoFisher Scientific, 16520050) for better separation and recovery of the 210 bp band.

For the second round library only, packaged viral library DNA was isolated from the injected viral library by digestion of the viral capsid and purification of the contained ssDNA. These viral genomes were amplified by two PCR amplification steps, like the viral DNA extracted from tissue, to add adapters and indices for Illumina next-generation sequencing, and purified after gel electrophoresis. This viral library DNA, along with the viral DNA extracted from tissue, was sent for deep sequencing using an Illumina NextSeq 2000 system.

NGS data alignment and processing. Raw fastq files from NGS runs were processed with custom-built scripts (Capsida CapSeq Tools). For the first round library, the pipeline to process these datasets involved filtering to remove low-quality reads, utilizing a quality score for each sequence, and eliminating bias from PCR-induced mutations or high GC-content. The filtered dataset was then aligned by a perfect string match algorithm and trimmed to improve the alignment quality. Read counts for each sequence were pulled out and displayed by tissue, at which point all sequences found in the heart were compiled for formation of the second round library.

For the second round library read counts by tissue were similarly tabulated. Then, a read count of 1 was added to each sequence to remove 0 values, all heart regions for each sequence were summed together, and the read sequences for each codon replicate of a given 7-mer amino acid sequence were summed together to give a single value for each peptide insertion. Finally, the data was log 10 counts per million (Cpm) normalized. Enrichment values were calculated with normalized cpm of heart vs cpm of virus and converted to log 10.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

What is claimed is:

1. An AAV capsid protein comprising a sequence as provided in Tables 1-2 or FIG. 1.

2. An AAV capsid comprising an insertion sequence of Formula I
X¹-G-G-X²-X³-X⁴-X⁵ (I)
wherein X¹ is an amino acid selected from G, I, K, L and V; X² is an amino acid selected from E, G, H, L, N, S and Q; X³ is an amino acid selected from A, F, I, L, P, T and Y; X⁴ is an amino acid selected from F, L, S and Y; and X⁵ is an amino acid selected from E, H, I, K, P, S and T.

3. The AAV capsid protein of claim 2, wherein X¹ is I, K or L.

4. The AAV capsid protein of claim 2, wherein X¹ is K.

5. The AAV capsid protein of claim 2, wherein X¹ is I.

6. The AAV capsid protein of claim 2, wherein X² is L, H or Q.

7. The AAV capsid protein of claim 2, wherein X² is L.

8. The AAV capsid protein of claim 2, wherein X² is H.

9. The AAV capsid protein of claim 2, wherein X³ is I, P or Y.

10. The AAV capsid protein of claim 2, wherein X⁴ is an F or L.

11. The AAV capsid protein of claim 2, wherein X⁵ is an I or H.

12. The AAV capsid protein of claim 2, wherein the sequence is selected from KGGLPFI, IGGHYLH, LGGQILI, LGGGFSE, IGGNLLK, KGGETSS, VGGSTYP, and GGGLAST.

13. An AAV capsid protein comprising an insertion sequence of Formula II
R-S-X⁶-X⁷-X⁸-X⁹-X¹⁰ (II)
wherein X⁶ is an amino acid selected from E, G, I, N, Q and T; X⁷ is an amino acid selected from L, H, Q, S, and T; X⁸ is an amino acid selected from D, E, H, P, and S; X⁹ is an amino acid selected from A, F, L, R, S and T; and X¹⁰ is an amino acid selected from E, I, M, S and V.

14. The AAV capsid protein of claim 13, wherein X¹⁰ is I, V or M.

15. The AAV capsid protein of claim 13, wherein X¹⁰ is M.

16. The AAV capsid protein of claim 13, wherein X⁹ is F or L.

17. The AAV capsid protein of claim 13, wherein X⁸ is P or H.

18. The AAV capsid protein of claim 13, wherein X⁷ is H or L.

19. The AAV capsid protein of claim 13, wherein X⁶ is E or G.

20. The AAV capsid protein of claim 13, wherein the insertion sequence is selected from RSGHPFV, RSGLHLM, RSELHFI, RSQTSTE, RSISDRV, RSTTDAS, and RSNQESV.

21. An AAV capsid protein comprising an insertion sequence of Formula III
R-X¹¹-G-X¹²-X¹³-X¹⁴-X¹⁵ (III)
wherein X¹¹ is an amino acid selected from A, D, E, I, N, V and S; X¹² is an amino acid selected from E, H, L, M, S and V; X¹³ is an amino acid selected from F, H, L, P, V and Y; X¹⁴ is an amino acid selected from F, L and T; and X¹⁵ is an amino acid selected from D, H, K, L, M, Q, S and V.

22. The AAV capsid protein of claim 21, wherein X¹ is an amino acid selected from A, D, N and S.

23. The AAV capsid protein of claim 21, wherein X² is an amino acid selected from E, H, L, and V.

24. The AAV capsid protein of claim 21, wherein X¹³ is an amino acid selected from F, H, P, and Y.

25. The AAV capsid protein of claim 21, wherein X⁴ is F or L.

26. The AAV capsid protein of claim 21, wherein X⁵ is an amino acid selected from K, L, M and V.

27. The AAV capsid protein of claim 21, wherein the insertion sequence is selected from RSGHPFV, RNGEHFK, RDGHYLL, RNGEFFH, REGMPFL, RAGVHFV, RSGLHLM, RIGVHTS, RIGSVTQ, and RVGSLTD.

28. An AAV capsid protein comprising an insertion sequence of Formula IV
X¹⁶-X¹⁷-G-H-X¹⁸-X¹⁹-X²⁰ (IV)
wherein X¹⁶ is an amino acid selected from I, R, and L; X¹⁷ is an amino acid selected from D, G, Q, and S; X¹⁸ is an amino acid selected from I, P and Y; X¹⁹ is F or L; and X²⁰ is an amino acid selected from H, L and V.

29. The AAV capsid protein of claim 28, wherein X¹⁶ is L or R.

30. The AAV capsid protein of claim 28, wherein X⁷ is an amino acid selected from P, Q and S.

31. The AAV capsid protein of claim 28, wherein X⁹ is F or L.

32. The AAV capsid protein of claim 28, wherein X²⁰ is L.

33. The AAV capsid protein of claim 28, wherein X²⁰ is V.

34. The AAV capsid protein of claim 28, wherein X¹⁸ is I or Y.

35. The AAV capsid protein of claim 28, wherein the insertion sequence as described in FIG. 1, is selected from RSGHPFV, RDGHYLL, LQGHILL, and IGGHYLH.

36. An AAV capsid protein comprising an AAV capsid protein comprising an insertion sequence of Formula V
R-X²¹-X²²-X²³-H-F-X²⁴ (V)
Wherein X²¹ is an amino acid selected from A, N and S; X²² is an amino acid selected from D, E and G; X²³ is an amino acid selected from E, L and V; and X²⁴ is an amino acid selected from I, K, Q and V.

37. The AAV capsid protein of claim 36, wherein X²¹ is A.

38. The AAV capsid protein of claim 36, wherein X²² is D.

39. The AAV capsid protein of claim 36, wherein X²³ is E.

40. The AAV capsid protein of claim 36, wherein X²³ is L.

41. The AAV capsid protein of claim 36, wherein X²⁴ is Q.

42. The AAV capsid protein of claim 36, wherein the insertion sequence is selected from RADLHFQ, RAGVHFV, RNGEHFK, and RSELHFI.

43. An AAV capsid protein comprising an AAV capsid protein comprising an insertion sequence of Formula VI
X²⁵-X²⁶-X²⁷-X²⁸-X²⁹-L-L (VI)
Wherein X²⁵ is an amino acid selected from A, L, R, and Y; X²⁶ is an amino acid selected from Q, E, D, T, K and F; X²⁷ is an amino acid selected from G, Q, and A; X²⁸ is an amino acid selected from H, G, T, V and D; and X²⁹ is an amino acid selected from I, S, T and Y.

44. The AAV capsid protein of claim 43, wherein X²⁵ is L or R.

45. The AAV capsid protein of claim 43, wherein X²⁶ is an amino acid selected from Q, E, and D.

46. The AAV capsid protein of claim 43, wherein X²⁷ is G, or Q.

47. The AAV capsid protein of claim 43, wherein X²⁸ is H, or G.

48. The AAV capsid protein of claim 43, wherein X²⁹ is I, or Y.

49. The AAV capsid protein of claim 43, wherein the insertion sequence is selected from LQGHILL, REQGYLL, RDGHYLL, YTGTSLL, AKAVTLL, and AFGDSLL.

50. An AAV capsid protein comprising an insertion sequence of Formula VII
X³⁰-X³¹-X³²-X³³-X³⁴-S-S (VII)
wherein X³⁰ is an amino acid selected from A, E, F, H, I, K, L, M, N, P, Q, S, T, V and Y; X³¹ is an amino acid selected from F, G, H, I, K, L, M, P, Q, R, S, T, and V; X³² is an amino acid selected from A, G, H, L, M, P, Q, S and V; X³³ is an amino acid selected from E, G, H, I, P, Q, R, S, T and V; and X³⁴ is an amino acid selected from D, G, I, L, M, N, R, S, T and V.

51. The AAV capsid protein of claim 50, wherein X³⁰ is V, Q or H.

52. The AAV capsid protein of claim 50, wherein X³¹ is F, L or M.

53. The AAV capsid protein of claim 50, wherein X³² is S, A or Q.

54. The AAV capsid protein of claim 50, wherein X³³ is G, H or S.

55. The AAV capsid protein of claim 50, wherein X³⁴ is G, V or M.

56. The AAV capsid protein of claim 50, wherein the sequence is selected from VFSGVSS, QLQHGSS, HMASMSS, SSLTLSS, SVPSSSS, ITPSVSS, MTVQVSS, SHLSVSS, YPHQTSS, PQGGSSS, FRSSLSS, ISVSISS, ETQRGSS, IHPPSSS, TSAITSS, FKSQSSS, ASHSTSS, AVMRDSS, KGGETSS, VVSVTSS, LISIRSS, NSLGLSS, and HLAQNSS.

57. An AAV capsid protein comprising an insertion sequence of Formula VIII
D-L-X³⁵-X³⁶-X³⁷-X³⁸-X³⁹ (VIII)
wherein X³⁵ is an amino acid selected from G, H, Q, S and T; X³⁶ is an amino acid selected from K, N, P, R and S; X³⁷ is an amino acid selected from H, L, R and S; X³⁸ is an amino acid selected from D, K, R, S, T and V; and X³⁹ is an amino acid selected from A, D, E, L and V.

58. The AAV capsid protein of claim 57, wherein X³⁵ is H or S.

59. The AAV capsid protein of claim 57, wherein X³⁶ is K, or R.

60. The AAV capsid protein of claim 57, wherein X³⁷ is L, or S.

61. The AAV capsid protein of claim 57, wherein X³⁸ is S, or V.

62. The AAV capsid protein of claim 57, wherein X³⁹ is A, or L.

63. The AAV capsid protein of claim 57, wherein the sequence is selected from DLSRLSL, DLHKSVA, DLGNLKV, DLTPHKD, DLQRSDL, and DLGSRRE.

64. The AAV capsid protein of any of claims 1-63 wherein the AAV is AAV9.

65. The AAV capsid protein of any of claims 1-64 wherein the AAV is provided in SEQ ID NO: 1.

66. The AAV capsid protein of any of claims 1-65 wherein the insertion is between amino acid 588 and amino acid 589.

67. The AAV capsid protein of any one of claims 1-66, wherein 60 copies of the AAV capsid protein are assembled into the AAV capsid.

68. The AAV capsid protein of any of claims 1-67, wherein the AAV capsid protein is present in VP1, VP2, and VP3 of the AAV capsid.

69. The AAV capsid protein of any of claims 1-68, wherein the AAV capsid protein is characterized by at least one of an increased transduction enrichment when measured in cardiovascular system in a subject when delivered to the subject systemically.

70. The AAV capsid protein any of claims 1-69, wherein the AAV capsid protein further comprises an amino acid substitution comprising A589N or Q590P.

71. An AAV capsid comprising an AAV capsid protein of any of claims 1-70.

72. The AAV capsid of claim 71, wherein the AAV capsid is chimeric.

73. The AAV capsid of any of claims 71-72 that is isolated and purified.

74. The AAV capsid of any of claims 71-73 formulated as a pharmaceutical formulation for systemic administration to treat a disease or a condition of the heart, the pharmaceutical formulation further comprising a pharmaceutically acceptable carrier.

75. The AAV capsid protein of any of claims 1-70 wherein the capsid protein is expressed in the cardiovascular system.

76. The AAV capsid protein of claim 75, wherein the cardiovascular system comprises a cardiac cell.

77. The AAV capsid of any one of claims 75-76, wherein the cardiovascular system comprises a tissue that is selected from heart.

78. A recombinant vector comprising a nucleic acid encoding the AAV capsid protein of any one of claims 1-70.

79. A kit comprising:
a) a first vector comprising the recombinant vector of claim 78;
b) a second vector encoding a helper virus protein; and
c) a third vector comprising a therapeutic nucleic acid encoding a therapeutic gene expression product.

80. A method of treating a disease or condition in a subject comprising administering a therapeutically effective amount of a pharmaceutical formulation comprising the AAV capsid protein of any one of claims 1-70.

81. The method of claim 80, wherein the disease or the condition is a disease or a condition of a heart of the subject.

82. A method of manufacturing a recombinant AAV particle from the AAV capsid of any one of claims 1-70, the method comprising:
a. introducing into a cell a nucleic acid comprising:
i.a first nucleic acid sequence encoding a therapeutic gene expression product;
ii.a second nucleic acid sequence encoding a recombinant viral genome comprising a capsid (Cap) gene modified to express the AAV capsid of any one of claims 1-70; and
iii.a third nucleic acid sequence encoding an AAV helper virus genome; and
b. assembling the recombinant AAV particle, the recombinant AAV particle comprising the AAV capsid encapsidating the first nucleic acid.

83. The AAV capsid protein of any of claims 1-70 wherein the AAV capsid protein is characterized by an increased transduction enrichment when measured in a tissue in a subject when delivered to the subject systemically.

84. The AAV capsid protein of any of claims 1-70 wherein the tissue is heart or cardiac tissue.

85. An AAV particle comprising an AAV capsid protein of any of claims 1-70 and a viral genome.

86. A peptide comprising an amino acid sequence selected from DLSRLSL, KLDPLLM, TPKHLLI, PDQRRTD, RHDPLVM, SHFSGRN, VSAVRDR, VGVPVVL, LLPSNYL, QLQITKE, LQVTSVL, and VFSGVSS.

87. A recombinant adeno-associated virus (rAAV) comprising a capsid comprising any one of KGGLPFI, DLSRLSL, RSGHPFV, RADLHFQ, LQGHILL, KLDPLLM, IGGHYLH, RAGVHFV, LGGQILI, TPKHLLI, RNGEHFK, KDSIPFR, PDQRRTD, RHDPLVM, DNYTRHT, RSGLHLM, and TNSVRVV.


SEQUENCE LISTING (1209 sequences)

SEQ ID NO: 1 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - skipped

SEQ ID NO: 3 - protein, 7 aa, synthetic construct
KGGLPFI

SEQ ID NO: 4 - protein, 7 aa, synthetic construct
IGGHYLH

SEQ ID NO: 5 - protein, 7 aa, synthetic construct
LGGQILI

SEQ ID NO: 6 - protein, 7 aa, synthetic construct
LGGGFSE

SEQ ID NO: 7 - protein, 7 aa, synthetic construct
IGGNLLK

SEQ ID NO: 8 - protein, 7 aa, synthetic construct
KGGETSS

SEQ ID NO: 9 - protein, 7 aa, synthetic construct
VGGSTYP

SEQ ID NO: 10 - protein, 7 aa, synthetic construct
GGGLAST

SEQ ID NO: 11 - protein, 11 aa, synthetic construct
AQKGGLPFIAQ

SEQ ID NO: 12 - protein, 11 aa, synthetic construct
AQIGGHYLHAQ

SEQ ID NO: 13 - protein, 11 aa, synthetic construct
AQLGGQILIAQ

SEQ ID NO: 14 - protein, 11 aa, synthetic construct
AQLGGGFSEAQ

SEQ ID NO: 15 - protein, 11 aa, synthetic construct
AQIGGNLLKAQ

SEQ ID NO: 16 - protein, 11 aa, synthetic construct
AQKGGETSSAQ

SEQ ID NO: 17 - protein, 11 aa, synthetic construct
AQVGGSTYPAQ

SEQ ID NO: 18 - protein, 11 aa, synthetic construct
AQGGGLASTAQ

SEQ ID NO: 19 - skipped

SEQ ID NO: 20 - protein, 7 aa, synthetic construct
RSGHPFV

SEQ ID NO: 21 - protein, 7 aa, synthetic construct
RSGLHLM

SEQ ID NO: 22 - protein, 7 aa, synthetic construct
RSELHFI

SEQ ID NO: 23 - protein, 7 aa, synthetic construct
RSQTSTE

SEQ ID NO: 24 - protein, 7 aa, synthetic construct
RSISDRV

SEQ ID NO: 25 - protein, 7 aa, synthetic construct
RSTTDAS

SEQ ID NO: 26 - protein, 7 aa, synthetic construct
RSNQESV

SEQ ID NO: 27 - protein, 11 aa, synthetic construct
AQRSGHPFVAQ

SEQ ID NO: 28 - protein, 11 aa, synthetic construct
AQRSGLHLMAQ

SEQ ID NO: 29 - protein, 11 aa, synthetic construct
AQRSELHFIAQ

SEQ ID NO: 30 - protein, 11 aa, synthetic construct
AQRSQTSTEAQ

SEQ ID NO: 31 - protein, 11 aa, synthetic construct
AQRSISDRVAQ

SEQ ID NO: 32 - protein, 11 aa, synthetic construct
AQRSTTDASAQ

SEQ ID NO: 33 - protein, 11 aa, synthetic construct
AQRSNQESVAQ

SEQ ID NO: 34 - skipped

SEQ ID NO: 35 - protein, 7 aa, synthetic construct
RSGHPFV

SEQ ID NO: 36 - protein, 7 aa, synthetic construct
RNGEHFK

SEQ ID NO: 37 - protein, 7 aa, synthetic construct
RDGHYLL

SEQ ID NO: 38 - protein, 7 aa, synthetic construct
RNGEFFH

SEQ ID NO: 39 - protein, 7 aa, synthetic construct
REGMPFL

SEQ ID NO: 40 - protein, 7 aa, synthetic construct
RAGVHFV

SEQ ID NO: 41 - protein, 7 aa, synthetic construct
RSGLHLM

SEQ ID NO: 42 - protein, 7 aa, synthetic construct
RIGVHTS

SEQ ID NO: 43 - protein, 7 aa, synthetic construct
RIGSVTQ

SEQ ID NO: 44 - protein, 7 aa, synthetic construct
RVGSLTD

SEQ ID NO: 45 - skipped

SEQ ID NO: 46 - protein, 7 aa, synthetic construct
RSGHPFV

SEQ ID NO: 47 - protein, 7 aa, synthetic construct
RDGHYLL

SEQ ID NO: 48 - protein, 7 aa, synthetic construct
LQGHILL

SEQ ID NO: 49 - protein, 7 aa, synthetic construct
IGGHYLH

SEQ ID NO: 50 - protein, 11 aa, synthetic construct
AQRSGHPFVAQ

SEQ ID NO: 51 - protein, 11 aa, synthetic construct
AQRDGHYLLAQ

SEQ ID NO: 52 - protein, 11 aa, synthetic construct
AQLQGHILLAQ

SEQ ID NO: 53 - protein, 11 aa, synthetic construct
AQIGGHYLHAQ

SEQ ID NO: 54 - skipped

SEQ ID NO: 55 - protein, 7 aa, synthetic construct
RADLHFQ

SEQ ID NO: 56 - protein, 7 aa, synthetic construct
RAGVHFV

SEQ ID NO: 57 - protein, 7 aa, synthetic construct
RNGEHFK

SEQ ID NO: 58 - protein, 7 aa, synthetic construct
RSELHFI

SEQ ID NO: 59 - protein, 11 aa, synthetic construct
AQRADLHFQAQ

SEQ ID NO: 60 - protein, 11 aa, synthetic construct
AQRAGVHFVAQ

SEQ ID NO: 61 - protein, 11 aa, synthetic construct
AQRNGEHFKAQ

SEQ ID NO: 62 - protein, 11 aa, synthetic construct
AQRSELHFIAQ

SEQ ID NO: 63 - skipped

SEQ ID NO: 64 - protein, 7 aa, synthetic construct
LQGHILL

SEQ ID NO: 65 - protein, 7 aa, synthetic construct
REQGYLL

SEQ ID NO: 66 - protein, 7 aa, synthetic construct
RDGHYLL

SEQ ID NO: 67 - protein, 7 aa, synthetic construct
YTGTSLL

SEQ ID NO: 68 - protein, 7 aa, synthetic construct
AKAVTLL

SEQ ID NO: 69 - protein, 7 aa, synthetic construct
AFGDSLL

SEQ ID NO: 70 - protein, 11 aa, synthetic construct
AQLQGHILLAQ

SEQ ID NO: 71 - protein, 11 aa, synthetic construct
AQREQGYLLAQ

SEQ ID NO: 72 - protein, 11 aa, synthetic construct
AQRDGHYLLAQ

SEQ ID NO: 73 - protein, 11 aa, synthetic construct
AQYTGTSLLAQ

SEQ ID NO: 74 - protein, 11 aa, synthetic construct
AQAKAVTLLAQ

SEQ ID NO: 75 - protein, 11 aa, synthetic construct
AQAFGDSLLAQ

SEQ ID NO: 76 - skipped

SEQ ID NO: 77 - protein, 11 aa, synthetic construct
AQVFSGVSSAQ

SEQ ID NO: 78 - protein, 11 aa, synthetic construct
AQQLQHGSSAQ

SEQ ID NO: 79 - protein, 11 aa, synthetic construct
AQHMASMSSAQ

SEQ ID NO: 80 - protein, 11 aa, synthetic construct
AQSSLTLSSAQ

SEQ ID NO: 81 - protein, 11 aa, synthetic construct
AQSVPSSSSAQ

SEQ ID NO: 82 - protein, 11 aa, synthetic construct
AQITPSVSSAQ

SEQ ID NO: 83 - protein, 11 aa, synthetic construct
AQMTVQVSSAQ

SEQ ID NO: 84 - protein, 11 aa, synthetic construct
AQSHLSVSSAQ

SEQ ID NO: 85 - protein, 11 aa, synthetic construct
AQYPHQTSSAQ

SEQ ID NO: 86 - protein, 11 aa, synthetic construct
AQPQGGSSSAQ

SEQ ID NO: 87 - protein, 11 aa, synthetic construct
AQFRSSLSSAQ

SEQ ID NO: 88 - protein, 11 aa, synthetic construct
AQISVSISSAQ

SEQ ID NO: 89 - protein, 11 aa, synthetic construct
AQETQRGSSAQ

SEQ ID NO: 90 - protein, 11 aa, synthetic construct
AQIHPPSSSAQ

SEQ ID NO: 91 - protein, 11 aa, synthetic construct
AQTSAITSSAQ

SEQ ID NO: 92 - protein, 11 aa, synthetic construct
AQFKSQSSSAQ

SEQ ID NO: 93 - protein, 11 aa, synthetic construct
AQASHSTSSAQ

SEQ ID NO: 94 - protein, 11 aa, synthetic construct
AQAVMRDSSAQ

SEQ ID NO: 95 - protein, 11 aa, synthetic construct
AQKGGETSSAQ

SEQ ID NO: 96 - protein, 11 aa, synthetic construct
AQVVSVTSSAQ

SEQ ID NO: 97 - protein, 11 aa, synthetic construct
AQLISIRSSAQ

SEQ ID NO: 98 - protein, 11 aa, synthetic construct
AQNSLGLSSAQ

SEQ ID NO: 99 - protein, 11 aa, synthetic construct
AQHLAQNSSAQ

SEQ ID NO: 100 - protein, 7 aa, synthetic construct
VFSGVSS

SEQ ID NO: 101 - protein, 7 aa, synthetic construct
QLQHGSS

SEQ ID NO: 102 - protein, 7 aa, synthetic construct
HMASMSS

SEQ ID NO: 103 - protein, 7 aa, synthetic construct
SSLTLSS

SEQ ID NO: 104 - protein, 7 aa, synthetic construct
SVPSSSS

SEQ ID NO: 105 - protein, 7 aa, synthetic construct
ITPSVSS

SEQ ID NO: 106 - protein, 7 aa, synthetic construct
MTVQVSS

SEQ ID NO: 107 - protein, 7 aa, synthetic construct
SHLSVSS

SEQ ID NO: 108 - protein, 7 aa, synthetic construct
YPHQTSS

SEQ ID NO: 109 - protein, 7 aa, synthetic construct
PQGGSSS

SEQ ID NO: 110 - protein, 7 aa, synthetic construct
FRSSLSS

SEQ ID NO: 111 - protein, 7 aa, synthetic construct
ISVSISS

SEQ ID NO: 112 - protein, 7 aa, synthetic construct
ETQRGSS

SEQ ID NO: 113 - protein, 7 aa, synthetic construct
IHPPSSS

SEQ ID NO: 114 - protein, 7 aa, synthetic construct
TSAITSS

SEQ ID NO: 115 - protein, 7 aa, synthetic construct
FKSQSSS

SEQ ID NO: 116 - protein, 7 aa, synthetic construct
ASHSTSS

SEQ ID NO: 117 - protein, 7 aa, synthetic construct
AVMRDSS

SEQ ID NO: 118 - protein, 7 aa, synthetic construct
KGGETSS

SEQ ID NO: 119 - protein, 7 aa, synthetic construct
VVSVTSS

SEQ ID NO: 120 - protein, 7 aa, synthetic construct
LISIRSS

SEQ ID NO: 121 - protein, 7 aa, synthetic construct
NSLGLSS

SEQ ID NO: 122 - protein, 7 aa, synthetic construct
HLAQNSS

SEQ ID NO: 123 - skipped

SEQ ID NO: 124 - protein, 6 aa, synthetic construct
DLSRLS

SEQ ID NO: 125 - protein, 7 aa, synthetic construct
DLHKSVA

SEQ ID NO: 126 - protein, 7 aa, synthetic construct
DLGNLKV

SEQ ID NO: 127 - protein, 7 aa, synthetic construct
DLTPHKD

SEQ ID NO: 128 - protein, 7 aa, synthetic construct
DLQRSDL

SEQ ID NO: 129 - protein, 7 aa, synthetic construct
DLGSRRE

SEQ ID NO: 130 - protein, 11 aa, synthetic construct
AQDLSRLSLAQ

SEQ ID NO: 131 - protein, 11 aa, synthetic construct
AQDLHKSVAAQ

SEQ ID NO: 132 - protein, 11 aa, synthetic construct
AQDLGNLKVAQ

SEQ ID NO: 133 - protein, 11 aa, synthetic construct
AQDLTPHKDAQ

SEQ ID NO: 134 - protein, 11 aa, synthetic construct
AQDLQRSDLAQ

SEQ ID NO: 135 - protein, 11 aa, synthetic construct
AQDLGSRREAQ

SEQ ID NO: 136 - protein, 7 aa, synthetic construct
DLSRLSL

SEQ ID NO: 137 - protein, 7 aa, synthetic construct
KLDPLLM

SEQ ID NO: 138 - protein, 7 aa, synthetic construct
TPKHLLI

SEQ ID NO: 139 - protein, 7 aa, synthetic construct
PDQRRTD

SEQ ID NO: 140 - protein, 7 aa, synthetic construct
RHDPLVM

SEQ ID NO: 141 - protein, 7 aa, synthetic construct
SHFSGRN

SEQ ID NO: 142 - protein, 7 aa, synthetic construct
VSAVRDR

SEQ ID NO: 143 - protein, 7 aa, synthetic construct
VGVPVVL

SEQ ID NO: 144 - protein, 7 aa, synthetic construct
LLPSNYL

SEQ ID NO: 145 - protein, 7 aa, synthetic construct
QLQITKE

SEQ ID NO: 146 - protein, 7 aa, synthetic construct
LQVTSVL

SEQ ID NO: 147 - protein, 7 aa, synthetic construct
VFSGVSS

SEQ ID NO: 148 - protein, 11 aa, synthetic construct
AQDLSRLSLAQ

SEQ ID NO: 149 - protein, 11 aa, synthetic construct
AQKLDPLLMAQ

SEQ ID NO: 150 - protein, 11 aa, synthetic construct
AQTPKHLLIAQ

SEQ ID NO: 151 - protein, 11 aa, synthetic construct
AQPDQRRTDAQ

SEQ ID NO: 152 - protein, 11 aa, synthetic construct
AQRHDPLVMAQ

SEQ ID NO: 153 - protein, 11 aa, synthetic construct
AQSHFSGRNAQ

SEQ ID NO: 154 - protein, 11 aa, synthetic construct
AQVSAVRDRAQ

SEQ ID NO: 155 - protein, 11 aa, synthetic construct
AQVGVPVVLAQ

SEQ ID NO: 156 - protein, 11 aa, synthetic construct
AQLLPSNYLAQ

SEQ ID NO: 157 - protein, 11 aa, synthetic construct
AQQLQITKEAQ

SEQ ID NO: 158 - protein, 11 aa, synthetic construct
AQLQVTSVLAQ

SEQ ID NO: 159 - protein, 11 aa, synthetic construct
AQVFSGVSSAQ

SEQ ID NO: 160 - protein, 7 aa, synthetic construct
KGGLPFI

SEQ ID NO: 161 - protein, 7 aa, synthetic construct
DLSRLSL

SEQ ID NO: 162 - protein, 7 aa, synthetic construct
RSGHPFV

SEQ ID NO: 163 - protein, 7 aa, synthetic construct
RADLHFQ

SEQ ID NO: 164 - protein, 7 aa, synthetic construct
LQGHILL

SEQ ID NO: 165 - protein, 7 aa, synthetic construct
KLDPLLM

SEQ ID NO: 166 - protein, 7 aa, synthetic construct
IGGHYLH

SEQ ID NO: 167 - protein, 7 aa, synthetic construct
RAGVHFV

SEQ ID NO: 168 - protein, 7 aa, synthetic construct
LGGQILI

SEQ ID NO: 169 - protein, 7 aa, synthetic construct
TPKHLLI

SEQ ID NO: 170 - protein, 7 aa, synthetic construct
RNGEHFK

SEQ ID NO: 171 - protein, 7 aa, synthetic construct
KDSIPFR

SEQ ID NO: 172 - protein, 7 aa, synthetic construct
PDQRRTD

SEQ ID NO: 173 - protein, 7 aa, synthetic construct
RHDPLVM

SEQ ID NO: 174 - protein, 7 aa, synthetic construct
DNYTRHT

SEQ ID NO: 175 - protein, 7 aa, synthetic construct
RSGLHLM

SEQ ID NO: 176 - protein, 7 aa, synthetic construct
TNSVRVV

SEQ ID NO: 177 - protein, 11 aa, synthetic construct
AQKGGLPFIAQ

SEQ ID NO: 178 - protein, 11 aa, synthetic construct
AQDLSRLSLAQ

SEQ ID NO: 179 - protein, 11 aa, synthetic construct
AQRSGHPFVAQ

SEQ ID NO: 180 - protein, 11 aa, synthetic construct
AQRADLHFQAQ

SEQ ID NO: 181 - protein, 11 aa, synthetic construct
AQLQGHILLAQ

SEQ ID NO: 182 - protein, 11 aa, synthetic construct
AQKLDPLLMAQ

SEQ ID NO: 183 - protein, 11 aa, synthetic construct
AQIGGHYLHAQ

SEQ ID NO: 184 - protein, 11 aa, synthetic construct
AQRAGVHFVAQ

SEQ ID NO: 185 - protein, 11 aa, synthetic construct
AQLGGQILIAQ

SEQ ID NO: 186 - protein, 11 aa, synthetic construct
AQTPKHLLIAQ

SEQ ID NO: 187 - protein, 11 aa, synthetic construct
AQRNGEHFKAQ

SEQ ID NO: 188 - protein, 11 aa, synthetic construct
AQKDSIPFRAQ

SEQ ID NO: 189 - protein, 11 aa, synthetic construct
AQPDQRRTDAQ

SEQ ID NO: 190 - protein, 11 aa, synthetic construct
AQRHDPLVMAQ

SEQ ID NO: 191 - protein, 11 aa, synthetic construct
AQDNYTRHTAQ

SEQ ID NO: 192 - protein, 11 aa, synthetic construct
AQRSGLHLMAQ

SEQ ID NO: 193 - protein, 11 aa, synthetic construct
AQTNSVRVVAQ

SEQ ID NO: 194 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQKGGLPFIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 195 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQDLSRLSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 196 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRSGHPFVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 197 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRADLHFQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 198 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLQGHILLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 199 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQKLDPLLMAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 200 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 201 - protein, 11 aa, synthetic construct
AQKGGLPFIAQ

SEQ ID NO: 202 - protein, 11 aa, synthetic construct
AQDLSRLSLAQ

SEQ ID NO: 203 - protein, 11 aa, synthetic construct
AQRNGHILIAQ

SEQ ID NO: 204 - protein, 11 aa, synthetic construct
AQRSGHPFVAQ

SEQ ID NO: 205 - protein, 11 aa, synthetic construct
AQRADLHFQAQ

SEQ ID NO: 206 - protein, 11 aa, synthetic construct
AQLQGHILLAQ

SEQ ID NO: 207 - protein, 11 aa, synthetic construct
AQKLDPLLMAQ

SEQ ID NO: 208 - protein, 11 aa, synthetic construct
AQIGGHYLHAQ

SEQ ID NO: 209 - protein, 11 aa, synthetic construct
AQKPPGYLLAQ

SEQ ID NO: 210 - protein, 11 aa, synthetic construct
AQLSGQILIAQ

SEQ ID NO: 211 - protein, 11 aa, synthetic construct
AQRAGVHFVAQ

SEQ ID NO: 212 - protein, 11 aa, synthetic construct
AQLGGQILIAQ

SEQ ID NO: 213 - protein, 11 aa, synthetic construct
AQTPKHLLIAQ

SEQ ID NO: 214 - protein, 11 aa, synthetic construct
AQRNGEHFKAQ

SEQ ID NO: 215 - protein, 11 aa, synthetic construct
AQKDSIPFRAQ

SEQ ID NO: 216 - protein, 11 aa, synthetic construct
AQPDQRRTDAQ

SEQ ID NO: 217 - protein, 11 aa, synthetic construct
AQRHDPLVMAQ

SEQ ID NO: 218 - protein, 11 aa, synthetic construct
AQDNYTRHTAQ

SEQ ID NO: 219 - protein, 11 aa, synthetic construct
AQRSGLHLMAQ

SEQ ID NO: 220 - protein, 11 aa, synthetic construct
AQTNSVRVVAQ

SEQ ID NO: 221 - protein, 11 aa, synthetic construct
AQREQGYLLAQ

SEQ ID NO: 222 - protein, 11 aa, synthetic construct
AQGNVVRPLAQ

SEQ ID NO: 223 - protein, 11 aa, synthetic construct
AQFNTTRNSAQ

SEQ ID NO: 224 - protein, 11 aa, synthetic construct
AQRDGHYLLAQ

SEQ ID NO: 225 - protein, 11 aa, synthetic construct
AQINATRLVAQ

SEQ ID NO: 226 - protein, 11 aa, synthetic construct
AQRSELHFIAQ

SEQ ID NO: 227 - protein, 11 aa, synthetic construct
AQRPDGPFIAQ

SEQ ID NO: 228 - protein, 11 aa, synthetic construct
AQSHFSGRNAQ

SEQ ID NO: 229 - protein, 11 aa, synthetic construct
AQVSAVRDRAQ

SEQ ID NO: 230 - protein, 11 aa, synthetic construct
AQQSMKLGLAQ

SEQ ID NO: 231 - protein, 11 aa, synthetic construct
AQVGVPVVLAQ

SEQ ID NO: 232 - protein, 11 aa, synthetic construct
AQLLPSNYLAQ

SEQ ID NO: 233 - protein, 11 aa, synthetic construct
AQQLQITKEAQ

SEQ ID NO: 234 - protein, 11 aa, synthetic construct
AQLQVTSVLAQ

SEQ ID NO: 235 - protein, 11 aa, synthetic construct
AQVFSGVSSAQ

SEQ ID NO: 236 - protein, 11 aa, synthetic construct
AQLTKPSTVTQ

SEQ ID NO: 237 - protein, 11 aa, synthetic construct
AQGDSTRVSAQ

SEQ ID NO: 238 - protein, 11 aa, synthetic construct
AQLVQRVPSAQ

SEQ ID NO: 239 - protein, 11 aa, synthetic construct
AQANSRGALAQ

SEQ ID NO: 240 - protein, 11 aa, synthetic construct
AQFLSHSNIAQ

SEQ ID NO: 241 - protein, 11 aa, synthetic construct
AQNIRGGLVAQ

SEQ ID NO: 242 - protein, 11 aa, synthetic construct
AQRDAVYLHAQ

SEQ ID NO: 243 - protein, 11 aa, synthetic construct
AQGVFGTGAAQ

SEQ ID NO: 244 - protein, 11 aa, synthetic construct
AQSQTRSLSAQ

SEQ ID NO: 245 - protein, 11 aa, synthetic construct
AQLNSIRNLAQ

SEQ ID NO: 246 - protein, 11 aa, synthetic construct
AQMTQGGNNAQ

SEQ ID NO: 247 - protein, 11 aa, synthetic construct
AQYLHVRDSAQ

SEQ ID NO: 248 - protein, 11 aa, synthetic construct
AQPLQPLHTAQ

SEQ ID NO: 249 - protein, 11 aa, synthetic construct
AQVQHLGSQAQ

SEQ ID NO: 250 - protein, 11 aa, synthetic construct
AQVLLHSTNAQ

SEQ ID NO: 251 - protein, 11 aa, synthetic construct
AQDRHSTLFAQ

SEQ ID NO: 252 - protein, 11 aa, synthetic construct
AQLSQRSDMAQ

SEQ ID NO: 253 - protein, 11 aa, synthetic construct
AQRIGSVTQAQ

SEQ ID NO: 254 - protein, 11 aa, synthetic construct
AQDTIFSRSAQ

SEQ ID NO: 255 - protein, 11 aa, synthetic construct
AQSILSPNSAQ

SEQ ID NO: 256 - protein, 11 aa, synthetic construct
AQGRIHTETAQ

SEQ ID NO: 257 - protein, 11 aa, synthetic construct
AQGISTSTGAQ

SEQ ID NO: 258 - protein, 11 aa, synthetic construct
AQLGSYTTHAQ

SEQ ID NO: 259 - protein, 11 aa, synthetic construct
AQTTYTSPGAQ

SEQ ID NO: 260 - protein, 11 aa, synthetic construct
AQTRAGDSNAQ

SEQ ID NO: 261 - protein, 11 aa, synthetic construct
AQTSAVTIVAQ

SEQ ID NO: 262 - protein, 11 aa, synthetic construct
AQGRGDSFLAQ

SEQ ID NO: 263 - protein, 11 aa, synthetic construct
AQINGVRYLAQ

SEQ ID NO: 264 - protein, 11 aa, synthetic construct
AQDLHKSVAAQ

SEQ ID NO: 265 - protein, 11 aa, synthetic construct
AQQYQSNMYAQ

SEQ ID NO: 266 - protein, 11 aa, synthetic construct
AQQSSRLYEAQ

SEQ ID NO: 267 - protein, 11 aa, synthetic construct
AQLSLGTREAQ

SEQ ID NO: 268 - protein, 11 aa, synthetic construct
AQLEPILRGAQ

SEQ ID NO: 269 - protein, 11 aa, synthetic construct
AQEARLNLAAQ

SEQ ID NO: 270 - protein, 11 aa, synthetic construct
AQPQNSGHGAQ

SEQ ID NO: 271 - protein, 11 aa, synthetic construct
AQINQTSTGAQ

SEQ ID NO: 272 - protein, 11 aa, synthetic construct
AQSDVQRSYAQ

SEQ ID NO: 273 - protein, 11 aa, synthetic construct
AQKTGTHVSAQ

SEQ ID NO: 274 - protein, 11 aa, synthetic construct
AQEQVLKSAAQ

SEQ ID NO: 275 - protein, 11 aa, synthetic construct
AQISTYALAAQ

SEQ ID NO: 276 - protein, 11 aa, synthetic construct
AQDNHLKNMAQ

SEQ ID NO: 277 - protein, 11 aa, synthetic construct
AQLVSTYHSAQ

SEQ ID NO: 278 - protein, 11 aa, synthetic construct
AQIKAMPEYAQ

SEQ ID NO: 279 - protein, 11 aa, synthetic construct
AQSSYPTTMAQ

SEQ ID NO: 280 - protein, 11 aa, synthetic construct
AQETVRLAVAQ

SEQ ID NO: 281 - protein, 11 aa, synthetic construct
AQLATSRTLAQ

SEQ ID NO: 282 - protein, 11 aa, synthetic construct
AQVSGTSSGAQ

SEQ ID NO: 283 - protein, 11 aa, synthetic construct
AQLSFSNNLAQ

SEQ ID NO: 284 - protein, 11 aa, synthetic construct
AQGPGSITSAQ

SEQ ID NO: 285 - protein, 11 aa, synthetic construct
AQTPKAGTSAQ

SEQ ID NO: 286 - protein, 11 aa, synthetic construct
AQSTTASNLAQ

SEQ ID NO: 287 - protein, 11 aa, synthetic construct
AQGLRSTGIAQ

SEQ ID NO: 288 - protein, 11 aa, synthetic construct
AQAQRPPTLAQ

SEQ ID NO: 289 - protein, 11 aa, synthetic construct
AQDTARANSAQ

SEQ ID NO: 290 - protein, 11 aa, synthetic construct
AQLTLGSSEAQ

SEQ ID NO: 291 - protein, 11 aa, synthetic construct
AQQHNSLGSAQ

SEQ ID NO: 292 - protein, 11 aa, synthetic construct
AQTLAKTVLAQ

SEQ ID NO: 293 - protein, 11 aa, synthetic construct
AQLTRSNDPAQ

SEQ ID NO: 294 - protein, 11 aa, synthetic construct
AQIHGNSTIAQ

SEQ ID NO: 295 - protein, 11 aa, synthetic construct
AQPHTSRLEAQ

SEQ ID NO: 296 - protein, 11 aa, synthetic construct
AQAPVHTPGAQ

SEQ ID NO: 297 - protein, 11 aa, synthetic construct
AQQRVSLGEAQ

SEQ ID NO: 298 - protein, 11 aa, synthetic construct
AQQTQSVISAQ

SEQ ID NO: 299 - protein, 11 aa, synthetic construct
AQFPISTTNAQ

SEQ ID NO: 300 - protein, 11 aa, synthetic construct
AQRNGEFFHAQ

SEQ ID NO: 301 - protein, 11 aa, synthetic construct
AQVNKVVGLAQ

SEQ ID NO: 302 - protein, 11 aa, synthetic construct
AQQDTLRNPAQ

SEQ ID NO: 303 - protein, 11 aa, synthetic construct
AQIISSATAAQ

SEQ ID NO: 304 - protein, 11 aa, synthetic construct
AQSSNRMSTAQ

SEQ ID NO: 305 - protein, 11 aa, synthetic construct
AQLTTLTTAAQ

SEQ ID NO: 306 - protein, 11 aa, synthetic construct
AQFPQTNYGAQ

SEQ ID NO: 307 - protein, 11 aa, synthetic construct
AQTASKDAYAQ

SEQ ID NO: 308 - protein, 11 aa, synthetic construct
AQYTMSTSMAQ

SEQ ID NO: 309 - protein, 11 aa, synthetic construct
AQQLDGNRSAQ

SEQ ID NO: 310 - protein, 11 aa, synthetic construct
AQTTSGHSMAQ

SEQ ID NO: 311 - protein, 11 aa, synthetic construct
AQQSSHVMSAQ

SEQ ID NO: 312 - protein, 11 aa, synthetic construct
AQDSRSTYQAQ

SEQ ID NO: 313 - protein, 11 aa, synthetic construct
AQRADSHIFAQ

SEQ ID NO: 314 - protein, 11 aa, synthetic construct
AQMTSPGRDAQ

SEQ ID NO: 315 - protein, 11 aa, synthetic construct
AQFANTHPIAQ

SEQ ID NO: 316 - protein, 11 aa, synthetic construct
AQALSRSSEAQ

SEQ ID NO: 317 - protein, 11 aa, synthetic construct
AQGIALRSTAQ

SEQ ID NO: 318 - protein, 11 aa, synthetic construct
AQDNAKLHGAQ

SEQ ID NO: 319 - protein, 11 aa, synthetic construct
AQLTSRTEMAQ

SEQ ID NO: 320 - protein, 11 aa, synthetic construct
AQWSGSPYAAQ

SEQ ID NO: 321 - protein, 11 aa, synthetic construct
AQVLSKSPAAQ

SEQ ID NO: 322 - protein, 11 aa, synthetic construct
AQYGRSPESAQ

SEQ ID NO: 323 - protein, 11 aa, synthetic construct
AQLLKGASEAQ

SEQ ID NO: 324 - protein, 11 aa, synthetic construct
AQTAKTETNAQ

SEQ ID NO: 325 - protein, 11 aa, synthetic construct
AQQTYSTNPAQ

SEQ ID NO: 326 - protein, 11 aa, synthetic construct
AQTLSSGKQAQ

SEQ ID NO: 327 - protein, 11 aa, synthetic construct
AQKFSNGMEAQ

SEQ ID NO: 328 - protein, 11 aa, synthetic construct
AQYVTHERNAQ

SEQ ID NO: 329 - protein, 11 aa, synthetic construct
AQVFGRTDPAQ

SEQ ID NO: 330 - protein, 11 aa, synthetic construct
AQSAVKPGEAQ

SEQ ID NO: 331 - protein, 11 aa, synthetic construct
AQIFQSAVSAQ

SEQ ID NO: 332 - protein, 11 aa, synthetic construct
AQSYNTSQMAQ

SEQ ID NO: 333 - protein, 11 aa, synthetic construct
AQMSMSLGGAQ

SEQ ID NO: 334 - protein, 11 aa, synthetic construct
AQAGVLRQVAQ

SEQ ID NO: 335 - protein, 11 aa, synthetic construct
AQVVHTMQAAQ

SEQ ID NO: 336 - protein, 11 aa, synthetic construct
AQLTGRTNEAQ

SEQ ID NO: 337 - protein, 11 aa, synthetic construct
AQMHSNTAGAQ

SEQ ID NO: 338 - protein, 11 aa, synthetic construct
AQQGTSLIPAQ

SEQ ID NO: 339 - protein, 11 aa, synthetic construct
AQSSSQVGMAQ

SEQ ID NO: 340 - protein, 11 aa, synthetic construct
AQTSDRVLPAQ

SEQ ID NO: 341 - protein, 11 aa, synthetic construct
AQYKSDTTGAQ

SEQ ID NO: 342 - protein, 11 aa, synthetic construct
AQGFDSTRSAQ

SEQ ID NO: 343 - protein, 11 aa, synthetic construct
AQRAPSTSVAQ

SEQ ID NO: 344 - protein, 11 aa, synthetic construct
AQQRSSDSLAQ

SEQ ID NO: 345 - protein, 11 aa, synthetic construct
AQGSGTLHPAQ

SEQ ID NO: 346 - protein, 11 aa, synthetic construct
AQTQVLSLSAQ

SEQ ID NO: 347 - protein, 11 aa, synthetic construct
AQHSVVSVGAQ

SEQ ID NO: 348 - protein, 11 aa, synthetic construct
AQVSSNMGSAQ

SEQ ID NO: 349 - protein, 11 aa, synthetic construct
AQQLQHGSSAQ

SEQ ID NO: 350 - protein, 11 aa, synthetic construct
AQGRQDAYGAQ

SEQ ID NO: 351 - protein, 11 aa, synthetic construct
AQSRSNTVTAQ

SEQ ID NO: 352 - protein, 11 aa, synthetic construct
AQHMASMSSAQ

SEQ ID NO: 353 - protein, 11 aa, synthetic construct
AQSDTHRGAAQ

SEQ ID NO: 354 - protein, 11 aa, synthetic construct
AQRSQTSTEAQ

SEQ ID NO: 355 - protein, 11 aa, synthetic construct
AQGGSHMTAAQ

SEQ ID NO: 356 - protein, 11 aa, synthetic construct
AQPMSMGKDAQ

SEQ ID NO: 357 - protein, 11 aa, synthetic construct
AQTTSRVDHAQ

SEQ ID NO: 358 - protein, 11 aa, synthetic construct
AQSPRSSLTAQ

SEQ ID NO: 359 - protein, 11 aa, synthetic construct
AQPQASHIFAQ

SEQ ID NO: 360 - protein, 11 aa, synthetic construct
AQTMGQATTAQ

SEQ ID NO: 361 - protein, 11 aa, synthetic construct
AQWTGRTIDAQ

SEQ ID NO: 362 - protein, 11 aa, synthetic construct
AQRSISDRVAQ

SEQ ID NO: 363 - protein, 11 aa, synthetic construct
AQNSVRSSLAQ

SEQ ID NO: 364 - protein, 11 aa, synthetic construct
AQSLSYTAGAQ

SEQ ID NO: 365 - protein, 11 aa, synthetic construct
AQRQISSVMAQ

SEQ ID NO: 366 - protein, 11 aa, synthetic construct
AQGTFSHTVAQ

SEQ ID NO: 367 - protein, 11 aa, synthetic construct
AQSSPAHHLAQ

SEQ ID NO: 368 - protein, 11 aa, synthetic construct
AQHQNKVEVAQ

SEQ ID NO: 369 - protein, 11 aa, synthetic construct
AQARVQESLAQ

SEQ ID NO: 370 - protein, 11 aa, synthetic construct
AQEIGKRTDAQ

SEQ ID NO: 371 - protein, 11 aa, synthetic construct
AQTLVHSNNAQ

SEQ ID NO: 372 - protein, 11 aa, synthetic construct
AQFRNTGPAAQ

SEQ ID NO: 373 - protein, 11 aa, synthetic construct
AQLETYERAAQ

SEQ ID NO: 374 - protein, 11 aa, synthetic construct
AQTYGSQTGAQ

SEQ ID NO: 375 - protein, 11 aa, synthetic construct
AQFALSTHPAQ

SEQ ID NO: 376 - protein, 11 aa, synthetic construct
AQSKTSTLAAQ

SEQ ID NO: 377 - protein, 11 aa, synthetic construct
AQSSQLVTAAQ

SEQ ID NO: 378 - protein, 11 aa, synthetic construct
AQEMRVTHTAQ

SEQ ID NO: 379 - protein, 11 aa, synthetic construct
AQESHLPAGAQ

SEQ ID NO: 380 - protein, 11 aa, synthetic construct
AQSAGGGNMAQ

SEQ ID NO: 381 - protein, 11 aa, synthetic construct
AQIINTERTAQ

SEQ ID NO: 382 - protein, 11 aa, synthetic construct
AQLPQAIPKAQ

SEQ ID NO: 383 - protein, 11 aa, synthetic construct
AQLQPLTHSAQ

SEQ ID NO: 384 - protein, 11 aa, synthetic construct
AQTSNLQPSAQ

SEQ ID NO: 385 - protein, 11 aa, synthetic construct
AQLSQPHGDAQ

SEQ ID NO: 386 - protein, 11 aa, synthetic construct
AQTVHRMGEAQ

SEQ ID NO: 387 - protein, 11 aa, synthetic construct
AQNATASISAQ

SEQ ID NO: 388 - protein, 11 aa, synthetic construct
AQIAGSTYIAQ

SEQ ID NO: 389 - protein, 11 aa, synthetic construct
AQLLTSGGQAQ

SEQ ID NO: 390 - protein, 11 aa, synthetic construct
AQVMGSGFVAQ

SEQ ID NO: 391 - protein, 11 aa, synthetic construct
AQNMGSSSPAQ

SEQ ID NO: 392 - protein, 11 aa, synthetic construct
AQYTGTSLLAQ

SEQ ID NO: 393 - protein, 11 aa, synthetic construct
AQSTGSGPLAQ

SEQ ID NO: 394 - protein, 11 aa, synthetic construct
AQILHKSSEAQ

SEQ ID NO: 395 - protein, 11 aa, synthetic construct
AQSQTRTGDAQ

SEQ ID NO: 396 - protein, 11 aa, synthetic construct
AQSHGTSMSAQ

SEQ ID NO: 397 - protein, 11 aa, synthetic construct
AQERNVTQGAQ

SEQ ID NO: 398 - protein, 11 aa, synthetic construct
AQNVTQTATAQ

SEQ ID NO: 399 - protein, 11 aa, synthetic construct
AQRDGLILIAQ

SEQ ID NO: 400 - protein, 11 aa, synthetic construct
AQRGTLTTVAQ

SEQ ID NO: 401 - protein, 11 aa, synthetic construct
AQRLTVQSNAQ

SEQ ID NO: 402 - protein, 11 aa, synthetic construct
AQSARTTNLAQ

SEQ ID NO: 403 - protein, 11 aa, synthetic construct
AQMSVTLTSAQ

SEQ ID NO: 404 - protein, 11 aa, synthetic construct
AQSPPSRTSAQ

SEQ ID NO: 405 - protein, 11 aa, synthetic construct
AQMHSNFSAAQ

SEQ ID NO: 406 - protein, 11 aa, synthetic construct
AQGESSMRDAQ

SEQ ID NO: 407 - protein, 11 aa, synthetic construct
AQRTDHVVAAQ

SEQ ID NO: 408 - protein, 11 aa, synthetic construct
AQTTVSAHSAQ

SEQ ID NO: 409 - protein, 11 aa, synthetic construct
AQLSDQSRNAQ

SEQ ID NO: 410 - protein, 11 aa, synthetic construct
AQPQVRSAVAQ

SEQ ID NO: 411 - protein, 11 aa, synthetic construct
AQTLGPLGMAQ

SEQ ID NO: 412 - protein, 11 aa, synthetic construct
AQYSHSLQGAQ

SEQ ID NO: 413 - protein, 11 aa, synthetic construct
AQSSVAVDRAQ

SEQ ID NO: 414 - protein, 11 aa, synthetic construct
AQQVPASITAQ

SEQ ID NO: 415 - protein, 11 aa, synthetic construct
AQTPLKMHNAQ

SEQ ID NO: 416 - protein, 11 aa, synthetic construct
AQIMSNRSNAQ

SEQ ID NO: 417 - protein, 11 aa, synthetic construct
AQMVTTRVEAQ

SEQ ID NO: 418 - protein, 11 aa, synthetic construct
AQQAKSALPAQ

SEQ ID NO: 419 - protein, 11 aa, synthetic construct
AQLHMSTSPAQ

SEQ ID NO: 420 - protein, 11 aa, synthetic construct
AQFVRDSNMAQ

SEQ ID NO: 421 - protein, 11 aa, synthetic construct
AQIMLSHSPAQ

SEQ ID NO: 422 - protein, 11 aa, synthetic construct
AQLVSLKVTAQ

SEQ ID NO: 423 - protein, 11 aa, synthetic construct
AQLRPSPKEAQ

SEQ ID NO: 424 - protein, 11 aa, synthetic construct
AQRPSLGLPAQ

SEQ ID NO: 425 - protein, 11 aa, synthetic construct
AQSQWISREAQ

SEQ ID NO: 426 - protein, 11 aa, synthetic construct
AQAVGMHKVAQ

SEQ ID NO: 427 - protein, 11 aa, synthetic construct
AQRLSLLLNAQ

SEQ ID NO: 428 - protein, 11 aa, synthetic construct
AQLTRIGHEAQ

SEQ ID NO: 429 - protein, 11 aa, synthetic construct
AQFYTGSLTAQ

SEQ ID NO: 430 - protein, 11 aa, synthetic construct
AQQSYPNTAAQ

SEQ ID NO: 431 - protein, 11 aa, synthetic construct
AQVNTIAGGAQ

SEQ ID NO: 432 - protein, 11 aa, synthetic construct
AQTKSSDHSAQ

SEQ ID NO: 433 - protein, 11 aa, synthetic construct
AQLGSSHNAAQ

SEQ ID NO: 434 - protein, 11 aa, synthetic construct
AQLSNVHVIAQ

SEQ ID NO: 435 - protein, 11 aa, synthetic construct
AQTQVNAAMAQ

SEQ ID NO: 436 - protein, 11 aa, synthetic construct
AQSSLTLSSAQ

SEQ ID NO: 437 - protein, 11 aa, synthetic construct
AQYNYQRLEAQ

SEQ ID NO: 438 - protein, 11 aa, synthetic construct
AQQMTSSIAAQ

SEQ ID NO: 439 - protein, 11 aa, synthetic construct
AQTFQGSSTAQ

SEQ ID NO: 440 - protein, 11 aa, synthetic construct
AQSTRAPGMAQ

SEQ ID NO: 441 - protein, 11 aa, synthetic construct
AQVYSKPESAQ

SEQ ID NO: 442 - protein, 11 aa, synthetic construct
AQSVPTSGNAQ

SEQ ID NO: 443 - protein, 11 aa, synthetic construct
AQIPVNTHSAQ

SEQ ID NO: 444 - protein, 11 aa, synthetic construct
AQPSNRGNEAQ

SEQ ID NO: 445 - protein, 11 aa, synthetic construct
AQDSTSRNHAQ

SEQ ID NO: 446 - protein, 11 aa, synthetic construct
AQHELGRQGAQ

SEQ ID NO: 447 - protein, 11 aa, synthetic construct
AQGRVHVSEAQ

SEQ ID NO: 448 - protein, 11 aa, synthetic construct
AQIPSYSNTAQ

SEQ ID NO: 449 - protein, 11 aa, synthetic construct
AQLIRATGPAQ

SEQ ID NO: 450 - protein, 11 aa, synthetic construct
AQDQSPRTSAQ

SEQ ID NO: 451 - protein, 11 aa, synthetic construct
AQNSAKGLSAQ

SEQ ID NO: 452 - protein, 11 aa, synthetic construct
AQSTVPLANAQ

SEQ ID NO: 453 - protein, 11 aa, synthetic construct
AQYHQSATGAQ

SEQ ID NO: 454 - protein, 11 aa, synthetic construct
AQMPGAITHAQ

SEQ ID NO: 455 - protein, 11 aa, synthetic construct
AQSSIRDNSAQ

SEQ ID NO: 456 - protein, 11 aa, synthetic construct
AQKHLASVGAQ

SEQ ID NO: 457 - protein, 11 aa, synthetic construct
AQSVPSSSSAQ

SEQ ID NO: 458 - protein, 11 aa, synthetic construct
AQLLGASPMAQ

SEQ ID NO: 459 - protein, 11 aa, synthetic construct
AQLGHNSVHAQ

SEQ ID NO: 460 - protein, 11 aa, synthetic construct
AQALNSLASAQ

SEQ ID NO: 461 - protein, 11 aa, synthetic construct
AQAHSTGKEAQ

SEQ ID NO: 462 - protein, 11 aa, synthetic construct
AQHIPVAREAQ

SEQ ID NO: 463 - protein, 11 aa, synthetic construct
AQHRVELTAAQ

SEQ ID NO: 464 - protein, 11 aa, synthetic construct
AQSGNSRLVAQ

SEQ ID NO: 465 - protein, 11 aa, synthetic construct
AQSTTSTHPAQ

SEQ ID NO: 466 - protein, 11 aa, synthetic construct
AQVPPSSAHAQ

SEQ ID NO: 467 - protein, 11 aa, synthetic construct
AQAAHTALMAQ

SEQ ID NO: 468 - protein, 11 aa, synthetic construct
AQITSSHLVAQ

SEQ ID NO: 469 - protein, 11 aa, synthetic construct
AQSNPTNTPAQ

SEQ ID NO: 470 - protein, 11 aa, synthetic construct
AQSVQSGHHAQ

SEQ ID NO: 471 - protein, 11 aa, synthetic construct
AQIMGLTSNAQ

SEQ ID NO: 472 - protein, 11 aa, synthetic construct
AQSKSHEPNAQ

SEQ ID NO: 473 - protein, 11 aa, synthetic construct
AQLVHNTTGAQ

SEQ ID NO: 474 - protein, 11 aa, synthetic construct
AQHNTSTGYAQ

SEQ ID NO: 475 - protein, 11 aa, synthetic construct
AQQLTSTGSAQ

SEQ ID NO: 476 - protein, 11 aa, synthetic construct
AQGLLSTSAAQ

SEQ ID NO: 477 - protein, 11 aa, synthetic construct
AQVTVSSLTAQ

SEQ ID NO: 478 - protein, 11 aa, synthetic construct
AQEGRTMLNAQ

SEQ ID NO: 479 - protein, 11 aa, synthetic construct
AQITPSVSSAQ

SEQ ID NO: 480 - protein, 11 aa, synthetic construct
AQLNSTFRGAQ

SEQ ID NO: 481 - protein, 11 aa, synthetic construct
AQSNVPTATAQ

SEQ ID NO: 482 - protein, 11 aa, synthetic construct
AQQIANAVGAQ

SEQ ID NO: 483 - protein, 11 aa, synthetic construct
AQTALTHSGAQ

SEQ ID NO: 484 - protein, 11 aa, synthetic construct
AQLPVSRGAAQ

SEQ ID NO: 485 - protein, 11 aa, synthetic construct
AQARSVHQVAQ

SEQ ID NO: 486 - protein, 11 aa, synthetic construct
AQIPTNTNIAQ

SEQ ID NO: 487 - protein, 11 aa, synthetic construct
AQIALTSQSAQ

SEQ ID NO: 488 - protein, 11 aa, synthetic construct
AQEINRLMPAQ

SEQ ID NO: 489 - protein, 11 aa, synthetic construct
AQRVGSLTDAQ

SEQ ID NO: 490 - protein, 11 aa, synthetic construct
AQEASGLKMAQ

SEQ ID NO: 491 - protein, 11 aa, synthetic construct
AQVSVSSKSAQ

SEQ ID NO: 492 - protein, 11 aa, synthetic construct
AQSLQLSGGAQ

SEQ ID NO: 493 - protein, 11 aa, synthetic construct
AQDKVPHNVAQ

SEQ ID NO: 494 - protein, 11 aa, synthetic construct
AQGVTHVAGAQ

SEQ ID NO: 495 - protein, 11 aa, synthetic construct
AQIAGMSSYAQ

SEQ ID NO: 496 - protein, 11 aa, synthetic construct
AQLLPSGISAQ

SEQ ID NO: 497 - protein, 11 aa, synthetic construct
AQYDNIARNAQ

SEQ ID NO: 498 - protein, 11 aa, synthetic construct
AQGTSLKVSAQ

SEQ ID NO: 499 - protein, 11 aa, synthetic construct
AQLSRAQAVAQ

SEQ ID NO: 500 - protein, 11 aa, synthetic construct
AQGKASLSGAQ

SEQ ID NO: 501 - protein, 11 aa, synthetic construct
AQYNREASGAQ

SEQ ID NO: 502 - protein, 11 aa, synthetic construct
AQRLDSRPSAQ

SEQ ID NO: 503 - protein, 11 aa, synthetic construct
AQRMRVDGEAQ

SEQ ID NO: 504 - protein, 11 aa, synthetic construct
AQQVTSVTSAQ

SEQ ID NO: 505 - protein, 11 aa, synthetic construct
AQIITGRSEAQ

SEQ ID NO: 506 - protein, 11 aa, synthetic construct
AQLARLTTEAQ

SEQ ID NO: 507 - protein, 11 aa, synthetic construct
AQMTPSLLIAQ

SEQ ID NO: 508 - protein, 11 aa, synthetic construct
AQSNFSVHSAQ

SEQ ID NO: 509 - protein, 11 aa, synthetic construct
AQESSVLQLAQ

SEQ ID NO: 510 - protein, 11 aa, synthetic construct
AQSNYSSRDAQ

SEQ ID NO: 511 - protein, 11 aa, synthetic construct
AQEMSRGTQAQ

SEQ ID NO: 512 - protein, 11 aa, synthetic construct
AQSDQTRILAQ

SEQ ID NO: 513 - protein, 11 aa, synthetic construct
AQSQNVNLGAQ

SEQ ID NO: 514 - protein, 11 aa, synthetic construct
AQTERNLSTAQ

SEQ ID NO: 515 - protein, 11 aa, synthetic construct
AQSTGTLHGAQ

SEQ ID NO: 516 - protein, 11 aa, synthetic construct
AQSMHLRQEAQ

SEQ ID NO: 517 - protein, 11 aa, synthetic construct
AQLMSVRQDAQ

SEQ ID NO: 518 - protein, 11 aa, synthetic construct
AQAERSSFEAQ

SEQ ID NO: 519 - protein, 11 aa, synthetic construct
AQSPSSSRMAQ

SEQ ID NO: 520 - protein, 11 aa, synthetic construct
AQHSTGTTMAQ

SEQ ID NO: 521 - protein, 11 aa, synthetic construct
AQRSTTDASAQ

SEQ ID NO: 522 - protein, 11 aa, synthetic construct
AQARQMSTAAQ

SEQ ID NO: 523 - protein, 11 aa, synthetic construct
AQINASAPFAQ

SEQ ID NO: 524 - protein, 11 aa, synthetic construct
AQREGMPFLAQ

SEQ ID NO: 525 - protein, 11 aa, synthetic construct
AQQPTLISPAQ

SEQ ID NO: 526 - protein, 11 aa, synthetic construct
AQFNDRSNSAQ

SEQ ID NO: 527 - protein, 11 aa, synthetic construct
AQLPSHMASAQ

SEQ ID NO: 528 - protein, 11 aa, synthetic construct
AQMTVQVSSAQ

SEQ ID NO: 529 - protein, 11 aa, synthetic construct
AQMQSGAITAQ

SEQ ID NO: 530 - protein, 11 aa, synthetic construct
AQLITSLHEAQ

SEQ ID NO: 531 - protein, 11 aa, synthetic construct
AQSSASSVTAQ

SEQ ID NO: 532 - protein, 11 aa, synthetic construct
AQTNHVAHGAQ

SEQ ID NO: 533 - protein, 11 aa, synthetic construct
AQIYTSHAVAQ

SEQ ID NO: 534 - protein, 11 aa, synthetic construct
AQSLPGHLGAQ

SEQ ID NO: 535 - protein, 11 aa, synthetic construct
AQNSSDASRAQ

SEQ ID NO: 536 - protein, 11 aa, synthetic construct
AQVTGTYGHAQ

SEQ ID NO: 537 - protein, 11 aa, synthetic construct
AQSGHFPSGAQ

SEQ ID NO: 538 - protein, 11 aa, synthetic construct
AQIAVGSTTAQ

SEQ ID NO: 539 - protein, 11 aa, synthetic construct
AQQASQSKMAQ

SEQ ID NO: 540 - protein, 11 aa, synthetic construct
AQHAPSSPHAQ

SEQ ID NO: 541 - protein, 11 aa, synthetic construct
AQRLLDSGHAQ

SEQ ID NO: 542 - protein, 11 aa, synthetic construct
AQMNTVRPHAQ

SEQ ID NO: 543 - protein, 11 aa, synthetic construct
AQSHHKQGVAQ

SEQ ID NO: 544 - protein, 11 aa, synthetic construct
AQQHNGPSVAQ

SEQ ID NO: 545 - protein, 11 aa, synthetic construct
AQLPPTHTQAQ

SEQ ID NO: 546 - protein, 11 aa, synthetic construct
AQERHKDPVAQ

SEQ ID NO: 547 - protein, 11 aa, synthetic construct
AQLPPTRPEAQ

SEQ ID NO: 548 - protein, 11 aa, synthetic construct
AQSEHKGLTAQ

SEQ ID NO: 549 - protein, 11 aa, synthetic construct
AQMKAILTQAQ

SEQ ID NO: 550 - protein, 11 aa, synthetic construct
AQEYRQSLVAQ

SEQ ID NO: 551 - protein, 11 aa, synthetic construct
AQRSNQESVAQ

SEQ ID NO: 552 - protein, 11 aa, synthetic construct
AQSSFNLVSAQ

SEQ ID NO: 553 - protein, 11 aa, synthetic construct
AQLRVSTGVAQ

SEQ ID NO: 554 - protein, 11 aa, synthetic construct
AQGARTLSTAQ

SEQ ID NO: 555 - protein, 11 aa, synthetic construct
AQELSGSRPAQ

SEQ ID NO: 556 - protein, 11 aa, synthetic construct
AQVSTTVPSAQ

SEQ ID NO: 557 - protein, 11 aa, synthetic construct
AQRTQAFLGAQ

SEQ ID NO: 558 - protein, 11 aa, synthetic construct
AQSVAHEGTAQ

SEQ ID NO: 559 - protein, 11 aa, synthetic construct
AQITKQFQSAQ

SEQ ID NO: 560 - protein, 11 aa, synthetic construct
AQILAKTGSAQ

SEQ ID NO: 561 - protein, 11 aa, synthetic construct
AQNTFASTGAQ

SEQ ID NO: 562 - protein, 11 aa, synthetic construct
AQITPRLHSAQ

SEQ ID NO: 563 - protein, 11 aa, synthetic construct
AQGGAALQPAQ

SEQ ID NO: 564 - protein, 11 aa, synthetic construct
AQSSTRHDGAQ

SEQ ID NO: 565 - protein, 11 aa, synthetic construct
AQLSQRSVLAQ

SEQ ID NO: 566 - protein, 11 aa, synthetic construct
AQSVSFSTGAQ

SEQ ID NO: 567 - protein, 11 aa, synthetic construct
AQLPSTANDAQ

SEQ ID NO: 568 - protein, 11 aa, synthetic construct
AQVGVNLSYAQ

SEQ ID NO: 569 - protein, 11 aa, synthetic construct
AQTSSPGYMAQ

SEQ ID NO: 570 - protein, 11 aa, synthetic construct
AQSLHMSASAQ

SEQ ID NO: 571 - protein, 11 aa, synthetic construct
AQLHSLSVTAQ

SEQ ID NO: 572 - protein, 11 aa, synthetic construct
AQSQSKIPSAQ

SEQ ID NO: 573 - protein, 11 aa, synthetic construct
AQRDARMSDAQ

SEQ ID NO: 574 - protein, 11 aa, synthetic construct
AQDRAHGFTAQ

SEQ ID NO: 575 - protein, 11 aa, synthetic construct
AQSSNSRNPAQ

SEQ ID NO: 576 - protein, 11 aa, synthetic construct
AQPSTGRQPAQ

SEQ ID NO: 577 - protein, 11 aa, synthetic construct
AQDRLAVESAQ

SEQ ID NO: 578 - protein, 11 aa, synthetic construct
AQNLVSHVIAQ

SEQ ID NO: 579 - protein, 11 aa, synthetic construct
AQLTTNLRGAQ

SEQ ID NO: 580 - protein, 11 aa, synthetic construct
AQETHGTSMAQ

SEQ ID NO: 581 - protein, 11 aa, synthetic construct
AQMLKSPLTAQ

SEQ ID NO: 582 - protein, 11 aa, synthetic construct
AQSHLSVSSAQ

SEQ ID NO: 583 - protein, 11 aa, synthetic construct
AQYGAVTLTAQ

SEQ ID NO: 584 - protein, 11 aa, synthetic construct
AQIAHNRGPAQ

SEQ ID NO: 585 - protein, 11 aa, synthetic construct
AQIGTVGSPAQ

SEQ ID NO: 586 - protein, 11 aa, synthetic construct
AQGMLRGDPAQ

SEQ ID NO: 587 - protein, 11 aa, synthetic construct
AQRISGGNEAQ

SEQ ID NO: 588 - protein, 11 aa, synthetic construct
AQMGSSHTNAQ

SEQ ID NO: 589 - protein, 11 aa, synthetic construct
AQDHQKMAQAQ

SEQ ID NO: 590 - protein, 11 aa, synthetic construct
AQQQTSRSLAQ

SEQ ID NO: 591 - protein, 11 aa, synthetic construct
AQLMHLGNSAQ

SEQ ID NO: 592 - protein, 11 aa, synthetic construct
AQMSKLLHDAQ

SEQ ID NO: 593 - protein, 11 aa, synthetic construct
AQLLLGTAEAQ

SEQ ID NO: 594 - protein, 11 aa, synthetic construct
AQYPHQTSSAQ

SEQ ID NO: 595 - protein, 11 aa, synthetic construct
AQLPYAGTSAQ

SEQ ID NO: 596 - protein, 11 aa, synthetic construct
AQITTTSTAAQ

SEQ ID NO: 597 - protein, 11 aa, synthetic construct
AQSTRGQLEAQ

SEQ ID NO: 598 - protein, 11 aa, synthetic construct
AQLTSLGNLAQ

SEQ ID NO: 599 - protein, 11 aa, synthetic construct
AQNSMTLKEAQ

SEQ ID NO: 600 - protein, 11 aa, synthetic construct
AQSLMSAMSAQ

SEQ ID NO: 601 - protein, 11 aa, synthetic construct
AQESSRTDRAQ

SEQ ID NO: 602 - protein, 11 aa, synthetic construct
AQHTSYTPAAQ

SEQ ID NO: 603 - protein, 11 aa, synthetic construct
AQISTTSSVAQ

SEQ ID NO: 604 - protein, 11 aa, synthetic construct
AQSRTASLIAQ

SEQ ID NO: 605 - protein, 11 aa, synthetic construct
AQSISSATGAQ

SEQ ID NO: 606 - protein, 11 aa, synthetic construct
AQSSHMGTMAQ

SEQ ID NO: 607 - protein, 11 aa, synthetic construct
AQLAREPLVAQ

SEQ ID NO: 608 - protein, 11 aa, synthetic construct
AQAIRGTLIAQ

SEQ ID NO: 609 - protein, 11 aa, synthetic construct
AQSAVSHKDAQ

SEQ ID NO: 610 - protein, 11 aa, synthetic construct
AQLGLSTSRAQ

SEQ ID NO: 611 - protein, 11 aa, synthetic construct
AQMNPSSNAAQ

SEQ ID NO: 612 - protein, 11 aa, synthetic construct
AQQRSQVDLAQ

SEQ ID NO: 613 - protein, 11 aa, synthetic construct
AQHEPTRGNAQ

SEQ ID NO: 614 - protein, 11 aa, synthetic construct
AQPMSGSTYAQ

SEQ ID NO: 615 - protein, 11 aa, synthetic construct
AQHTSLPTTAQ

SEQ ID NO: 616 - protein, 11 aa, synthetic construct
AQILQHSLSAQ

SEQ ID NO: 617 - protein, 11 aa, synthetic construct
AQARVMTGEAQ

SEQ ID NO: 618 - protein, 11 aa, synthetic construct
AQLHQRVNPAQ

SEQ ID NO: 619 - protein, 11 aa, synthetic construct
AQVTSGYSLAQ

SEQ ID NO: 620 - protein, 11 aa, synthetic construct
AQLPSAHHFAQ

SEQ ID NO: 621 - protein, 11 aa, synthetic construct
AQHNYTSQVAQ

SEQ ID NO: 622 - protein, 11 aa, synthetic construct
AQERNYPVMAQ

SEQ ID NO: 623 - protein, 11 aa, synthetic construct
AQPQGGSSSAQ

SEQ ID NO: 624 - protein, 11 aa, synthetic construct
AQSDRVFAYAQ

SEQ ID NO: 625 - protein, 11 aa, synthetic construct
AQGVQHIGQAQ

SEQ ID NO: 626 - protein, 11 aa, synthetic construct
AQSLPPSHAAQ

SEQ ID NO: 627 - protein, 11 aa, synthetic construct
AQSSKTIMSAQ

SEQ ID NO: 628 - protein, 11 aa, synthetic construct
AQVRLEIQNAQ

SEQ ID NO: 629 - protein, 11 aa, synthetic construct
AQSLAQNLHAQ

SEQ ID NO: 630 - protein, 11 aa, synthetic construct
AQSSRVSSMAQ

SEQ ID NO: 631 - protein, 11 aa, synthetic construct
AQSSVVRTPAQ

SEQ ID NO: 632 - protein, 11 aa, synthetic construct
AQLPIGTTLAQ

SEQ ID NO: 633 - protein, 11 aa, synthetic construct
AQAYDTVRQAQ

SEQ ID NO: 634 - protein, 11 aa, synthetic construct
AQRQDMVLSAQ

SEQ ID NO: 635 - protein, 11 aa, synthetic construct
AQEKSSGISAQ

SEQ ID NO: 636 - protein, 11 aa, synthetic construct
AQPVGIRPSAQ

SEQ ID NO: 637 - protein, 11 aa, synthetic construct
AQVKTNLQFAQ

SEQ ID NO: 638 - protein, 11 aa, synthetic construct
AQFRSSLSSAQ

SEQ ID NO: 639 - protein, 11 aa, synthetic construct
AQANTGGALAQ

SEQ ID NO: 640 - protein, 11 aa, synthetic construct
AQNGQIKVEAQ

SEQ ID NO: 641 - protein, 11 aa, synthetic construct
AQTTRVGQLAQ

SEQ ID NO: 642 - protein, 11 aa, synthetic construct
AQFVSNHTPAQ

SEQ ID NO: 643 - protein, 11 aa, synthetic construct
AQLQITHGTAQ

SEQ ID NO: 644 - protein, 11 aa, synthetic construct
AQLPNTSRTAQ

SEQ ID NO: 645 - protein, 11 aa, synthetic construct
AQILGLQNKAQ

SEQ ID NO: 646 - protein, 11 aa, synthetic construct
AQYQPQNYAAQ

SEQ ID NO: 647 - protein, 11 aa, synthetic construct
AQHFSGRVEAQ

SEQ ID NO: 648 - protein, 11 aa, synthetic construct
AQAVLSPFVAQ

SEQ ID NO: 649 - protein, 11 aa, synthetic construct
AQTTIIRHSAQ

SEQ ID NO: 650 - protein, 11 aa, synthetic construct
AQVLSSGLMAQ

SEQ ID NO: 651 - protein, 11 aa, synthetic construct
AQLAKSSQSAQ

SEQ ID NO: 652 - protein, 11 aa, synthetic construct
AQIQSALLGAQ

SEQ ID NO: 653 - protein, 11 aa, synthetic construct
AQAKLTHAGAQ

SEQ ID NO: 654 - protein, 11 aa, synthetic construct
AQLTHMKSDAQ

SEQ ID NO: 655 - protein, 11 aa, synthetic construct
AQTTSSSGVAQ

SEQ ID NO: 656 - protein, 11 aa, synthetic construct
AQISVSISSAQ

SEQ ID NO: 657 - protein, 11 aa, synthetic construct
AQSASQMSGAQ

SEQ ID NO: 658 - protein, 11 aa, synthetic construct
AQNSVHSTGAQ

SEQ ID NO: 659 - protein, 11 aa, synthetic construct
APARSTSLTAQ

SEQ ID NO: 660 - protein, 11 aa, synthetic construct
AQNQADSRLAQ

SEQ ID NO: 661 - protein, 11 aa, synthetic construct
AQNYDGRLGAQ

SEQ ID NO: 662 - protein, 11 aa, synthetic construct
AQLTQRSGAAQ

SEQ ID NO: 663 - protein, 11 aa, synthetic construct
AQSMPSKTAAQ

SEQ ID NO: 664 - protein, 11 aa, synthetic construct
AQSNRVGPPAQ

SEQ ID NO: 665 - protein, 11 aa, synthetic construct
AQLTRNVAPAQ

SEQ ID NO: 666 - protein, 11 aa, synthetic construct
AQSQTKETLAQ

SEQ ID NO: 667 - protein, 11 aa, synthetic construct
AQETQRGSSAQ

SEQ ID NO: 668 - protein, 11 aa, synthetic construct
AQLVPSRVVAQ

SEQ ID NO: 669 - protein, 11 aa, synthetic construct
AQMDRSKSAAQ

SEQ ID NO: 670 - protein, 11 aa, synthetic construct
AQVTTGVSQAQ

SEQ ID NO: 671 - protein, 11 aa, synthetic construct
AQQKLQNNMAQ

SEQ ID NO: 672 - protein, 11 aa, synthetic construct
AQASVQATPAQ

SEQ ID NO: 673 - protein, 11 aa, synthetic construct
AQHTKVHSGAQ

SEQ ID NO: 674 - protein, 11 aa, synthetic construct
AQSLPSNLIAQ

SEQ ID NO: 675 - protein, 11 aa, synthetic construct
AQLGGGFSEAQ

SEQ ID NO: 676 - protein, 11 aa, synthetic construct
AQNGVRVLSAQ

SEQ ID NO: 677 - protein, 11 aa, synthetic construct
AQMSKPHEAAQ

SEQ ID NO: 678 - protein, 11 aa, synthetic construct
AQTSRLSTVAQ

SEQ ID NO: 679 - protein, 11 aa, synthetic construct
AQSNLSPKEAQ

SEQ ID NO: 680 - protein, 11 aa, synthetic construct
AQLPQHNHSAQ

SEQ ID NO: 681 - protein, 11 aa, synthetic construct
AQGSHVLAPAQ

SEQ ID NO: 682 - protein, 11 aa, synthetic construct
AQRERTPVVAQ

SEQ ID NO: 683 - protein, 11 aa, synthetic construct
AQSSAAGQIAQ

SEQ ID NO: 684 - protein, 11 aa, synthetic construct
AQYTYSTAVAQ

SEQ ID NO: 685 - protein, 11 aa, synthetic construct
AQIHAGIANAQ

SEQ ID NO: 686 - protein, 11 aa, synthetic construct
AQTVARVSVAQ

SEQ ID NO: 687 - protein, 11 aa, synthetic construct
AQRDTPGINAQ

SEQ ID NO: 688 - protein, 11 aa, synthetic construct
AQTAKSTHLAQ

SEQ ID NO: 689 - protein, 11 aa, synthetic construct
AQHREPSLTAQ

SEQ ID NO: 690 - protein, 11 aa, synthetic construct
AQSRAAGNVAQ

SEQ ID NO: 691 - protein, 11 aa, synthetic construct
AQPATSRFDAQ

SEQ ID NO: 692 - protein, 11 aa, synthetic construct
AQGQSSGVAAQ

SEQ ID NO: 693 - protein, 11 aa, synthetic construct
AQDTLSSAGAQ

SEQ ID NO: 694 - protein, 11 aa, synthetic construct
AQALNRSPFAQ

SEQ ID NO: 695 - protein, 11 aa, synthetic construct
AQIMAAVTTAQ

SEQ ID NO: 696 - protein, 11 aa, synthetic construct
AQAHLANSPAQ

SEQ ID NO: 697 - protein, 11 aa, synthetic construct
AQGTSSTPTAQ

SEQ ID NO: 698 - protein, 11 aa, synthetic construct
AQIGGNLLKAQ

SEQ ID NO: 699 - protein, 11 aa, synthetic construct
AQQESQIPNAQ

SEQ ID NO: 700 - protein, 11 aa, synthetic construct
AQIHPPSSSAQ

SEQ ID NO: 701 - protein, 11 aa, synthetic construct
AQTSSQTLRAQ

SEQ ID NO: 702 - protein, 11 aa, synthetic construct
AQETTGHSGAQ

SEQ ID NO: 703 - protein, 11 aa, synthetic construct
AQLLSNRVGAQ

SEQ ID NO: 704 - protein, 11 aa, synthetic construct
AQVQRGDQLAQ

SEQ ID NO: 705 - protein, 11 aa, synthetic construct
AQPTTSVSAAQ

SEQ ID NO: 706 - protein, 11 aa, synthetic construct
AQMFATVASAQ

SEQ ID NO: 707 - protein, 11 aa, synthetic construct
AQVRPSMSAAQ

SEQ ID NO: 708 - protein, 11 aa, synthetic construct
AQLSGSMQPAQ

SEQ ID NO: 709 - protein, 11 aa, synthetic construct
AQHSNQGSLAQ

SEQ ID NO: 710 - protein, 11 aa, synthetic construct
AQRIGVHTSAQ

SEQ ID NO: 711 - protein, 11 aa, synthetic construct
AQPLRHEGGAQ

SEQ ID NO: 712 - protein, 11 aa, synthetic construct
AQTTKPGVIAQ

SEQ ID NO: 713 - protein, 11 aa, synthetic construct
AQIPSKVQMAQ

SEQ ID NO: 714 - protein, 11 aa, synthetic construct
AQRISQTTLAQ

SEQ ID NO: 715 - protein, 11 aa, synthetic construct
AQRTPVNSIAQ

SEQ ID NO: 716 - protein, 11 aa, synthetic construct
AQLTQVRTVAQ

SEQ ID NO: 717 - protein, 11 aa, synthetic construct
AQQLVGSGVAQ

SEQ ID NO: 718 - protein, 11 aa, synthetic construct
AQTSAITSSAQ

SEQ ID NO: 719 - protein, 11 aa, synthetic construct
AQTGVAPTPAQ

SEQ ID NO: 720 - protein, 11 aa, synthetic construct
AQANLVPHGAQ

SEQ ID NO: 721 - protein, 11 aa, synthetic construct
AQLARQPHEAQ

SEQ ID NO: 722 - protein, 11 aa, synthetic construct
APRVMQSSVAQ

SEQ ID NO: 723 - protein, 11 aa, synthetic construct
AQYTPSGFLAQ

SEQ ID NO: 724 - protein, 11 aa, synthetic construct
AQGAIKENLAQ

SEQ ID NO: 725 - protein, 11 aa, synthetic construct
AQTQSNFSGAQ

SEQ ID NO: 726 - protein, 11 aa, synthetic construct
AQIGSQMQNAQ

SEQ ID NO: 727 - protein, 11 aa, synthetic construct
AQSAHRLTAAQ

SEQ ID NO: 728 - protein, 11 aa, synthetic construct
AQNDARSPMAQ

SEQ ID NO: 729 - protein, 11 aa, synthetic construct
AQNGLTTISAQ

SEQ ID NO: 730 - protein, 11 aa, synthetic construct
AQSVPPSAYAQ

SEQ ID NO: 731 - protein, 11 aa, synthetic construct
AQSEPKTNSAQ

SEQ ID NO: 732 - protein, 11 aa, synthetic construct
AQSGNGAVMAQ

SEQ ID NO: 733 - protein, 11 aa, synthetic construct
AQGQDSHHSAQ

SEQ ID NO: 734 - protein, 11 aa, synthetic construct
AQTMSGSQTAQ

SEQ ID NO: 735 - protein, 11 aa, synthetic construct
AQILQAGGQAQ

SEQ ID NO: 736 - protein, 11 aa, synthetic construct
AQRNQASIFAQ

SEQ ID NO: 737 - protein, 11 aa, synthetic construct
AQKVNIIQGAQ

SEQ ID NO: 738 - protein, 11 aa, synthetic construct
AQVLGGAHLAQ

SEQ ID NO: 739 - protein, 11 aa, synthetic construct
AQSTHGTMVAQ

SEQ ID NO: 740 - protein, 11 aa, synthetic construct
AQIMTSPMSAQ

SEQ ID NO: 741 - protein, 11 aa, synthetic construct
AQMRVSSTGAQ

SEQ ID NO: 742 - protein, 11 aa, synthetic construct
AQMSLRSDVAQ

SEQ ID NO: 743 - protein, 11 aa, synthetic construct
AQVSSMTGHAQ

SEQ ID NO: 744 - protein, 11 aa, synthetic construct
AQFNVVGDHAQ

SEQ ID NO: 745 - protein, 11 aa, synthetic construct
AQSYLSRTEAQ

SEQ ID NO: 746 - protein, 11 aa, synthetic construct
AQTVNVTLAAQ

SEQ ID NO: 747 - protein, 11 aa, synthetic construct
AQTSTSSLRAQ

SEQ ID NO: 748 - protein, 11 aa, synthetic construct
AQALSHGYSAQ

SEQ ID NO: 749 - protein, 11 aa, synthetic construct
AQMIGTASGAQ

SEQ ID NO: 750 - protein, 11 aa, synthetic construct
AQPLSTSRLAQ

SEQ ID NO: 751 - protein, 11 aa, synthetic construct
AQNTISTSVAQ

SEQ ID NO: 752 - protein, 11 aa, synthetic construct
AQFLKGGLPAQ

SEQ ID NO: 753 - protein, 11 aa, synthetic construct
AQLVIRSSVAQ

SEQ ID NO: 754 - protein, 11 aa, synthetic construct
AQSSGSQNLAQ

SEQ ID NO: 755 - protein, 11 aa, synthetic construct
AQYAQFTGQAQ

SEQ ID NO: 756 - protein, 11 aa, synthetic construct
AQGMQSLTGAQ

SEQ ID NO: 757 - protein, 11 aa, synthetic construct
AQKSSQVLVAQ

SEQ ID NO: 758 - protein, 11 aa, synthetic construct
AQESRPSSFAQ

SEQ ID NO: 759 - protein, 11 aa, synthetic construct
AQLQLFTTEAQ

SEQ ID NO: 760 - protein, 11 aa, synthetic construct
AQRATTMDSAQ

SEQ ID NO: 761 - protein, 11 aa, synthetic construct
AQVSRAVTFAQ

SEQ ID NO: 762 - protein, 11 aa, synthetic construct
AQQLNKAKEAQ

SEQ ID NO: 763 - protein, 11 aa, synthetic construct
AQMRAVVSTAQ

SEQ ID NO: 764 - protein, 11 aa, synthetic construct
AQPLSRETIAQ

SEQ ID NO: 765 - protein, 11 aa, synthetic construct
AQKVHMIGTAQ

SEQ ID NO: 766 - protein, 11 aa, synthetic construct
AQLISGTSLAQ

SEQ ID NO: 767 - protein, 11 aa, synthetic construct
AQVSHPVMRAQ

SEQ ID NO: 768 - protein, 11 aa, synthetic construct
AQGISIGSDAQ

SEQ ID NO: 769 - protein, 11 aa, synthetic construct
AQKNTSQGWAQ

SEQ ID NO: 770 - protein, 11 aa, synthetic construct
AQAGAPTASAQ

SEQ ID NO: 771 - protein, 11 aa, synthetic construct
AQSGKMGETAQ

SEQ ID NO: 772 - protein, 11 aa, synthetic construct
AQPHNNIGPAQ

SEQ ID NO: 773 - protein, 11 aa, synthetic construct
AQMSRGEVLAQ

SEQ ID NO: 774 - protein, 11 aa, synthetic construct
AQLSTAIRGAQ

SEQ ID NO: 775 - protein, 11 aa, synthetic construct
AQVGLSHTPAQ

SEQ ID NO: 776 - protein, 11 aa, synthetic construct
AQHSLSPTPAQ

SEQ ID NO: 777 - protein, 11 aa, synthetic construct
AQLSGLVHMAQ

SEQ ID NO: 778 - protein, 11 aa, synthetic construct
AQSRPQSMVAQ

SEQ ID NO: 779 - protein, 11 aa, synthetic construct
AQSLSGSAIAQ

SEQ ID NO: 780 - protein, 11 aa, synthetic construct
AQISSHHLSAQ

SEQ ID NO: 781 - protein, 11 aa, synthetic construct
AQLSVVTHLAQ

SEQ ID NO: 782 - protein, 11 aa, synthetic construct
AQSLHSAFHAQ

SEQ ID NO: 783 - protein, 11 aa, synthetic construct
AQQLSSSIDAQ

SEQ ID NO: 784 - protein, 11 aa, synthetic construct
AQSGLSLHLAQ

SEQ ID NO: 785 - protein, 11 aa, synthetic construct
AQSSVRYSEAQ

SEQ ID NO: 786 - protein, 11 aa, synthetic construct
AQITSRSTTAQ

SEQ ID NO: 787 - protein, 11 aa, synthetic construct
AQHARGTSEAQ

SEQ ID NO: 788 - protein, 11 aa, synthetic construct
AQMVARETIAQ

SEQ ID NO: 789 - protein, 11 aa, synthetic construct
AQFKSQSSSAQ

SEQ ID NO: 790 - protein, 11 aa, synthetic construct
AQYGTTYQLAQ

SEQ ID NO: 791 - protein, 11 aa, synthetic construct
AQLLTSSLSAQ

SEQ ID NO: 792 - protein, 11 aa, synthetic construct
AQSMAYSNEAQ

SEQ ID NO: 793 - protein, 11 aa, synthetic construct
AQVSNLIGVAQ

SEQ ID NO: 794 - protein, 11 aa, synthetic construct
AQSPKLHGDAQ

SEQ ID NO: 795 - protein, 11 aa, synthetic construct
AQLSVLVHNAQ

SEQ ID NO: 796 - protein, 11 aa, synthetic construct
AQSLTRTPVAQ

SEQ ID NO: 797 - protein, 11 aa, synthetic construct
AQIGNLSAVAQ

SEQ ID NO: 798 - protein, 11 aa, synthetic construct
AQINHLKSPAQ

SEQ ID NO: 799 - protein, 11 aa, synthetic construct
AQVLLPSVSAQ

SEQ ID NO: 800 - protein, 11 aa, synthetic construct
AQGVRTPQVAQ

SEQ ID NO: 801 - protein, 11 aa, synthetic construct
AQMVNSRDGAQ

SEQ ID NO: 802 - protein, 11 aa, synthetic construct
AQVVLTGQIAQ

SEQ ID NO: 803 - protein, 11 aa, synthetic construct
AQKVMNAVDAQ

SEQ ID NO: 804 - protein, 11 aa, synthetic construct
AQTMAPDRFAQ

SEQ ID NO: 805 - protein, 11 aa, synthetic construct
AQSFDRTQHAQ

SEQ ID NO: 806 - protein, 11 aa, synthetic construct
AQLSGSPKSAQ

SEQ ID NO: 807 - protein, 11 aa, synthetic construct
AQLGIQSTIAQ

SEQ ID NO: 808 - protein, 11 aa, synthetic construct
AQSNMASTPAQ

SEQ ID NO: 809 - protein, 11 aa, synthetic construct
AQSTSYDLAAQ

SEQ ID NO: 810 - protein, 11 aa, synthetic construct
AQNLVHPTGAQ

SEQ ID NO: 811 - protein, 11 aa, synthetic construct
AQLNHNTPTAQ

SEQ ID NO: 812 - protein, 11 aa, synthetic construct
AQLLPMGTAAQ

SEQ ID NO: 813 - protein, 11 aa, synthetic construct
AQTQGSSRFAQ

SEQ ID NO: 814 - protein, 11 aa, synthetic construct
AQVIRMEVEAQ

SEQ ID NO: 815 - protein, 11 aa, synthetic construct
AQQPSHTTGAQ

SEQ ID NO: 816 - protein, 11 aa, synthetic construct
AQMVQRNQLAQ

SEQ ID NO: 817 - protein, 11 aa, synthetic construct
AQVTASVLSAQ

SEQ ID NO: 818 - protein, 11 aa, synthetic construct
AQVQSLARVAQ

SEQ ID NO: 819 - protein, 11 aa, synthetic construct
AQPAHSISGAQ

SEQ ID NO: 820 - protein, 11 aa, synthetic construct
AQRADGYSYAQ

SEQ ID NO: 821 - protein, 11 aa, synthetic construct
AQMTTNLIAAQ

SEQ ID NO: 822 - protein, 11 aa, synthetic construct
AQWTNKAPSAQ

SEQ ID NO: 823 - protein, 11 aa, synthetic construct
AQHTSTSTVAQ

SEQ ID NO: 824 - protein, 11 aa, synthetic construct
AQNQHSMTSAQ

SEQ ID NO: 825 - protein, 11 aa, synthetic construct
AQTNTATHMAQ

SEQ ID NO: 826 - protein, 11 aa, synthetic construct
AQLVMSLHSAQ

SEQ ID NO: 827 - protein, 11 aa, synthetic construct
AQSGHGSMNAQ

SEQ ID NO: 828 - protein, 11 aa, synthetic construct
AQLQKSLVVAQ

SEQ ID NO: 829 - protein, 11 aa, synthetic construct
AQSLTSISAAQ

SEQ ID NO: 830 - protein, 11 aa, synthetic construct
AQYIVGASNAQ

SEQ ID NO: 831 - protein, 11 aa, synthetic construct
AQLSGRYVAAQ

SEQ ID NO: 832 - protein, 11 aa, synthetic construct
AQEKSYEGSAQ

SEQ ID NO: 833 - protein, 11 aa, synthetic construct
AQLLGRGQSAQ

SEQ ID NO: 834 - protein, 11 aa, synthetic construct
AQLTHSGISAQ

SEQ ID NO: 835 - protein, 11 aa, synthetic construct
AQLGQERLSAQ

SEQ ID NO: 836 - protein, 11 aa, synthetic construct
AQRVVQGGLAQ

SEQ ID NO: 837 - protein, 11 aa, synthetic construct
AQQISSGYLAQ

SEQ ID NO: 838 - protein, 11 aa, synthetic construct
AQGNSYTVIAQ

SEQ ID NO: 839 - protein, 11 aa, synthetic construct
AQANSLSSAAQ

SEQ ID NO: 840 - protein, 11 aa, synthetic construct
AQTHNVEARAQ

SEQ ID NO: 841 - protein, 11 aa, synthetic construct
AQGLSRNEEAQ

SEQ ID NO: 842 - protein, 11 aa, synthetic construct
AQSPKAVLPAQ

SEQ ID NO: 843 - protein, 11 aa, synthetic construct
AQISQSLVVAQ

SEQ ID NO: 844 - protein, 11 aa, synthetic construct
AQERRDLQMAQ

SEQ ID NO: 845 - protein, 11 aa, synthetic construct
AQLSNSVKSVQ

SEQ ID NO: 846 - protein, 11 aa, synthetic construct
AQVLSSSMIAQ

SEQ ID NO: 847 - protein, 11 aa, synthetic construct
AQSRSHSVEAQ

SEQ ID NO: 848 - protein, 11 aa, synthetic construct
AQLITVSNLAQ

SEQ ID NO: 849 - protein, 11 aa, synthetic construct
AQTSINATHAQ

SEQ ID NO: 850 - protein, 11 aa, synthetic construct
AQHTATDYRAQ

SEQ ID NO: 851 - protein, 11 aa, synthetic construct
AQYNQFTIAAQ

SEQ ID NO: 852 - protein, 11 aa, synthetic construct
AQVRYEEPTAQ

SEQ ID NO: 853 - protein, 11 aa, synthetic construct
AQSHNLAISAQ

SEQ ID NO: 854 - protein, 11 aa, synthetic construct
AQLTTASVQAQ

SEQ ID NO: 855 - protein, 11 aa, synthetic construct
AQGTNPKMGAQ

SEQ ID NO: 856 - protein, 11 aa, synthetic construct
AQKTASTVLAQ

SEQ ID NO: 857 - protein, 11 aa, synthetic construct
AQVSRTNVNAQ

SEQ ID NO: 858 - protein, 11 aa, synthetic construct
AQFNPQLAHAQ

SEQ ID NO: 859 - protein, 11 aa, synthetic construct
AQHTYGTTGAQ

SEQ ID NO: 860 - protein, 11 aa, synthetic construct
AQSVTAIQGAQ

SEQ ID NO: 861 - protein, 11 aa, synthetic construct
AQKVSISSPAQ

SEQ ID NO: 862 - protein, 11 aa, synthetic construct
AQRQSATQWAQ

SEQ ID NO: 863 - protein, 11 aa, synthetic construct
AQFALTSQSAQ

SEQ ID NO: 864 - protein, 11 aa, synthetic construct
AQMDSHLIRAQ

SEQ ID NO: 865 - protein, 11 aa, synthetic construct
AQSGHPYTLAQ

SEQ ID NO: 866 - protein, 11 aa, synthetic construct
AQLHLSQSPAQ

SEQ ID NO: 867 - protein, 11 aa, synthetic construct
SQNLRDKEMAQ

SEQ ID NO: 868 - protein, 11 aa, synthetic construct
AQLTGVSGVAQ

SEQ ID NO: 869 - protein, 11 aa, synthetic construct
AQNLSQSPSAQ

SEQ ID NO: 870 - protein, 11 aa, synthetic construct
AQLSREMHLAQ

SEQ ID NO: 871 - protein, 11 aa, synthetic construct
AQINASLGMAQ

SEQ ID NO: 872 - protein, 11 aa, synthetic construct
AQNLVQHQFAQ

SEQ ID NO: 873 - protein, 11 aa, synthetic construct
AQVALRLSAAQ

SEQ ID NO: 874 - protein, 11 aa, synthetic construct
AQFSSSGGSAQ

SEQ ID NO: 875 - protein, 11 aa, synthetic construct
AQYGSYSTIAQ

SEQ ID NO: 876 - protein, 11 aa, synthetic construct
AQLSSMSNQAQ

SEQ ID NO: 877 - protein, 11 aa, synthetic construct
AQLQVQRSMAQ

SEQ ID NO: 878 - protein, 11 aa, synthetic construct
AQHTRLATGAQ

SEQ ID NO: 879 - protein, 11 aa, synthetic construct
AQENRQAPSAQ

SEQ ID NO: 880 - protein, 11 aa, synthetic construct
AQTRSGIIVAQ

SEQ ID NO: 881 - protein, 11 aa, synthetic construct
AQSIPSSMFAQ

SEQ ID NO: 882 - protein, 11 aa, synthetic construct
AQDAGTSPAAQ

SEQ ID NO: 883 - protein, 11 aa, synthetic construct
AQPLGSPISAQ

SEQ ID NO: 884 - protein, 11 aa, synthetic construct
AQKLNVSTGAQ

SEQ ID NO: 885 - protein, 11 aa, synthetic construct
AQSLSTSMMAQ

SEQ ID NO: 886 - protein, 11 aa, synthetic construct
AQQVSTRQSAQ

SEQ ID NO: 887 - protein, 11 aa, synthetic construct
AQQDYGAHGAQ

SEQ ID NO: 888 - protein, 11 aa, synthetic construct
AQQSPGYRGAQ

SEQ ID NO: 889 - protein, 11 aa, synthetic construct
AQHDNPRDTAQ

SEQ ID NO: 890 - protein, 11 aa, synthetic construct
AQGLIASNHAQ

SEQ ID NO: 891 - protein, 11 aa, synthetic construct
AQTLHHLPMAQ

SEQ ID NO: 892 - protein, 11 aa, synthetic construct
AQYMKTYPDAQ

SEQ ID NO: 893 - protein, 11 aa, synthetic construct
AQKLPPSNVAQ

SEQ ID NO: 894 - protein, 11 aa, synthetic construct
AQYIHSAGPAQ

SEQ ID NO: 895 - protein, 11 aa, synthetic construct
AQTALNRMGAQ

SEQ ID NO: 896 - protein, 11 aa, synthetic construct
AQVRPTLTSAQ

SEQ ID NO: 897 - protein, 11 aa, synthetic construct
AQGMFGRGPAQ

SEQ ID NO: 898 - protein, 11 aa, synthetic construct
AQSRSSEGSAQ

SEQ ID NO: 899 - protein, 11 aa, synthetic construct
AQQLVSSSVAQ

SEQ ID NO: 900 - protein, 11 aa, synthetic construct
AQVPKYSSGAQ

SEQ ID NO: 901 - protein, 11 aa, synthetic construct
AQIMGSSQIAQ

SEQ ID NO: 902 - protein, 11 aa, synthetic construct
AQTTLQTSIAQ

SEQ ID NO: 903 - protein, 11 aa, synthetic construct
AQVHGSLISAQ

SEQ ID NO: 904 - protein, 11 aa, synthetic construct
AQHVGNGVLAQ

SEQ ID NO: 905 - protein, 11 aa, synthetic construct
AQDLGNLKVAQ

SEQ ID NO: 906 - protein, 11 aa, synthetic construct
AQRISNAGYAQ

SEQ ID NO: 907 - protein, 11 aa, synthetic construct
AQNPPSLFTAQ

SEQ ID NO: 908 - protein, 11 aa, synthetic construct
AQSNTYSIVAQ

SEQ ID NO: 909 - protein, 11 aa, synthetic construct
AQSTRQISTAQ

SEQ ID NO: 910 - protein, 11 aa, synthetic construct
AQNPKASLPAQ

SEQ ID NO: 911 - protein, 11 aa, synthetic construct
AQLSDSRYEAQ

SEQ ID NO: 912 - protein, 11 aa, synthetic construct
AQSANSIGSAQ

SEQ ID NO: 913 - protein, 11 aa, synthetic construct
AQDLTPHKDAQ

SEQ ID NO: 914 - protein, 11 aa, synthetic construct
AQRVHLVSGAQ

SEQ ID NO: 915 - protein, 11 aa, synthetic construct
AQYSQFAQSAQ

SEQ ID NO: 916 - protein, 11 aa, synthetic construct
AQVIQQSVVAQ

SEQ ID NO: 917 - protein, 11 aa, synthetic construct
AQSYADNQRAQ

SEQ ID NO: 918 - protein, 11 aa, synthetic construct
AQATLASHFAQ

SEQ ID NO: 919 - protein, 11 aa, synthetic construct
AQFQSFSQTAQ

SEQ ID NO: 920 - protein, 11 aa, synthetic construct
AQHFHSHTGAQ

SEQ ID NO: 921 - protein, 11 aa, synthetic construct
AQHNMHGKDAQ

SEQ ID NO: 922 - protein, 11 aa, synthetic construct
AQLSRIATSAQ

SEQ ID NO: 923 - protein, 11 aa, synthetic construct
AQTYRSEDIAQ

SEQ ID NO: 924 - protein, 11 aa, synthetic construct
AQLASDPQNAQ

SEQ ID NO: 925 - protein, 11 aa, synthetic construct
AQVETWSAKAQ

SEQ ID NO: 926 - protein, 11 aa, synthetic construct
AQAFPSGHLAQ

SEQ ID NO: 927 - protein, 11 aa, synthetic construct
AQSRESPPSAQ

SEQ ID NO: 928 - protein, 11 aa, synthetic construct
AQLMHGVGSAQ

SEQ ID NO: 929 - protein, 11 aa, synthetic construct
AQASHSTSSAQ

SEQ ID NO: 930 - protein, 11 aa, synthetic construct
AQPHATTVAAQ

SEQ ID NO: 931 - protein, 11 aa, synthetic construct
AQKQGLLPMAQ

SEQ ID NO: 932 - protein, 11 aa, synthetic construct
AQSSISGIPAQ

SEQ ID NO: 933 - protein, 11 aa, synthetic construct
AQLSSLRNVAQ

SEQ ID NO: 934 - protein, 11 aa, synthetic construct
AQVSAHKMDAQ

SEQ ID NO: 935 - protein, 11 aa, synthetic construct
AQTSGTKVFAQ

SEQ ID NO: 936 - protein, 11 aa, synthetic construct
AQRVNYEQSAQ

SEQ ID NO: 937 - protein, 11 aa, synthetic construct
AQNHNAYLPAQ

SEQ ID NO: 938 - protein, 11 aa, synthetic construct
AQHLSTLRSAQ

SEQ ID NO: 939 - protein, 11 aa, synthetic construct
AQTHSLLHSAQ

SEQ ID NO: 940 - protein, 11 aa, synthetic construct
AQGSASPVTAQ

SEQ ID NO: 941 - protein, 11 aa, synthetic construct
AQGIGGIYLAQ

SEQ ID NO: 942 - protein, 11 aa, synthetic construct
AQSASMQTRAQ

SEQ ID NO: 943 - protein, 11 aa, synthetic construct
AQLRADTIRAQ

SEQ ID NO: 944 - protein, 11 aa, synthetic construct
AQLPSVQSIAQ

SEQ ID NO: 945 - protein, 11 aa, synthetic construct
AQYDNSNPAAQ

SEQ ID NO: 946 - protein, 11 aa, synthetic construct
AQNISSGMRAQ

SEQ ID NO: 947 - protein, 11 aa, synthetic construct
AQTQSSKNDAQ

SEQ ID NO: 948 - protein, 11 aa, synthetic construct
AQVSVSRTSAQ

SEQ ID NO: 949 - protein, 11 aa, synthetic construct
AQFMTLDKMAQ

SEQ ID NO: 950 - protein, 11 aa, synthetic construct
AQEKGNLAGAQ

SEQ ID NO: 951 - protein, 11 aa, synthetic construct
AQTIHTGDMAQ

SEQ ID NO: 952 - protein, 11 aa, synthetic construct
AQSVAKDITAQ

SEQ ID NO: 953 - protein, 11 aa, synthetic construct
AQLLTKSYPAQ

SEQ ID NO: 954 - protein, 11 aa, synthetic construct
AQPKHTMSDAQ

SEQ ID NO: 955 - protein, 11 aa, synthetic construct
AQKAASSTPAQ

SEQ ID NO: 956 - protein, 11 aa, synthetic construct
AQHQRSDLNAQ

SEQ ID NO: 957 - protein, 11 aa, synthetic construct
AQVSSLTAIAQ

SEQ ID NO: 958 - protein, 11 aa, synthetic construct
AQGGRSSPDAQ

SEQ ID NO: 959 - protein, 11 aa, synthetic construct
AQAVMRDSSAQ

SEQ ID NO: 960 - protein, 11 aa, synthetic construct
AQIQPSSKLAQ

SEQ ID NO: 961 - protein, 11 aa, synthetic construct
AQSISATYLAQ

SEQ ID NO: 962 - protein, 11 aa, synthetic construct
AQGVSNTNTAQ

SEQ ID NO: 963 - protein, 11 aa, synthetic construct
AQSVYPAHSAQ

SEQ ID NO: 964 - protein, 11 aa, synthetic construct
AQKGGETSSAQ

SEQ ID NO: 965 - protein, 11 aa, synthetic construct
AQFVTTSEVAQ

SEQ ID NO: 966 - protein, 11 aa, synthetic construct
AQLPVNIGSAQ

SEQ ID NO: 967 - protein, 11 aa, synthetic construct
AQRDIATATAQ

SEQ ID NO: 968 - protein, 11 aa, synthetic construct
AQVGGSTYPAQ

SEQ ID NO: 969 - protein, 11 aa, synthetic construct
AQTSMPRMEAQ

SEQ ID NO: 970 - protein, 11 aa, synthetic construct
AQNLLNPGSAQ

SEQ ID NO: 971 - protein, 11 aa, synthetic construct
AQLDQLKTEAQ

SEQ ID NO: 972 - protein, 11 aa, synthetic construct
AQASEAGMRAQ

SEQ ID NO: 973 - protein, 11 aa, synthetic construct
AQVNFTSNLAQ

SEQ ID NO: 974 - protein, 11 aa, synthetic construct
AQIIHHHTSAQ

SEQ ID NO: 975 - protein, 11 aa, synthetic construct
AQPNPLNASAQ

SEQ ID NO: 976 - protein, 11 aa, synthetic construct
AQDSLPTGRAQ

SEQ ID NO: 977 - protein, 11 aa, synthetic construct
AQLQLLERTAQ

SEQ ID NO: 978 - protein, 11 aa, synthetic construct
AQFGVHGGPAQ

SEQ ID NO: 979 - protein, 11 aa, synthetic construct
AQTVVNLSGAQ

SEQ ID NO: 980 - protein, 11 aa, synthetic construct
AQGHHGKLDAQ

SEQ ID NO: 981 - protein, 11 aa, synthetic construct
AQLPLSVKHAQ

SEQ ID NO: 982 - protein, 11 aa, synthetic construct
AQNRLDTDRAQ

SEQ ID NO: 983 - protein, 11 aa, synthetic construct
AQNRIMNVSAQ

SEQ ID NO: 984 - protein, 11 aa, synthetic construct
AQTTQFVHPAQ

SEQ ID NO: 985 - protein, 11 aa, synthetic construct
AQHDRASSVAQ

SEQ ID NO: 986 - protein, 11 aa, synthetic construct
AQQIPVQLHAQ

SEQ ID NO: 987 - protein, 11 aa, synthetic construct
AQQVHITNAAQ

SEQ ID NO: 988 - protein, 11 aa, synthetic construct
AQASMKHEMAQ

SEQ ID NO: 989 - protein, 11 aa, synthetic construct
AQTITPPRVAQ

SEQ ID NO: 990 - protein, 11 aa, synthetic construct
AQLGQSNSFAQ

SEQ ID NO: 991 - protein, 11 aa, synthetic construct
AQGANMQVGAQ

SEQ ID NO: 992 - protein, 11 aa, synthetic construct
AQLYSSYNAAQ

SEQ ID NO: 993 - protein, 11 aa, synthetic construct
AQIKSVVLTAQ

SEQ ID NO: 994 - protein, 11 aa, synthetic construct
AQLSNVEMSAQ

SEQ ID NO: 995 - protein, 11 aa, synthetic construct
AQYDSVLTKAQ

SEQ ID NO: 996 - protein, 11 aa, synthetic construct
AQNIAAHSHAQ

SEQ ID NO: 997 - protein, 11 aa, synthetic construct
AQTIGLHQVAQ

SEQ ID NO: 998 - protein, 11 aa, synthetic construct
AQLAHMGTQAQ

SEQ ID NO: 999 - protein, 11 aa, synthetic construct
AQTLSFSGAAQ

SEQ ID NO: 1000 - protein, 11 aa, synthetic construct
AQNFPPSTDAQ

SEQ ID NO: 1001 - protein, 11 aa, synthetic construct
AQLPQAGVYAQ

SEQ ID NO: 1002 - protein, 11 aa, synthetic construct
AQSIFLTGTAQ

SEQ ID NO: 1003 - protein, 11 aa, synthetic construct
AQLSQSFKSAQ

SEQ ID NO: 1004 - protein, 11 aa, synthetic construct
AQESVRSSAAQ

SEQ ID NO: 1005 - protein, 11 aa, synthetic construct
AQGSSGMTLAQ

SEQ ID NO: 1006 - protein, 11 aa, synthetic construct
AQLTTVSGNAQ

SEQ ID NO: 1007 - protein, 11 aa, synthetic construct
AQNLKYHAVAQ

SEQ ID NO: 1008 - protein, 11 aa, synthetic construct
AQNALVRNSAQ

SEQ ID NO: 1009 - protein, 11 aa, synthetic construct
AQHSSYHQAAQ

SEQ ID NO: 1010 - protein, 11 aa, synthetic construct
AQKQNHSVSAQ

SEQ ID NO: 1011 - protein, 11 aa, synthetic construct
AQARISTGLAQ

SEQ ID NO: 1012 - protein, 11 aa, synthetic construct
AQNTHKQLGAQ

SEQ ID NO: 1013 - protein, 11 aa, synthetic construct
AQVVSVTSSAQ

SEQ ID NO: 1014 - protein, 11 aa, synthetic construct
AQNINSSSNAQ

SEQ ID NO: 1015 - protein, 11 aa, synthetic construct
AQSLLQRVPAQ

SEQ ID NO: 1016 - protein, 11 aa, synthetic construct
AQIQNTGIAAQ

SEQ ID NO: 1017 - protein, 11 aa, synthetic construct
AQTMSGAAYAQ

SEQ ID NO: 1018 - protein, 11 aa, synthetic construct
AQHIPSSPSAQ

SEQ ID NO: 1019 - protein, 11 aa, synthetic construct
AQYHQVPMQAQ

SEQ ID NO: 1020 - protein, 11 aa, synthetic construct
AQGVTMLVSAQ

SEQ ID NO: 1021 - protein, 11 aa, synthetic construct
AQGLGGPLSAQ

SEQ ID NO: 1022 - protein, 11 aa, synthetic construct
AQPLLRNGEAQ

SEQ ID NO: 1023 - protein, 11 aa, synthetic construct
AQDLQRSDLAQ

SEQ ID NO: 1024 - protein, 11 aa, synthetic construct
AQSSKHSHEAQ

SEQ ID NO: 1025 - protein, 11 aa, synthetic construct
AQVLTPKTNAQ

SEQ ID NO: 1026 - protein, 11 aa, synthetic construct
AQTMIPDTRAQ

SEQ ID NO: 1027 - protein, 11 aa, synthetic construct
AQSYSIPSGAQ

SEQ ID NO: 1028 - protein, 11 aa, synthetic construct
AQHRDMLGIAQ

SEQ ID NO: 1029 - protein, 11 aa, synthetic construct
AQGGGLASTAQ

SEQ ID NO: 1030 - protein, 11 aa, synthetic construct
AQYIVAFEDAQ

SEQ ID NO: 1031 - protein, 11 aa, synthetic construct
AQQLEYVAGAQ

SEQ ID NO: 1032 - protein, 11 aa, synthetic construct
AQMSGSQLIAQ

SEQ ID NO: 1033 - protein, 11 aa, synthetic construct
AQRLQDLRLAQ

SEQ ID NO: 1034 - protein, 11 aa, synthetic construct
AQQLHVPLIAQ

SEQ ID NO: 1035 - protein, 11 aa, synthetic construct
AQSALHRDLAQ

SEQ ID NO: 1036 - protein, 11 aa, synthetic construct
AQKHVANYEAQ

SEQ ID NO: 1037 - protein, 11 aa, synthetic construct
AQLSIQARSAQ

SEQ ID NO: 1038 - protein, 11 aa, synthetic construct
AQVARLVDIAQ

SEQ ID NO: 1039 - protein, 11 aa, synthetic construct
AQLASGVHFAQ

SEQ ID NO: 1040 - protein, 11 aa, synthetic construct
AQMFTETARAQ

SEQ ID NO: 1041 - protein, 11 aa, synthetic construct
AQSSVPSLQAQ

SEQ ID NO: 1042 - protein, 11 aa, synthetic construct
AQNWSGSHTAQ

SEQ ID NO: 1043 - protein, 11 aa, synthetic construct
AQHPTTRLMAQ

SEQ ID NO: 1044 - protein, 11 aa, synthetic construct
AQQSKDRIHAQ

SEQ ID NO: 1045 - protein, 11 aa, synthetic construct
AQTSAVVLGAQ

SEQ ID NO: 1046 - protein, 11 aa, synthetic construct
AQHSGFMVEAQ

SEQ ID NO: 1047 - protein, 11 aa, synthetic construct
AQVSSTVPSAQ

SEQ ID NO: 1048 - protein, 11 aa, synthetic construct
AQSSAKSPTAQ

SEQ ID NO: 1049 - protein, 11 aa, synthetic construct
AQSSTSTVEAQ

SEQ ID NO: 1050 - protein, 11 aa, synthetic construct
AQALDRVHMAQ

SEQ ID NO: 1051 - protein, 11 aa, synthetic construct
AQYAAIDAHAQ

SEQ ID NO: 1052 - protein, 11 aa, synthetic construct
AQDLGSRREAQ

SEQ ID NO: 1053 - protein, 11 aa, synthetic construct
AQSTAHHNLAQ

SEQ ID NO: 1054 - protein, 11 aa, synthetic construct
AQPSPTYSVAQ

SEQ ID NO: 1055 - protein, 11 aa, synthetic construct
AQLISIRSSAQ

SEQ ID NO: 1056 - protein, 11 aa, synthetic construct
AQSYLVAKEAQ

SEQ ID NO: 1057 - protein, 11 aa, synthetic construct
AQKSTTDTSAQ

SEQ ID NO: 1058 - protein, 11 aa, synthetic construct
AQAKAVTLLAQ

SEQ ID NO: 1059 - protein, 11 aa, synthetic construct
AQYTQNGPKAQ

SEQ ID NO: 1060 - protein, 11 aa, synthetic construct
AQTPLTYKPAQ

SEQ ID NO: 1061 - protein, 11 aa, synthetic construct
AQYDVLFKHAQ

SEQ ID NO: 1062 - protein, 11 aa, synthetic construct
AQVDKNASPAQ

SEQ ID NO: 1063 - protein, 11 aa, synthetic construct
AQVRPEAPSAQ

SEQ ID NO: 1064 - protein, 11 aa, synthetic construct
AQETDRYSVAQ

SEQ ID NO: 1065 - protein, 11 aa, synthetic construct
AQSSTSVQWAQ

SEQ ID NO: 1066 - protein, 11 aa, synthetic construct
AQPNSHSQPAQ

SEQ ID NO: 1067 - protein, 11 aa, synthetic construct
AQNPSNVLRAQ

SEQ ID NO: 1068 - protein, 11 aa, synthetic construct
AQHKAESTFAQ

SEQ ID NO: 1069 - protein, 11 aa, synthetic construct
AQLDGTHRSAQ

SEQ ID NO: 1070 - protein, 11 aa, synthetic construct
AQVPRIGSHAQ

SEQ ID NO: 1071 - protein, 11 aa, synthetic construct
AQLDMGRMPAQ

SEQ ID NO: 1072 - protein, 11 aa, synthetic construct
AQSLRDMVFAQ

SEQ ID NO: 1073 - protein, 11 aa, synthetic construct
AQSGQHSGGAQ

SEQ ID NO: 1074 - protein, 11 aa, synthetic construct
AQTGSSHHYAQ

SEQ ID NO: 1075 - protein, 11 aa, synthetic construct
AQAMPTTDLAQ

SEQ ID NO: 1076 - protein, 11 aa, synthetic construct
AQLREDGRVAQ

SEQ ID NO: 1077 - protein, 11 aa, synthetic construct
AQGVVSGHPAQ

SEQ ID NO: 1078 - protein, 11 aa, synthetic construct
AQHGNLLALAQ

SEQ ID NO: 1079 - protein, 11 aa, synthetic construct
AQSWSSDLDAQ

SEQ ID NO: 1080 - protein, 11 aa, synthetic construct
AQSSQSGLSAQ

SEQ ID NO: 1081 - protein, 11 aa, synthetic construct
AQKYVPTVTAQ

SEQ ID NO: 1082 - protein, 11 aa, synthetic construct
AQNALVPFPAQ

SEQ ID NO: 1083 - protein, 11 aa, synthetic construct
AQTYVSSYAAQ

SEQ ID NO: 1084 - protein, 11 aa, synthetic construct
AQYGSSITAAQ

SEQ ID NO: 1085 - protein, 11 aa, synthetic construct
AQVLADSMSAQ

SEQ ID NO: 1086 - protein, 11 aa, synthetic construct
AQYRHSSHDAQ

SEQ ID NO: 1087 - protein, 11 aa, synthetic construct
AQNILALNAAQ

SEQ ID NO: 1088 - protein, 11 aa, synthetic construct
AQKIGISVGAQ

SEQ ID NO: 1089 - protein, 11 aa, synthetic construct
AQSKSFTEEAQ

SEQ ID NO: 1090 - protein, 11 aa, synthetic construct
AQILHSLNGAQ

SEQ ID NO: 1091 - protein, 11 aa, synthetic construct
AQHSGNYVSAQ

SEQ ID NO: 1092 - protein, 11 aa, synthetic construct
AQHVQPGTFAQ

SEQ ID NO: 1093 - protein, 11 aa, synthetic construct
AQMTTVPDVAQ

SEQ ID NO: 1094 - protein, 11 aa, synthetic construct
AQLPLNMLHAQ

SEQ ID NO: 1095 - protein, 11 aa, synthetic construct
AQTNQISTQAQ

SEQ ID NO: 1096 - protein, 11 aa, synthetic construct
AQGSALAHFAQ

SEQ ID NO: 1097 - protein, 11 aa, synthetic construct
AQSVTPLSAAQ

SEQ ID NO: 1098 - protein, 11 aa, synthetic construct
AQLGSTTSDAQ

SEQ ID NO: 1099 - protein, 11 aa, synthetic construct
AQNLSRSVLAQ

SEQ ID NO: 1100 - protein, 11 aa, synthetic construct
AQGPLLGQVAQ

SEQ ID NO: 1101 - protein, 11 aa, synthetic construct
AQKQNMPEDAQ

SEQ ID NO: 1102 - protein, 11 aa, synthetic construct
AQYQSGHAGAQ

SEQ ID NO: 1103 - protein, 11 aa, synthetic construct
AQWTPNGQGAQ

SEQ ID NO: 1104 - protein, 11 aa, synthetic construct
AQSATSSTHAQ

SEQ ID NO: 1105 - protein, 11 aa, synthetic construct
AQSLMPVHTAQ

SEQ ID NO: 1106 - protein, 11 aa, synthetic construct
AQDTSKWESAQ

SEQ ID NO: 1107 - protein, 11 aa, synthetic construct
AQMPGSRPMAQ

SEQ ID NO: 1108 - protein, 11 aa, synthetic construct
AQYIGANSAAQ

SEQ ID NO: 1109 - protein, 11 aa, synthetic construct
AQHTNAHAAAQ

SEQ ID NO: 1110 - protein, 11 aa, synthetic construct
AQMSGGNLTAQ

SEQ ID NO: 1111 - protein, 11 aa, synthetic construct
AQTSYLNVQAQ

SEQ ID NO: 1112 - protein, 11 aa, synthetic construct
AQSMTVSAVAQ

SEQ ID NO: 1113 - protein, 11 aa, synthetic construct
AQTYSVPKEAQ

SEQ ID NO: 1114 - protein, 11 aa, synthetic construct
AQPTDRSLIAQ

SEQ ID NO: 1115 - protein, 11 aa, synthetic construct
AQSKRDMLPAQ

SEQ ID NO: 1116 - protein, 11 aa, synthetic construct
AQITNHLEKAQ

SEQ ID NO: 1117 - protein, 11 aa, synthetic construct
AQDSRPPEPAQ

SEQ ID NO: 1118 - protein, 11 aa, synthetic construct
AQYISSGLHAQ

SEQ ID NO: 1119 - protein, 11 aa, synthetic construct
AQHSASESRAQ

SEQ ID NO: 1120 - protein, 11 aa, synthetic construct
AQSEIHTHVAQ

SEQ ID NO: 1121 - protein, 11 aa, synthetic construct
AQSKMSLVEAQ

SEQ ID NO: 1122 - protein, 11 aa, synthetic construct
AQSPAGTMRAQ

SEQ ID NO: 1123 - protein, 11 aa, synthetic construct
AQLINHPHGAQ

SEQ ID NO: 1124 - protein, 11 aa, synthetic construct
AQALLSNIPAQ

SEQ ID NO: 1125 - protein, 11 aa, synthetic construct
AQNNSTELPAQ

SEQ ID NO: 1126 - protein, 11 aa, synthetic construct
AQLRGAMALAQ

SEQ ID NO: 1127 - protein, 11 aa, synthetic construct
AQSGNSHYMAQ

SEQ ID NO: 1128 - protein, 11 aa, synthetic construct
AQHATHAHPAQ

SEQ ID NO: 1129 - protein, 11 aa, synthetic construct
AQPVGNLTLAQ

SEQ ID NO: 1130 - protein, 11 aa, synthetic construct
AQSSREPEVAQ

SEQ ID NO: 1131 - protein, 11 aa, synthetic construct
AQNLGMAQFAQ

SEQ ID NO: 1132 - protein, 11 aa, synthetic construct
AQLQGIGSAAQ

SEQ ID NO: 1133 - protein, 11 aa, synthetic construct
AQSSRGGLNAQ

SEQ ID NO: 1134 - protein, 11 aa, synthetic construct
AQNLQVTHKAQ

SEQ ID NO: 1135 - protein, 11 aa, synthetic construct
AQSLHMHTVAQ

SEQ ID NO: 1136 - protein, 11 aa, synthetic construct
AQSMISSLSAQ

SEQ ID NO: 1137 - protein, 11 aa, synthetic construct
AQLGTRVNAAQ

SEQ ID NO: 1138 - protein, 11 aa, synthetic construct
AQFGHELSHAQ

SEQ ID NO: 1139 - protein, 11 aa, synthetic construct
AQDSRPTTVAQ

SEQ ID NO: 1140 - protein, 11 aa, synthetic construct
AQWGHLADHAQ

SEQ ID NO: 1141 - protein, 11 aa, synthetic construct
AQSPATTHTAQ

SEQ ID NO: 1142 - protein, 11 aa, synthetic construct
AQSPLLRVEAQ

SEQ ID NO: 1143 - protein, 11 aa, synthetic construct
AQLVESFVRAQ

SEQ ID NO: 1144 - protein, 11 aa, synthetic construct
AQTQHGINFAQ

SEQ ID NO: 1145 - protein, 11 aa, synthetic construct
AQFYDGLTTAQ

SEQ ID NO: 1146 - protein, 11 aa, synthetic construct
AQAKGSTFNAQ

SEQ ID NO: 1147 - protein, 11 aa, synthetic construct
AQMPPAVVSAQ

SEQ ID NO: 1148 - protein, 11 aa, synthetic construct
AQAWPSHDKAQ

SEQ ID NO: 1149 - protein, 11 aa, synthetic construct
AQYMHSLSYAQ

SEQ ID NO: 1150 - protein, 11 aa, synthetic construct
AQAFGDSLLAQ

SEQ ID NO: 1151 - protein, 11 aa, synthetic construct
AQTMTNEGKAQ

SEQ ID NO: 1152 - protein, 11 aa, synthetic construct
AQDRVVVYGAQ

SEQ ID NO: 1153 - protein, 11 aa, synthetic construct
AQLDSVKMLAQ

SEQ ID NO: 1154 - protein, 11 aa, synthetic construct
AQSLSSVSHAQ

SEQ ID NO: 1155 - protein, 11 aa, synthetic construct
AQYRANDSYAQ

SEQ ID NO: 1156 - protein, 11 aa, synthetic construct
AQHHGVQTLAQ

SEQ ID NO: 1157 - protein, 11 aa, synthetic construct
AQYIPGYQSAQ

SEQ ID NO: 1158 - protein, 11 aa, synthetic construct
AQDFSSSRFAQ

SEQ ID NO: 1159 - protein, 11 aa, synthetic construct
AQPTTSALHAQ

SEQ ID NO: 1160 - protein, 11 aa, synthetic construct
AQGGQSPAAAQ

SEQ ID NO: 1161 - protein, 11 aa, synthetic construct
AQNPVQAILAQ

SEQ ID NO: 1162 - protein, 11 aa, synthetic construct
AQTATLYVEAQ

SEQ ID NO: 1163 - protein, 11 aa, synthetic construct
AQVVTKEEKAQ

SEQ ID NO: 1164 - protein, 11 aa, synthetic construct
AQITHSSNTAQ

SEQ ID NO: 1165 - protein, 11 aa, synthetic construct
AQVHLKSGDAQ

SEQ ID NO: 1166 - protein, 11 aa, synthetic construct
AQSNSATNGAQ

SEQ ID NO: 1167 - protein, 11 aa, synthetic construct
AQQTLAGNYAQ

SEQ ID NO: 1168 - protein, 11 aa, synthetic construct
AQRAQNQSGAQ

SEQ ID NO: 1169 - protein, 11 aa, synthetic construct
AQTDAVPRIAQ

SEQ ID NO: 1170 - protein, 11 aa, synthetic construct
AQKLDSVSHAQ

SEQ ID NO: 1171 - protein, 11 aa, synthetic construct
AQDKMRTDTAQ

SEQ ID NO: 1172 - protein, 11 aa, synthetic construct
AQLRSTVEDAQ

SEQ ID NO: 1173 - protein, 11 aa, synthetic construct
AQGISVLQYAQ

SEQ ID NO: 1174 - protein, 11 aa, synthetic construct
AQAPTGTAFAQ

SEQ ID NO: 1175 - protein, 11 aa, synthetic construct
AQTIRTPIHAQ

SEQ ID NO: 1176 - protein, 11 aa, synthetic construct
AQPFAKQLGAQ

SEQ ID NO: 1177 - protein, 11 aa, synthetic construct
AQPASNSTWAQ

SEQ ID NO: 1178 - protein, 11 aa, synthetic construct
AQNMAPSMAAQ

SEQ ID NO: 1179 - protein, 11 aa, synthetic construct
AQAVTVQIGAQ

SEQ ID NO: 1180 - protein, 11 aa, synthetic construct
AQRMPGSVSAQ

SEQ ID NO: 1181 - protein, 11 aa, synthetic construct
AQNMTKITSAQ

SEQ ID NO: 1182 - protein, 11 aa, synthetic construct
AQGYTHSMHAQ

SEQ ID NO: 1183 - protein, 11 aa, synthetic construct
AQHEYFHNEAQ

SEQ ID NO: 1184 - protein, 11 aa, synthetic construct
AQSHAVYVTAQ

SEQ ID NO: 1185 - protein, 11 aa, synthetic construct
AQTVAATHHAQ

SEQ ID NO: 1186 - protein, 11 aa, synthetic construct
AQLVPSHIPAQ

SEQ ID NO: 1187 - protein, 11 aa, synthetic construct
AQSLQERIDAQ

SEQ ID NO: 1188 - protein, 11 aa, synthetic construct
AQNAGDSRVAQ

SEQ ID NO: 1189 - protein, 11 aa, synthetic construct
AQVFASSPSAQ

SEQ ID NO: 1190 - protein, 11 aa, synthetic construct
AQLYSNLTHAQ

SEQ ID NO: 1191 - protein, 11 aa, synthetic construct
AQLGSEHMVAQ

SEQ ID NO: 1192 - protein, 11 aa, synthetic construct
AQFKVSDLSAQ

SEQ ID NO: 1193 - protein, 11 aa, synthetic construct
AQASHDARFAQ

SEQ ID NO: 1194 - protein, 11 aa, synthetic construct
AQFLKMEAWAQ

SEQ ID NO: 1195 - protein, 11 aa, synthetic construct
AQTAISNRSAQ

SEQ ID NO: 1196 - protein, 11 aa, synthetic construct
AQEDSIDTPAQ

SEQ ID NO: 1197 - protein, 11 aa, synthetic construct
AQNSLGLSSAQ

SEQ ID NO: 1198 - protein, 11 aa, synthetic construct
AQLMVNTSRAQ

SEQ ID NO: 1199 - protein, 11 aa, synthetic construct
AQAKVDFLNAQ

SEQ ID NO: 1200 - protein, 11 aa, synthetic construct
AQTSEPRAVAQ

SEQ ID NO: 1201 - protein, 11 aa, synthetic construct
AQFKATFTGAQ

SEQ ID NO: 1202 - protein, 11 aa, synthetic construct
AQTLTQHPIAQ

SEQ ID NO: 1203 - protein, 11 aa, synthetic construct
AQIATSVQDAQ

SEQ ID NO: 1204 - protein, 11 aa, synthetic construct
AQHLAQNSSAQ

SEQ ID NO: 1205 - protein, 11 aa, synthetic construct
AQWDSVLGRAQ

SEQ ID NO: 1206 - protein, 11 aa, synthetic construct
AQRTPDLDVAQ

SEQ ID NO: 1207 - protein, 11 aa, synthetic construct
AQGLGIILSAQ

SEQ ID NO: 1208 - protein, 11 aa, synthetic construct
AQLSTLGLAAQ

SEQ ID NO: 1209 - protein, 11 aa, synthetic construct
AQGSLGFPSAQ